SEQUENCE LISTING (101 sequences)

SEQ ID NO: 1 - protein, 363 aa, Scheffersomyces stipitis
  source 1..363: note = Formerly Pichia stipitis; referred to as Pichia stipitis in the priority application and the present application
MTANPSLVLNKIDDISFETYDAPEISEPTDVLVQVKKTGICGSDIHFYAHGRIGNFVLTKPMVLGHESAGTVVQVGKGVTSLKVGDNVAIEPGIPCRFCDECKSGHYNLCPHMAFAATPNSKEGEPNPPGTLCKYFKSPEDFLVKLPDHVSLELGALVEPLSVGVHASKLGSVAFGDYVAVFGAGPVGLLAAAVAKTFGAKGVIVVARSDRKLKMAKDIGAATHTFNSKTGGSEELIKAFGGNVPNVVLECTGAEPCIKLGVDAIAPGGRFVQVGNAAGPVSFPITVFAMKELTLFGSFRYGFNDYKTAVGIFDTNYQNGRENAPIDFEQLITHRYKFKDAIEAYDLVRAGKGAVKCLIDGPE

SEQ ID NO: 2 - protein, 550 aa, Hypsizygus marmoreus
MSSQVSGPLFLGLDLSTQQLKAILISEDHTVVHESAVHFDNDLPSYGTTNGAIRGPGVGEVTSPVAMWLEAIDLLMDRMKKADVKLDAISAVSGAGQQHGSVYWSNDAEALLSSLDPAQSLVSQLSPGAFSLPQAPIWQDSSTTQECRDLEQAVGGPQVLADITGSRAYERFTGNQIAKIRRLHPESYNATTRISLVSSFIPSVFLGYIAPIDTSDASGMNLMNVLTCKWDDDLLNACGGEMLRSKLGPEPVIGGTVLGKVHQWWVERWGLNPDCIVAPFTGDNPASVMALSAPGDAILSLGTSTTFLLSIPPADTPPQRFTTSHLLSHPTDLDHTIAMLCYKNGALAREQVRDHNADGHWTRFNELVEQTPPGNDGYIGLYFPLPEIIPPGVVGQHAFHVESLDNTAPLAVKDLPEKAHPRAILESQFLSIRSRIADILPEHAPHLKRLVATGGSSANQTIRQLAADIFDMKVFISTTKEAAGMGGALLAKYSWWKTQHTNGRTFEDMTGGEITGLQCVAEPRGSVTGVYDTLVGVYRACEEQVVIKCS

SEQ ID NO: 3 - protein, 553 aa, Cylindrobasidium torrendii
MIENPTFLGLDLSTQQLKAVLVQEDCSIVHESAVRFEEDLPKYGTTKGAHKGPDGVVTSPVAMWLDALDLLMEKMRKAGVDFASIAAISGAGQQHGSVYWSNAAEECLQALDASKPLTQLAPAAFSLQTSPIWQDSSTTRECREIEEAIGGAQALADISGSRAYERFTGTQIKKLRRVNRAAYDSTSRVSLVSSWIPSIFLGTVAPIEISDASGMNLMDVISCKWDDRLLEICGGPELRSKLGPEPVPGGTSLGTVCAWWVERWGFSPECIVAPFTGDNPASVAALSLPGDALLSLGTSTTYLLSIPPASEAPKRFTTSHLLSHPTTAPDAQIAMLCYKNGALAREQVRDRFADADWSKYNDAVEQTHPGSEKYMGLYFPLPEIIPPGVKGSFVYRTEMGKKPVKVEEKDVPTHLHPRMILESQFLSIRSRIAAILPPNSPPLQRLVVTGGSSANQTIRQLAADLFDMKVYVSATKEAAGMGGALLARYAWWRATEDDMGTFEEMCAGLGSEALGMKCVAEPNHAAALEYAQLVEPYVACEAEVVRECNAHLP

SEQ ID NO: 4 - protein, 549 aa, Pleurotus ostreatus
MAATSSQPLFLGLDLSTQQLKAVVITQDLDIVHESAVNFARDLPQYGTENGAIKGPDEGEVTSPVEMWVEAMDLLLARMKEASVDFAAIVAVSGAGQQHGSVFWSKDAESRLSSLDPSRTLLEQLSPTAFSLMNSPIWQDSSTTKQCLELEESIGGPQALADLTGSRAYERFTGTQIAKIHQTRPEVYAPTSRISLVSSFIPSLFLGSVAPIEVSDASGMNLMNVLTCKWDDRLLEACGGSALRSKIGPEPVSGGSNLGKVHKWWVERWGFQPDCIVAPFTGDNPATVVSLSAPGDALLSLGTSTTFLLSIPPADTPPKRFTTSHLLSHPTNLDAQIGMLCYKNGALAREQIRDRYADNDWGKFNTLLEQAPVGNNGLLGFYFPLPEIIPPGVVGEFFYNVDGATPALVEGLPDNAHPRAILESQFLSILSRVEAILPEHAPHLKRLVLTGGSSANQTIRQLAADLFGMKAYVSATKEAAGLGGAMLAKYAWWKQINGGKGTFEAMTGGEMPGMTCVAEPRDEVTQIYEGLVSVYRLCEEDVVSRSQRK

SEQ ID NO: 5 - protein, 566 aa, Trametes coccinea
MLDDPLFLGLDLSTQGLKAVLIDADGAPVHESSVNFDRDLPSYGTTNGAIRGPGAGEVTSPVRMWLDAYDLIMERMRSAGVDFSRILGISGDGQQHGSVYWSTEAERLLASLDSSRPLAEQLSPAAFSIQRAPIWQDSSTTRECRELEAAIGGAQALADLTGSRAYERFTANQIAKIRRTDPDSYRATARISLVSSFMPSLFLGSIAPIEVSDASGMNLMNVLTCEWDDRLLEAAGGPELRAKIGPEPAPGGTFLGKVHPYWVQRWGFNPECGVAPFTGDNPATVVALSTPGDAILSLGTSTTLLLSIPPAETAPKRFTTSHLLSHPTTPDAHIAMLCYKNGALAREQVRDQYADGHWTRFNELVEARPVGNEGYMGFYFPLPEIIPPNVQGNFFFSTSPSPSPSSPSSQSSITALPEASMPRPAHPRAILESQFLSIRSRITAILPPDAPPLQRLVLSGGSSRNPVIQQAAADVFGMRVFVAGAEGAAEGGAQLARYARWRARNGGVGSFEEMRAMDGEGEGEGGERRRCVAEPKWERERMYAGLVETYRACEDLVVQMCERAVA

SEQ ID NO: 6 - protein, 551 aa, Antrodiella citrinella
MSTDTTPLFLGLDLSTQGLKAVLITEDSQVVHEASVRFDQDLPQYGTVNGAVRGPGEGEVTSPVKMWLEAIDLLMQRIKATGVQTSRILGVSGDGQQHGSVFWSSDAENLLSTMNPEQGLADQLAPAAFTLPNAPIWQDSSTTRECRELEEAVGGPQALADLTGSRAYERFTGTQIRKIKRVNPEAYNATSHIALVSSFLPSLFLGAIAPIEISDASGMNLMNVLTYKWDEKLLDVAGGYELRSKLGPEPVPGGVVLGKVSEWWVKRWGFNPDCIVAPFTGDNPATVVALSSPGDAILSLGTSTTLLLSIPPSDTAPKRFTSSHLLAHPTTYDAHIAMLCYKNGALARDQVRNKYANADWAKFNELVEQTPTGNNGLLGYYFSLPEIIPPNVVGNFYFSTSPSIRAVPESEVSAAAHPRAVLESQFLSIRSRIQAVLPANSPPLQRLVLSGGSSSNPIIRQLAADVFGMRVFVAGQEGAAEGGAQLARFAWWREENGGRGTFEEMRAFDGEEERMKCVAEPKEDNVRVYAGLVDAYRECEERVVELCAQTA

SEQ ID NO: 7 - protein, 628 aa, Ustilago maydis
MEGTSPFFLGLDASTQALKASLLDVDLNVLSELEIRFDRDLPQYKTTGGISAPAADDDQGTVVAPVMLYVESIDMLAERMRSASWPLSRIRAISAAGQQHASVYFSRAAPRIFTTLSAEKTLTEQVEQAFSRKVVPNWQDSSTVDACKAFEKAVGGAEKLAQMTGSKAHTRFTGPQIYKFRTQQPQAYKDTERIGLVSSFITTLLCVGQGEDESSVIKGIDEADACGMNLLDMRPASSITATKGRIEPGWNQTLLALASGESEGSDASLGGAVELERKLGSIYRDAGTPVAKIGSWWTKKYGFSPDCHVFPGTGDNPATFLAFSLAEREAIISLGTSDTVMVATHQFVPDPDFHAFFHPARLPSSGDAFFNMLVYKSGSLAREWIRDQYCNSDWAAFNADVEKFKLQQDGEQRAGFYWLRPEIIPPGASGIYRYIKPSTCGEWQKVDDFSDKGMNASGILETQMLNYRYRSSSIVAGPTTSSGSAVDTSVLPLQAIYAVGGASSNPTITQTMADVFGCDIVKPVQPAQDGKEWTPANYNFCSVGAAYKAAWGWSRLQLKAGEKVPEFDEFVHTTKSSQAKRAGSVREGTGEVKGSVQVICRPTEGRTQVYTNVIGAWKQLEERAQKEQ

SEQ ID NO: 8 - protein, 246 aa, Saccharomyces cerevisiae
MAEFSADLCLFDLDGTIVSTTVAAEKAWTKLCYEYGVDPSELFKHSHGARTQEVLRRFFPKLDDTDNKGVLALEKDIAHSYLDTVSLIPGAENLLLSLDVDTETQKKLPERKWAIVTSGSPYLAFSWFETILKNVGKPKVFITGFDVKNGKPDPEGYSRARDLLRQDLQLTGKQDLKYVVFEDAPVGIKAGKAMGAITVGITSSYDKSVLFDAGADYVVCDLTQVSVVKNNENGIVIQVNNPLTRA

SEQ ID NO: 9 - protein, 505 aa, Bacteroides thetaiotaomicron
MYILAHDLGTSGNKATLFDESGLLIASRTAAYPTDYASGNRAEQNPHHWWKAIVDTTQALLELVSPNDIAGVALSGQMMGCLCVDKDGNPLRPHMLYCDQRSQEEEAKLTEKIDPLHFYEITGHRISASYSVEKLMWVKKHEPEIFAQTAKMLNAKDYINYRLCGTIATDPSDASGTNAYDLNRWQWSEEIIEAAELDLSLFPEVRSSIDVIGEITNEAARETGLLAGTPVICGGGDGSCAGVGVGCVAPGTAYNYLGSSSWVALTVEKPIVDEQRRTMNWAHVVPGMLHPSGTMQAAGSSYNWMINQLCQNEQALAAQSGRSVFELIDEQIIASPIGANKLLFLPYMLGERTPRWNVDAKGAFIGLTLGHKHGDMLRAVMEGITLNLGFIINIFRKHVPIDRMTVIGGCAQNPVWRQMMADIYQAEIRVPNYLEEATSMGAAILAGIGAGVFPDFSVIDRFVRIEQTVQPIPENVKKYEAWMPVFDQAYHALCEMYTEIVKTEL

SEQ ID NO: 10 - protein, 623 aa, Scheffersomyces stipitis
  source 1..623: note = Formerly Pichia stipitis; referred to as Pichia stipitis in the priority application and the present application
MTTTPFDAPDKLFLGFDLSTQQLKIIVTDENLAALKTYNVEFDSINSSVQKGVIAINDEISKGAIISPVYMWLDALDHVFEDMKKDGFPFNKVVGISGSCQQHGSVYWSRTAEKVLSELDAESSLSSQMRSAFTFKHAPNWQDHSTGKELEEFERVIGADALADISGSRAHYRFTGLQIRKLSTRFKPEKYNRTARISLVSSFVASVLLGRITSIEEADACGMNLYDIEKREFNEELLAIAAGVHPELDGVEQDGEIYRAGINELKRKLGPVKPITYESEGDIASYFVTRYGFNPDCKIYSFTGDNLATIISLPLAPNDALISLGTSTTVLIITKNYAPSSQYHLFKHPTMPDHYMGMICYCNGSLAREKVRDEVNEKFNVEDKKSWDKFNEILDKSTDFNNKLGIYFPLGEIVPNAAAQIKRSVLNSKNEIVDVELGDKNWQPEDDVSSIVESQTLSCRLRTGPMLSKSGDSSASSSASPQPEGDGTDLHKVYQDLVKKFGDLYTDGKKQTFESLTARPNRCYYVGGASNNGSIIRKMGSILAPVNGNYKVDIPNACALGGAYKASWSYECEAKKEWIGYDQYINRLFEVSDEMNSFEVKDKWLEYANGVGMLAKMESELKH

SEQ ID NO: 11 - protein, 600 aa, Saccharomyces cerevisiae
MLCSVIQRQTREVSNTMSLDSYYLGFDLSTQQLKCLAINQDLKIVHSETVEFEKDLPHYHTKKGVYIHGDTIECPVAMWLEALDLVLSKYREAKFPLNKVMAVSGSCQQHGSVYWSSQAESLLEQLNKKPEKDLLHYVSSVAFARQTAPNWQDHSTAKQCQEFEECIGGPEKMAQLTGSRAHFRFTGPQILKIAQLEPEAYEKTKTISLVSNFLTSILVGHLVELEEADACGMNLYDIRERKFSDELLHLIDSSSKDKTIRQKLMRAPMKNLIAGTICKYFIEKYGFNTNCKVSPMTGDNLATICSLPLRKNDVLVSLGTSTTVLLVTDKYHPSPNYHLFIHPTLPNHYMGMICYCNGSLARERIRDELNKERENNYEKTNDWTLFNQAVLDDSESSENELGVYFPLGEIVPSVKAINKRVIFNPKTGMIEREVAKFKDKRHDAKNIVESQALSCRVRISPLLSDSNASSQQRLNEDTIVKFDYDESPLRDYLNKRPERTFFVGGASKNDAIVKKFAQVIGATKGNFRLETPNSCALGGCYKAMWSLLYDSNKIAVPFDKFLNDNFPWHVMESISDVDNENWDRYNSKIVPLSELEKTLI

SEQ ID NO: 12 - protein, 272 aa, Streptomyces avermitilis
MARPTVLVASDLDRTLIYSAAALALTMPDARAPRLLCVEVHENKPLSYMTETAAGLLTELGDAAVFVPTTTRTRKQYQRINLPGPVPKYAICTNGGQLLVDGVPDPDWHDVVTGRLADECAPLDEVQAHLAETADPVWLRKQRVAEDLFVYLVVERELLPEAWVKELAVWAENRGWTVSLQGRKLYAVPKPLTKSAAMREVARRTGAGLTLAAGDSLLDADLLLAADRGWRPGHGELADAEWAAPAVAALSERGVLAGERILREFLRGVREG

SEQ ID NO: 13 - protein, 256 aa, unidentified
  source 1..256: note = Rhodobacteraceae bacterium
MYAELFRLDGRTAFVTGGSRGIGLACAEALGEAGATVALSSRSRDEGEAAAAALQGKGIRAIYLPADLSTEKAAQEAVRRAADELGGLDILVNNAGIARHCDSLALPPETWDEVIATNLTGLFWCCRAALERMVAAGRGSIVNVGSISGFISNLPQNQVAYNASKASVHMLTKSLAGEFAQQNIRINAVAPGYIETAMTRGGLDDPEWSKIWLGMTPMRRSGTPSEVAAAVLFLASDAASYITGSVLTIDGGYTIH

SEQ ID NO: 14 - protein, 352 aa, Bemisia argentifolii
MASDNLSAVLYKQNDLRLEQRPIPEPKEDEVLLQMAYVGICGSDVHYYEHGRIADFIVKDPMVIGHEASGTVVKVGKNVKHLKKGDRVAVEPGVPCRRCQFCKEGKYNLCPDLTFCATPPDDGNLARYYVHAADFCHKLPDNVSLEEGALLEPLSVGVHACRRAGVQLGTTVLVIGAGPIGLVSVLAAKAYGAFVVCTARSPRRLEVAKNCGADVTLVVDPAKEEESSIIERIRSAIGDLPNVTIDCSGNEKCITIGINITRTGGTLMLVGMGSQMVTVPLVNACAREIDIKSVFRYCNDYPIALEMVASGRCNVKQLVTHSFKLEQTVDAFEAARKKADNTIKVMISCRQG

SEQ ID NO: 15 - protein, 356 aa, Bos sp.
  source 1..356: note = Bos indicus x Bos taurus
MAAAKPENLSLVVHGPGDLRLENYPIPEPGPNEVLLKMHSVGICGSDVHYWQHGRIGDFVVKKPMVLGHEASGTVVKVGSLVRHLQPGDRVAIEPGAPRETDEFCKIGRYNLSPTIFFCATPPDDGNLCRFYKHNANFCYKLPDNVTFEEGALIEPLSVGIHACRRAGVTLGNKVLVCGAGPIGLVSLLAAKAMGAAQVVVTDLSASRLSKAKEVGADFILQISNESPQEIAKKVEGLLGSKPEVTIECTGVETSIQAGIYATHSGGTLVLVGLGSEMTSVPLVHAATREVDIKGVFRYCNTWPMAISMLASKSVNVKPLVTHRFPLEKALEAFETSRKGLGLKVMIKCDPNDQNP

SEQ ID NO: 16 - protein, 358 aa, Aspergillus tubingensis
MSTQNTSAQNLSFVLEGIHRVKFEDRPIPEIKDPHDVLVNVRFTGICGSDVHYWEHGSIGQFVVKDPMVLGHESSGVVSKVGSAVTSLKVGDCVAMEPGIPCRRCEPCKAGKYNLCVKMAFAATPPYDGTLAKYYVLPEDFCYKLPESITLQEGALMEPLSVAVHIVKQAGINPGQSVVVFGAGPVGLLCCAVAKAYGASKVIAVDIQKGRLEFAKKYAATATFEPAKAAALENAQRLIAENDLGSGADVAIDASGAEPSVHTGIHVLRAGGTYVQGGMGRSEITFPIMAACTKELNVKGSFRYGSGDYKLAVSLVGSGKVNVKELITGVVKFEDAERAFEEVKAGKGIKTLIGGVDS

SEQ ID NO: 17 - protein, 383 aa, Chaetomium thermophilum
  source 1..383: note = Chaetomium thermophilum var. thermophilum DSM 1495
MAAGKSNLSFVLKKPNEVSFEERPVPKLQSPHDVLVAVNYTGICGSDVHYWVHGAIGHFVVKDPMVLGHESAGTVVEVGSAVTSLKPGDRVALEPGYPCRRCNNCLSGKYNLCPDMVFAATPPYDGTLTGFWTAPADFCYKLPDNVSLQEGALIEPLAVAVHIVRQARVSPGQSVVVMGAGPVGLLCAAVARAFGASKVVSVDIVQSKLDFAKKFAATHTYMSQRVSAEENAKNLLKAADLPGGADVVIDASGAEPSIQTSLHVVRMGGTYVQGGMGKADITFPIMALCLKEVTARGSFRYGSGDYKLAIELVGSGKVDVKQLINGVVPFEKAEDAFKKVREGQVIKILIAGPNEKVENGGLDTTVDEKKLKEAKVNGSTGCC

SEQ ID NO: 18 - protein, 344 aa, unidentified
  source 1..344: note = Candidatus Poribacteria bacterium
MKAAVLHKVRDIRIEELPKPQIVNDDDVLVRVKAVGVCGSDIHFYMHGRIGDFVVEKPLILGHESAGIVEEVGKGVKDLKPGDKVALEPGIPCRRCEFCKSGRYNLCPHVRFFAAPPVNGVFAEYVVHPADFCFKLPENASLEEGAMVEPLSVGLHGAILGGVRPGDWVAVLGSGPIGLMALQASLAMGASRVICVDLYDFRLKLAKELGASETINAKETDPVEAIADLTGGRGVDVVFEAAGVPKTSQQAIEIARPGGTIVHIGLGEQEAVPMNIVKMIFKELRLQGIHRYANIYPRAIKLIASGRVKVKPMITARLPLERVEEALRMPVERPDSTIKVIVEI

SEQ ID NO: 19 - protein, 350 aa, Linepithema humile
MAKDNLTAILYGINDLRLENTNIEEPGDDEVLLEMGCVGICGSDVHYLVNGRIGDFIVKKPMIIGHESSGTVAKLGKNVKNLEVGDRVAIEPGVPCRTCTFCKEGRYNLCKDIVFCATPPVHGSLRRYYKHAADFCFKLPDHVSLEEGALLEPLSVGVHACKRGGIGIDSKVLILGAGPIGLVTLLVAKAMGASKVVITDIVESRLKMAKKLGADNTYLVQKDRSEKDAIADIHAIFGDEPNRTIDASGAQASIRLAILATKSGGVVVLVGNTAPEVQIPLINALVREVDIRGIFRYANDYDDALNLVASGKIDVKPLITHNYKIEETKKAFETSRTGAGGAIKVMIHCK

SEQ ID NO: 20 - protein, 246 aa, Saccharomyces cerevisiae
MPQFSVDLCLFDLDGTIVSTTTAAESAWKKLCRQHGVDPVELFKHSHGARSQEMMKKFFPKLDNTDNKGVLALEKDIAHSYLDTVSLIPGAENLLLSLDVDTETQKKLPERKWAIVTSGSPYLAFSWFETILKNVGKPKVFITGFDVKNGKPDPEGYSRARDLLRQDLQLTGKQDLKYVVFEDAPVGIKAGKAMGAITVGITSSYDKSVLFDAGADYVVCDLTQVSVVKNNENGIVIQVNNPLTRD

SEQ ID NO: 21 - protein, 223 aa, unidentified
  source 1..223: note = Acidobacteria bacterium
MRTFLCSAILFDLDGVLVDSTRSVSRQWRQWAGEHGIDPEKVLAIAHGVRTIEVVQGLAPHLDLEAEVRRIERREAEDTDGVAVMPGAADLVRSIPDGRWCVVTSGTRLLATARLRLGGLPMPGVLVTADDVTNGKPDPEPYLKGAELLGVKPAECLVIEDAPAGIRAAHAGGMKVIALASTYPVSELQEADTVVRSLKSVAISAAAGTLEVRVAGEEMEAAR

SEQ ID NO: 22 - protein, 210 aa, Mesorhizobium sp.
MFPGRKFAAFLFDMDGTVLNSIAAAERVWTKWARRHGLDVASFLPTIHGKRAIETIAALKLPGVDPVVEADALLKAEAADLEGIVPIPGAVAFLKSLPPERWAVVTSAPRELALLRMAAAGIPVPAIMVSAEDVTRGKPAPDCFLLAAKRLGVEARECLVFEDAPAGIAAGEASGASVMVISATHVHPVVTQHASIATYDALGVATDDNG

SEQ ID NO: 23 - protein, 191 aa, Escherichia coli
MRCKGFLFDLDGTLVDSLPAVERAWSNWARRHGLSPEEVLAFIHGKQAITSLRHFMAGKSEADIAAEFTRLEHIEATETEGITALPGAIALLNHLNKAGIPWAIVTSGSMPVARARHKIAGLPAPEVFVTAERVKRGKPEPDAYLLGAQLLGLAPQECVVVEDAPAGVLSGLAAGCHVIAVNAPADTPRLN

SEQ ID NO: 24 - protein, 214 aa, Puerhibacterium puerhi
MRFDVEAVLFDIDGTLVDSTPAVERIWRTWAGRRGLDAEEILRVCHGRRTEDTLALFLPPGERGAAVAELAELELTDLDEVTALPAAPALLARLPRRRWAAVTSGARPIMRARLAAAGLPLPEVLVAAEDVSAGKPDPEGYLKAAAALGADPRRCLVVEDAPAGIAAGAAAGARTLAVATSHDAARLTAADAVVADLTSVALAVLPDRLAVTVG

SEQ ID NO: 25 - DNA, 4153 bp, synthetic construct
  source 1..4153: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatggcagcagcgaaaccggagaacctcagcttggtagtccacggtcctggcgacctccgcctcgagaattacccaatccctgagcctggaccgaacgaagttctccttaagatgcactcggtgggcatttgcggttcggacgtgcactattggcagcacgggagaatcggagacttcgtcgtgaaaaagcccatggtcctcggtcatgaggcatcgggaactgtggtcaaggttggctctttggtccgtcatctgcaacccggtgatcgtgtggccattgagccaggtgcgccaagggaaactgatgaattctgcaagatcggccgctacaacttgagcccaaccatctttttctgcgctacgcctccggacgatggcaatttgtgccgattttacaagcataacgctaacttctgttacaagctgccagacaacgtaacattcgaggagggcgctttgatcgaacctctctcagttgggatccacgcctgtcgccgagctggcgtgacgcttggcaacaaagttctcgtgtgcggcgcgggtccaattggtttggtatctctactcgcagccaaggcaatgggcgccgctcaggtcgtggtcaccgatctctcggcgtcccggcttagtaaggctaaggaagtcggcgccgattttattctccaaatctctaacgagagccctcaggaaatcgccaaaaaagttgagggactgctgggttcaaagcccgaagtcacgatcgagtgtaccggagtcgaaacgagcatacaagccggtatttacgccacccactcgggtggtacacttgtccttgtcggactcggctccgagatgacttcggtccccctcgtacatgcagcgacccgtgaggtcgacatcaagggcgtctttagatattgtaacacttggccgatggctatcagtatgctagcctccaagagcgtgaatgttaagcctctggtcacccaccgtttccccctggagaaagctcttgaagcgttcgaaacatcccgaaagggcctcggtctgaaagtcatgatcaagtgcgaccccaatgaccagaacccgtaataacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatggcaaagttgacgagcgccgtgccggtgctaaccgcgagagatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctggg

SEQ ID NO: 26 - DNA, 4159 bp, synthetic construct
  source 1..4159: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtcgacccagaacacctcggcgcaaaacctaagctttgtcctggaaggcatccacagagtgaaattcgaggaccggccaattcctgagattaaagatccacatgacgtcctggttaacgtccgtttcaccggtatctgcggttcggacgtccattactgggagcacggttcaatcggacaatttgttgtgaaagaccccatggtgctcggccacgagtcgtccggtgtggtcagcaaggtcggttccgcggtaaccagtctcaaggtcggagactgcgtggctatggaacctggtatcccctgcaggcgatgtgagccctgcaaggctggtaagtataatctctgtgtcaaaatggcttttgccgccaccccgccttacgacggcacgctggcgaagtattacgttctccctgaggatttctgttacaagttgccagagagcatcacacttcaggaaggggcccttatggaacccctgtcggtcgccgtgcacattgttaagcaagctggaatcaatcctggtcagtcagttgtcgtatttggagccggccctgtaggcttgttgtgctgcgcagtagccaaggcttacggtgcttcaaaagttatcgcagtcgatatccagaagggccgactcgagttcgcgaaaaagtacgccgcgacggcaacattcgagccggcgaaagctgccgctctcgagaacgctcagaggctcatcgccgagaatgacctggggagcggtgcagacgtcgccatcgatgcatcgggagccgaaccctctgtgcacaccggcattcatgtgctgcgcgccggcggcacgtacgtccagggcggtatgggccgttccgagataactttcccgattatggctgcctgcacaaaagagttgaacgtcaagggatctttccgctatggttccggggattacaagcttgcagtctctctcgtgggtagtggcaaggtcaacgttaaggaactcatcacgggcgtggtgaagtttgaagatgcagaacgcgccttcgaggaagtcaaggcgggcaagggcatcaagactcttatcggcggcgtcgacagctaataacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatggcaaagttgacgagcgccgtgccggtgctaaccgcgagagatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctggg

SEQ ID NO: 27 - DNA, 4234 bp, synthetic construct
  source 1..4234: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatggccgcgggtaagagcaacttgtccttcgtgctcaaaaagccaaacgaagtatcgttcgaggaacgtcctgttcccaagcttcagtcgccccacgacgtcctcgttgcagtcaattacactggaatttgcggcagtgacgtccattactgggtccacggtgctataggacactttgtggtcaaagatccgatggtgttgggtcatgagtccgctggtaccgtcgtcgaggtcgggagcgcagtcacctcgctgaagccaggcgaccgggtcgcgttggaacctggttatccttgtcgacgctgcaacaactgcctgtcaggtaagtataatctgtgtcctgatatggtgttcgccgctacgcctccgtacgacggcaccctgaccggattctggacggctccggccgatttctgctacaagctccccgacaatgtttccttgcaggagggtgctctaatcgagcctcttgccgtcgcagtacacatcgtgcgtcaggctagagtgtctccaggacaatccgttgtcgtcatgggcgcgggccccgtgggcctgctctgcgcggctgtcgcaagggcatttggagcatcaaaggtcgtgagcgtggacatcgttcaatctaaactggactttgccaaaaagttcgctgcgacccatacatatatgtctcagcgtgtatcggcagaagagaacgctaaaaacctccttaaggccgcagatctccctggtggcgccgatgtcgttatcgatgcctctggagccgagcccagcatccagacatcccttcacgttgtacgtatgggcggaacatacgtccagggcggtatgggaaaggcggacatcaccttcccaatcatggctctttgcctcaaagaggttactgcccgaggcagctttaggtacggctcaggcgactacaagctcgccattgagttggttggtagtggcaaagtcgatgtcaagcaattaattaacggtgtggtgccgtttgaaaaggccgaggacgccttcaaaaaggtgcgcgaggggcaagtcattaagatcctcatcgcgggcccgaacgaaaaggtcgagaacggcggcctcgacacgacggtagacgagaaaaagctcaaggaagcaaaggttaacggttcgaccggctgttgctaataacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatggcaaagttgacgagcgccgtgccggtgctaaccgcgagagatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctggg

SEQ ID NO: 28 - DNA, 4117 bp, synthetic construct
  source 1..4117: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgaaagccgcagttctccacaaagtgcgggacattaggatcgaggaattgcctaagcctcaaatcgtgaacgacgacgacgtcctcgtccgagtgaaagccgtcggtgtttgcggatccgacatccacttttacatgcatggcaggatcggcgatttcgtcgtggaaaagccgctgatcctcggacacgaatcagccggaatcgtcgaagaggtcggtaagggtgtcaaggacctcaagccaggcgacaaagtcgcgttggagcccggtattccttgccgccgttgcgagttctgtaagagcggccgctacaatttgtgtccacacgtccggttctttgccgcaccccctgttaatggcgtgttcgctgagtatgtcgtccaccctgcagatttttgcttcaagcttccagagaacgcttcccttgaggagggtgctatggttgagcctctctcggtgggacttcatggcgcaatcctgggcggtgtacgaccgggcgattgggtggcagttcttggttctggaccgatcgggctgatggccctccaggcaagcctcgcgatgggcgccagtcgtgtcatctgcgttgatctctacgatttcaggttgaaactcgctaaggaactcggcgcgtctgaaactattaacgccaaggaaacagatcccgtagaggctatcgcggacttgacgggtggccgtggcgtcgatgttgtattcgaggccgcgggtgtccctaagacttcgcaacaggccatcgagatcgctcgccccgggggcaccatcgtccatatcggtctaggcgaacaggaagctgtgccaatgaacatcgtgaagatgatctttaaagagctgcgtctgcagggcattcatcgatacgccaacatctatccgcgagctatcaagttgattgcgtcgggaagagttaaggtaaagcccatgataacggcccgcctacccctggagcgtgtggaagaggcacttagaatgcccgtcgagcgcccagactcgaccattaaggtcattgtggaaatctaataacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatggcaaagttgacgagcgccgtgccggtgctaaccgcgagagatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctggg

SEQ ID NO: 29 - DNA, 4135 bp, synthetic construct
  source 1..4135: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatggcaaaagacaatctcacggcgatcctttatggcatcaacgatctccgcttggagaacactaacatcgaggagccgggggacgacgaagtgcttctcgaaatgggctgtgtgggaatctgtgggtcggacgttcattacctggtgaacggccggatcggcgatttcatcgtcaaaaagcccatgatcatcggccatgagtcatctggtaccgtcgccaaattgggtaaaaacgtcaaaaacctagaggtcggagacagggtcgccattgaacctggtgtcccatgcagaacatgcacgttttgcaaggagggtcgatacaatttgtgcaaggatatcgtgttttgcgcaacgcctcctgttcatggctcccttcgccgctactacaagcacgcagctgacttctgctttaagttgccggatcacgtgtccctggaagagggtgctctcctcgagcccctgagcgttggcgtccacgcgtgcaagcgtggtggcatcggcatcgactctaaggtactgatcctcggagccggaccaatcggcctggtgacccttctcgtcgccaaggctatgggtgcatcaaaagttgtgattactgatatagtggagagccgcctgaagatggctaaaaagctcggtgctgataacacctacctggtacaaaaggatcgatcggaaaaggacgcaatcgccgacatacacgcgattttcggtgatgaacccaaccgtacaatcgacgcttcgggagcgcaggcttccatccgactcgccattctagcgactaagagtggcggagtcgtcgtgcttgttggcaacacagctccagaagttcagattcccttgatcaatgccctcgtcagggaggtcgatatccgtggtattttcagatacgccaatgactatgacgacgcactcaatctggtagcctcgggtaagatcgacgtgaagcctctcatcacccacaactacaagatcgaagagactaaaaaggcatttgaaaccagccgaacgggcgccggcggcgccattaaggtcatgatccactgtaagtaataacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatggcaaagttgacgagcgccgtgccggtgctaaccgcgagagatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctggg

SEQ ID NO: 30 - DNA, 3648 bp, synthetic construct
  source 1..3648: mol_type = other DNA
agatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctgggggcgtgagttcgcactccgagatccagctggaaattgtgttcatttcgtagctgaggaacaggattaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatgccgcaattttcagtagacctctgcctctttgacttggatggcacgatcgtgtcgaccaccaccgccgctgagagcgcgtggaaaaagctgtgcagacagcatggtgtcgacccagtcgagctgttcaagcattcacacggcgcccgcagccaggagatgatgaaaaagttctttccaaagctcgacaataccgataacaagggtgtgcttgccctcgaaaaagatatcgcacactcttacttggatacagtcagtctcattcccggtgctgagaatcttttgctgtccctagacgtggataccgagactcagaaaaagctccctgagcgaaagtgggccattgtcacaagcggatcgccttacctggcgttttcctggttcgaaacgatcttgaaaaacgtcggcaagcccaaggtattcatcaccggtttcgacgtgaaaaacggtaagccagatcctgagggctattcgcgggctcgtgaccttctgaggcaagacctccagctcacgggcaagcaagatctgaaatacgtcgttttcgaagatgctccggtcggcattaaggccggcaaggcaatgggcgcgatcactgtcgggatcacgagctcttacgacaagtccgttcttttcgacgccggagcagactacgttgtgtgtgacctcacacaagtctcggttgtgaaaaacaacgaaaacggaatcgtcatccaggtgaataaccccctcactcgcgactaataacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 31 - DNA, 3579 bp, synthetic construct
  source 1..3579: mol_type = other DNA
agatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctgggggcgtgagttcgcactccgagatccagctggaaattgtgttcatttcgtagctgaggaacaggattaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatgcgaacctttctctgctccgccatccttttcgatcttgacggcgtcctcgtcgactcaacccgcagcgtcagccggcaatggcgtcagtgggcgggagaacatggtatcgacccagagaaagtattggcaatcgcacacggcgttagaacgattgaagtcgtgcagggtcttgccccacacctcgacctggaagctgaagtcagacgtatcgagaggcgtgaggccgaggatactgatggtgttgctgttatgccgggcgccgcagatctggtgcgatcgattcctgacgggcgttggtgtgttgtaacatcgggtacacgcctcttggccaccgctcgtctacgcctcggcggactgcccatgcctggtgtgttggtgacggccgacgatgtcactaacggaaagcccgacccggagccgtacctcaagggcgctgagctcctgggtgtgaagccagctgagtgcctggtcatcgaagatgctcctgctggcattcgagcggcgcatgcgggtggaatgaaagtaatcgcattggcgtctacgtaccccgtgagtgagctccaagaggccgacaccgtcgtccgctcgcttaagtctgttgccatctccgccgcagctggcaccctcgaagtccgcgtggcgggcgaagagatggaagccgcaaggtaataacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 32 - DNA, 3540 bp, synthetic construct
  source 1..3540: mol_type = other DNA
agatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctgggggcgtgagttcgcactccgagatccagctggaaattgtgttcatttcgtagctgaggaacaggattaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatgttccctggccgtaagttcgccgcgtttctcttcgatatggacggaactgttctcaattcgatcgccgccgcagaacgggtctggaccaaatgggcccgtcgacacggcttggacgtggcttcgtttctccccacgattcacggcaagcgcgcgatcgagacaatcgccgctcttaagctacccggggtcgaccctgtggtcgaagccgacgcactgctcaaagctgaggccgcagatctcgagggcatcgtgcctatcccaggtgccgtggctttcctcaagagtctgcccccggaacgctgggcagttgtgacctctgctccgcgcgagttggcgctgcttaggatggccgccgctggaatccccgtaccagcgattatggtatcggctgaagatgtcactcgtggtaagcctgccccggactgcttccttctcgcagcgaagagactgggcgttgaggctcgagagtgtttggtctttgaagatgcgcccgctggcatcgctgcgggcgaggcctccggtgcaagcgttatggtcatcagcgcaacgcacgtccatccagtggtcacccagcatgcgtcaattgcaacgtacgacgctctgggtgtcgccacagatgacaacggataataacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 33 - DNA, 3483 bp, synthetic construct
  source 1..3483: mol_type = other DNA
agatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctgggggcgtgagttcgcactccgagatccagctggaaattgtgttcatttcgtagctgaggaacaggattaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatgcgttgcaaaggcttcctcttcgatcttgatggaacactcgtggacagtttgcctgctgtcgagcgcgcatggtcgaactgggcccgtaggcacggtctgtctcctgaagaggtcctcgcgtttattcatgggaagcaagctatcacctcgctccgccactttatggccggtaagtccgaggccgacattgccgcagagttcactcgtcttgagcacatcgaagctacagaaacggaaggcatcactgccctcccgggcgcaatcgcgctgctaaaccacttgaataaggctggcatcccttgggctattgttacgagcggttcaatgcccgtggcccgagcaagacataagatcgccggcttgcctgcacccgaagtgttcgtcaccgctgagcgagtcaaacggggtaagccagagccggacgcctaccttctcggtgcgcagctgctcggactggccccacaggaatgtgtcgtggtcgaggatgcgccagcgggcgttctcagcggcctcgcggccggatgccatgttatcgctgtaaacgcacccgctgacaccccgcgcctgaactaataacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 34 - DNA, 3552 bp, synthetic construct
  source 1..3552: mol_type = other DNA
agatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctgggggcgtgagttcgcactccgagatccagctggaaattgtgttcatttcgtagctgaggaacaggattaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatgcgtttcgacgtagaggccgtcttgtttgacatcgacggtacactcgttgactcaacccctgctgtcgagcgaatttggagaacctgggccgggcgtagaggcctcgatgcagaggaaatcctccgggtctgtcacggtcgtcgcaccgaggatactctggccctgttcctcccgcccggtgagaggggcgcagcggttgctgaactcgccgagcttgagttgacggacctcgacgaagtcaccgcgctaccagcagcgcccgcccttctggcccgactcccacgccgccgttgggccgcggtcaccagcggcgcacgtcctatcatgcgagccaggctcgcggccgcaggcctccccttgccggaagtgctcgtcgcagccgaagatgtgtctgccggaaagcccgatcctgagggatacctgaaagctgctgctgcgttgggtgctgacccgcggcgctgcctcgtggttgaagatgctcctgcaggcatcgccgcgggtgccgccgcgggagcacgcacgcttgctgtggcgacatcgcatgacgctgcacgtcttacagccgctgacgctgtcgtcgctgacctgacgtccgtagctttggccgttctgccagatcgacttgcggtcactgtgggctaataacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 35 - DNA, 3968 bp, synthetic construct
  source 1..3968: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactctggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatggccgaattttccgctgatttgtgcctcttcgaccttgacggaactatcgtctcgactaccgttgcggcagagaaggcgtggaccaagctctgctacgaatacggtgtggatccgtcggaactgttcaagcattctcacggtgcccgcacccaggaagttctccgtcgcttctttccgaaactggatgataccgataacaaaggagtgcttgcacttgagaaggatattgctcattcctatctcgacaccgtgtcccttatccctggagccgaaaacctccttctctccctggacgtcgatacagaaactcagaagaaactgccagaacgaaaatgggccattgtcacctccggcagcccctacctggccttctcctggttcgaaactattcttaaaaatgttggcaagcctaaagtctttatcacgggatttgacgtcaagaacggcaaacccgacccagaaggttactctcgtgcgcgagatttgttgcgacaggatctgcagttgaccggaaaacaggatttgaaatatgtcgtcttcgaagacgctcccgttggtatcaaagccggcaaggcgatgggcgccatcacggtcggaatcacttccagctacgacaagtctgtcttgtttgatgcgggtgctgactacgtggtttgcgatttgacacaggtttccgttgtcaaaaataatgagaacggcattgtgattcaagtcaataacccgttgacacgtgcttaacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 36 - DNA, 1949 bp, synthetic construct
  source 1..1949: mol_type = other DNA
gaaggtgtcaagctcgctgtcaaagctggctaccgtcacttggacttggccaaagtttaccagaaccaaaccgagattggccaagctctcaaggaactgtttgatgagggtgttgtcaagcgtgaggaccttttcatcacttccaagctttggaacaaccgccacgctcctgagcacgttgagcctgcgctcgacgagactcttaaggagctgggtctatcctatctggacctgtacttgattcactggcctgttgctttcaagttcactactcccgatgaattgctccctgctgaccctaccaacaaggatcttgcctacgttgacgattcggtaaaattgtccgacacctggaaggcggtcgtcgccctgaagaagacgggtaagaccaagtcggttggtgtttcgaacttcagcactcgtctggtcgacttggttgaggaagcttataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatccatcatgcatcatgcatcatgcatcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccag

SEQ ID NO: 37 - DNA, 1476 bp, synthetic construct
  source 1..1476: mol_type = other DNA
gaaggtgtcaagctcgctgtcaaagctggctaccgtcacttggacttggccaaagtttaccagaaccaaaccgagattggccaagctctcaaggaactgtttgatgagggtgttgtcaagcgtgaggaccttttcatcacttccaagctttggaacaaccgccacgctcctgagcacgttgagcctgcgctcgacgagactcttaaggagctgggtctatcctatctggacctgtacttgattcactggcctgttgctttcaagttcactactcccgatgaattgctccctgctgaccctaccaacaaggatcttgcctacgttgacgattcggtaaaattgtccgacacctggaaggcggtcgtcgccctgaagaagacgggtaagaccaagtcggttggtgtttcgaacttcagcactcgtctggtcgacttggttgaggaagcttataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatccatcatgcatcatgcatcatgcatcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatggcaaagttgacgagcgccgtgccggtgctaaccgcgagagatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctggg

SEQ ID NO: 38 - DNA, 1276 bp, synthetic construct
  source 1..1276: mol_type = other DNA
agatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctgggggcgtgagttcgcactccgagatccagctggaaattgtgttcatttcgtagctgaggaacaggattaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttataaccagatcgaagctcaccccttgttgcaacaagacgagttggttgctcaccataagagcaagaacattgtcatcactgcttacagtcccttgggcaacaatgtcgctggtaaaccacctctgactgagaaccccggtattgtggatgctgctaagcgtttgaaccatactcctgctgctgtgctcattgcttggggtattcaacgcgggtacagcgtcttggtcaagtcagttacaccctcacggatcaagagtaactttgaacagatcactctgtctgatgaggaattccaacgggttaccaacctcatcaaggagtacggtgagagccgtaacaacgttcctttcaattacaagccttcgtggtccattgacgtctttggtacccaggacgaggcta

SEQ ID NO: 39 - DNA, 4447 bp, synthetic construct
  source 1..4447: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtccttgacgggttcaatattcaatactcagtgttcaagcagtggtaggttgctccgtggatggaacgacgggtttaatttcttccgccatgcaccatggtccatgatcggttcagatggacagttggtatgagaaagtggtaacagataaatagaatttcgtacaagttggcaagagcctggccgatggtccatggctcgatggctcgatggctcgatggctcgatggccacacaccatgtcatctactttctgtgcttctatgcggacgagtcggaacagctgtcaaactgcgtttggggtatttgagcggaatttgaaaccttgttgaaactacattgtctcctcgttacttctctggcatctgtttgatcgaacggcgtcatgcaatagacacgcgatgaggcccaacgacggtgacgttgaacgcctggcccacctgctacaacaccgtagaacgtgttgaagttgaacgtcggacgttggacgttgagttgacgcgtgatgctggaaccgtggatgtagcagcgacagacaagccctctaagcaatctagtcgattttcaaaccaactaaacgagccttagttcggacaaccctagtccagtgcgggggcagcgtggggtcagttgccagcgaaggtcaccccagctagctgggttggctagcttgactggctcagaagtatcgatgctggagcttcaggccagtacgaaaatacactgtcgtagtgctccttccggagttctgggcttctgagcccggcggttgtgcatcggtgcacagtgcacggaccggcgggtgggacaacaaactgtctgggcgtgaaatcgtgcgagtgttgatgcaaaccggacgtacccagcacgcagttcccagtactcagacatactcagtacctcagtggttgagcctcagcatcgcctccgtatttctacaaagctgttgagtcgccttttgactttttactaccaccatcccctcttcttctaaaccccttacattccaaaaatgactgccaatccctcgttggtccttaacaaaattgacgatatcagcttcgaaacttatgatgcacccgaaattagcgaaccgactgacgtgttggttcaagtcaagaaaacgggaatttgtggttcggacatccatttttacgcacacggacgcatcggaaacttcgttctgactaaacccatggtcctgggacacgaatccgcaggcacagtcgtccaagtcggcaaaggagtgacaagcttgaaagttggtgataatgttgcaattgagcccggtatcccatgccgcttttgtgacgagtgcaaaagcggtcattacaacctttgcccacacatggccttcgccgcaacacccaacagcaaagagggagaacctaatccacccggaactctttgcaagtatttcaagagccccgaagattttctcgtgaaactgcccgaccatgtgtctcttgaactgggagctctggttgaacccctctcggtcggagtccacgcgtctaaactcggatcggtggcttttggtgattacgttgcagtcttcggagctggccctgttggtctcctcgcggcagcggttgcaaaaacattcggtgcgaagggcgtgattgttgttgcccgttctgaccgaaaattgaaaatggcaaaggatattggagccgctacacacaccttcaactccaaaaccggcggctcggaggagctcattaaggcattcggcggtaatgttcctaatgtggtcctcgaatgcactggtgctgaaccatgcatcaaactgggtgtggatgcgatcgcaccaggaggtcgatttgtccaggtgggaaatgcagcgggccctgtgtcgtttccaattaccgtgtttgctatgaaagaactgaccttgtttggctcttttcgatacggattcaacgactacaaaacggcggttggtattttcgacactaactatcaaaacggccgcgagaacgccccaattgattttgaacaacttattactcaccgctataaattcaaagacgctatcgaagcttatgacttggttcgcgctggcaagggagcagttaagtgcctgatcgacggaccagaataacattcaccctggagtaactgcgcttcatcgtttctttgttacgacaataataccacgctgcatgctttgaattgagtatatattttgatgactggttcaaatagatctttcctccatagccttcgagtactcgtgatctcagcattatcagatttgtgtccaggcccccgaggacgatgcggttttcctggagtcactagttgggaagatttgtactggttgggtgtaggtagagagaaccattgattttggtgtagctgttcgttttataaggccgagattacgtaatgcacatgctgccctagcaaatcgcgtcgcgtcacgcgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 40 - DNA, 4126 bp, synthetic construct
  source 1..4126: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtccttgacgggttcaatattcaatactcagtgttcaagcagtggtaggttgctccgtggatggaacgacgggtttaatttcttccgccatgcaccatggtccatgatcggttcagatggacagttggtatgagaaagtggtaacagataaatagaatttcgtacaagttggcaagagcctggccgatggtccatggctcgatggctcgatggctcgatggctcgatggccacacaccatgtcatctactttctgtgcttctatgcggacgagtcggaacagctgtcaaactgcgtttggggtatttgagcggaatttgaaaccttgttgaaactacattgtctcctcgttacttctctggcatctgtttgatcgaacggcgtcatgcaatagacacgcgatgaggcccaacgacggtgacgttgaacgcctggcccacctgctacaacaccgtagaacgtgttgaagttgaacgtcggacgttggacgttgagttgacgcgtgatgctggaaccgtggatgtagcagcgacagacaagccctctaagcaatctagtcgattttcaaaccaactaaacgagccttagttcggacaaccctagtccagtgcgggggcagcgtggggtcagttgccagcgaaggtcaccccagctagctgggttggctagcttgactggctcagaagtatcgatgctggagcttcaggccagtacgaaaatacactgtcgtagtgctccttccggagttctgggcttctgagcccggcggttgtgcatcggtgcacagtgcacggaccggcgggtgggacaacaaactgtctgggcgtgaaatcgtgcgagtgttgatgcaaaccggacgtacccagcacgcagttcccagtactcagacatactcagtacctcagtggttgagcctcagcatcgcctccgtatttctacaaagctgttgagtcgccttttgactttttactaccaccatcccctcttcttctaaaccccttacattccaaaaatgtatgccgaacttttccgcttggacggacgtacagcttttgttactggcggttcccgtggcattggactcgcctgcgctgaagcccttggtgaagcgggtgctacagtcgcattgagctctcgctcgcgcgacgagggtgaagcagcagccgcggctctccagggaaagggcattcgtgccatttatttgcccgcggatcttagcaccgaaaaagcggctcaagaggcagtgcgacgagcagcagatgagcttggtggccttgatattctggtcaataacgctggcatcgctcgccactgtgattcccttgcgctcccccccgaaacctgggatgaggttattgctactaacctcacaggtttgttctggtgttgccgcgctgcgctggaacgcatggtcgctgcaggccgtggtagcattgtcaacgtgggtagcatttctggttttatctcgaacctgccacaaaatcaagttgcttacaatgcctcgaaggccagtgtccacatgctgacgaaatctctcgcaggagaattcgctcaacagaatatccgcatcaatgcggtggcacccggctatatcgagacagcgatgacccgcggaggactggacgaccccgaatggagcaagatttggcttggtatgactccaatgcgccgaagcggaacaccgtcggaggtggctgcagctgttcttttcctcgcttctgatgcagcatcgtatattacgggctctgtccttacgatcgacggaggatacacgattcactaacattcaccctggagtaactgcgcttcatcgtttctttgttacgacaataataccacgctgcatgctttgaattgagtatatattttgatgactggttcaaatagatctttcctccatagccttcgagtactcgtgatctcagcattatcagatttgtgtccaggcccccgaggacgatgcggttttcctggagtcactagttgggaagatttgtactggttgggtgtaggtagagagaaccattgattttggtgtagctgttcgttttataaggccgagattacgtaatgcacatgctgccctagcaaatcgcgtcgcgtcacgcgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 41 - DNA, 4880 bp, synthetic construct
  source 1..4880: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactctggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatgtcgtcccaggtttctggacctctgtttctcggtttggacctgagcacacaacagctgaaggcgatccttatctcggaggatcatacagtcgtccatgaaagcgcagttcacttcgataacgatttgccctcctatggcaccaccaacggtgcgatccgaggtccaggagttggtgaagttacttctccagtggcgatgtggttggaagcaatcgacctgctcatggatcgcatgaaaaaagccgatgtcaagctcgacgcaatttccgcagtttctggagccggtcaacagcacggttctgtctactggtccaacgacgcagaagcgctcttgtcctctcttgatcctgctcaaagcttggtcagccagctgtcccccggtgctttttcgttgccgcaagcgccaatttggcaagattcctccactacccaagaatgccgcgacctggagcaggctgtcggtggaccgcaagtcctcgccgacatcaccggatcgcgcgcctatgaacgatttactggcaatcagatcgctaaaattcgtcgcttgcacccggaatcctataatgccaccacccgcatttcccttgtgtcttcgttcattccctcggtgttcctcggttacattgccccgatcgatacgtccgatgcctctggtatgaatcttatgaatgttcttacatgcaagtgggacgacgacctgctcaacgcctgcggtggtgagatgttgcgaagcaaactcggaccagaacctgtgatcggcggaactgttctgggaaaagtgcaccaatggtgggtggagcgttggggcttgaatcctgactgtatcgttgcacccttcacgggagacaatccggcatctgttatggccctttccgcaccaggagacgcaattctttctctgggaacatctacaacattcctgcttagcatccctcctgcagacactccaccacaacgttttacgactagccatctgctctcccaccctactgacttggaccatacaatcgcaatgttgtgttataagaacggagctctcgcgcgtgagcaagtgcgtgatcacaacgcggacggccactggacgcgattcaatgagctggtcgagcaaacgcctccgggaaacgacggatatatcggcctctacttccctcttcccgaaatcattccgccaggcgtggtcggccagcatgcctttcatgtcgagtctcttgataacactgcaccactcgccgtcaaagatttgcctgagaaagcacacccgcgcgccatcttggagtctcaatttttgtccattcgatcccgcatcgctgatattctgcctgaacacgcgccgcatctcaagcgcttggttgcgacgggtggctcttctgcaaatcagacgatccgccaactggctgcagacatttttgatatgaaagtcttcatttcgacgactaaggaggctgctggtatgggtggtgctctgctggccaaatattcctggtggaaaacccagcacaccaatggtcgtaccttcgaggacatgacaggtggtgaaattactggactccagtgtgttgcggagccccgtggttctgtcaccggcgtctacgacactctggtcggtgtttatcgtgcctgcgaggagcaagttgtgatcaaatgttcgtaacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 42 - DNA, 4889 bp, synthetic construct
  source 1..4889: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactctggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatgatcgaaaatccgacgttcttgggcttggatctgtcgacccaacaactgaaggcggtccttgtgcaagaagattgctcgattgtccacgagtcggctgtgcgctttgaagaagacttgccaaaatatggcaccaccaaaggagcgcacaaaggtcctgacggcgtggtcacctctcctgtcgcaatgtggcttgacgcccttgatcttttgatggaaaaaatgcgcaaagcaggagtggacttcgcgtcgatcgcagcgatctcgggagcaggacaacagcatggctcggtttattggtcgaacgcggccgaggagtgtctgcaagcccttgatgcttccaagccacttacacaactcgcaccagctgccttttctctgcaaacctctcccatctggcaggactcgtccacaacacgcgaatgccgcgagatcgaggaagctattggaggtgctcaggctctcgccgatatttcgggttctcgcgcttacgagcgtttcacgggcacccagatcaaaaagctgcgtcgtgtgaaccgagctgcctatgactcgacatctcgtgtttcgctcgttagcagctggatcccatcgatttttcttggcaccgttgcaccgatcgaaatctccgatgctagcggtatgaatcttatggacgtgatttcttgcaagtgggacgatcgtctcttggagatttgtggtggcccagaattgcgatccaagctcggtcctgaaccagtgcccggcggtacttctttgggtaccgtttgcgcgtggtgggtcgaacgttggggtttttctccagagtgcattgttgctcctttcacgggcgacaatcccgcgagcgtggcagcgctgtccctccctggtgatgcgttgctgtccctgggaacctctacaacttatttgttgtccatcccgccggcgtctgaggcgcctaaacgttttacgactagccatctgctttcccatcccactactgcgccagacgcacagatcgcgatgctgtgttataagaatggagccctggcacgagaacaagttcgcgaccgatttgcggacgccgactggtcgaaatataacgatgcagttgagcagactcatccaggctctgaaaaatacatgggtctgtattttcccctgccagaaattatcccgcccggcgttaaaggcagctttgtctaccgcacggaaatgggtaagaagcccgtcaaggtcgaggagaaggatgttcccacgcaccttcatccacgaatgattctcgaatcccaatttctttccatccgctcccgaattgctgccatcctgccgccgaattctcctccgttgcaacgcctggttgtgactggaggttcctctgcgaaccagacgatccgccaacttgcggctgacttgtttgatatgaaggtctatgtctcggcaaccaaggaagcggcgggtatgggcggtgccctgctggcgcgttatgcgtggtggcgtgccaccgaggacgatatgggcacatttgaggaaatgtgcgctggccttggaagcgaagccttgggaatgaagtgcgtcgctgaacccaatcacgctgctgcactcgagtatgcccagcttgttgaaccctatgttgcgtgcgaggcagaagttgtgcgtgagtgtaacgcacatttgccgtaacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 43 - DNA, 4877 bp, synthetic construct
  source 1..4877: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactctggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatggcagctacgtcgtcccaacctttgtttcttggcttggacctgtcgactcaacaactcaaagccgttgtgatcacccaggatctggacatcgtccacgagtctgctgttaacttcgctcgagatttgccacaatacggtaccgagaatggagcaatcaagggtccagatgaaggcgaagtcacttcgccagttgaaatgtgggtggaagccatggatcttctgttggctcgaatgaaagaagcttccgtggactttgcagccattgttgcagtctcgggcgccggacaacaacacggcagcgttttctggtctaaggacgcggaatcccgtttgtcgagcctggatcctagccgaaccttgttggaacaactgtctccgaccgcattctcgctgatgaactccccgatttggcaagatagctctacgacgaagcaatgccttgagctcgaagagagcattggtggcccccaagccttggcagatctcacgggtagccgcgcatatgaacgcttcacaggaactcagattgcaaaaattcatcaaacccgtccggaagtctatgcccctacatcgcgcatcagccttgtgtcttcttttattccttctctgttcctgggatccgtcgctccaatcgaagtgtctgatgcatctggtatgaacctgatgaacgttttgacctgtaagtgggacgatcgattgctcgaagcctgtggtggctccgcacttcgatccaagatcggacccgaacccgttagcggaggatcgaatcttggaaaggtgcacaagtggtgggtcgaacgctggggctttcaacctgactgcattgttgcgccatttactggcgacaatcccgcaaccgttgtctcgttgagcgcccctggagatgctcttttgagccttggaacatctacgaccttcctcttgtcgatcccgccggccgacaccccgccgaaacgcttcactacctcgcacctcttgtcgcacccgaccaacttggatgcgcaaattggaatgttgtgctacaaaaacggagctttggcgcgcgaacaaattcgtgatcgctatgcggacaacgactggggcaaattcaatacacttctcgagcaagctcccgtcggaaataatggactcctcggcttctatttcccgttgcctgaaatcatccctccaggtgtcgtcggtgagttcttttacaacgtggatggagctacgccggcacttgttgagggattgcccgacaatgctcacccccgtgctattctggaatctcaattcttgtcgatcttgagccgcgttgaagcaattctgccggagcacgcacctcacttgaaacgcctcgttctcacgggcggtagctctgctaaccaaactattcgtcaactggccgccgatctgtttggcatgaaggcctacgtttcggcaacaaaggaagccgcaggattgggcggcgcaatgttggccaaatacgcatggtggaaacagatcaacggcggcaagggcactttcgaagctatgacgggcggcgaaatgcccggtatgacgtgtgtggccgagccgcgtgatgaagtcactcaaatttacgaaggtcttgtttccgtttaccgcttgtgcgaagaggatgttgtttctcgtagccaacgtaagtaacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 44 - DNA, 4928 bp, synthetic construct
  source 1..4928: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactctggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatgctggatgatcccctctttcttggacttgacctgtccacccaaggtcttaaagcagtcctgattgacgcagacggtgccccggtgcacgaaagctctgtgaactttgatcgagatctcccctcgtacggcactacgaatggagctatccgtggcccgggcgctggtgaggtcaccagccccgtccgaatgtggctcgacgcttatgacctgattatggaacgaatgcgttccgcaggagtcgacttctcgcgaatccttggaatctccggcgatggtcagcagcacggctctgtctattggagcaccgaggcggaacgcctcctcgcgtcgcttgatagcagccgaccattggctgaacagctttcgcccgcggcgttctctattcaacgcgctcctatttggcaagactcttcgaccacgcgagaatgccgtgaattggaggcagccatcggcggtgcgcaggctctcgcggacctcacaggcagccgcgcgtatgagcgattcacagcgaaccaaatcgccaaaattcgacgaacggatccagacagctatcgagccacagctcgtatttcgctggtttcctcgttcatgccttccctttttctcggcagcatcgcacccattgaagtgagcgacgcctcgggaatgaaccttatgaatgtgcttacatgcgaatgggatgatcgacttctcgaagccgcgggaggacccgagcttcgtgcgaagattggacctgaacctgcgccaggcggcaccttcttgggcaaggtgcatccgtactgggtgcaacgctggggatttaaccccgaatgtggagttgcaccattcaccggtgataatcccgcgacagttgtcgccctctcgacaccgggtgacgcaatccttagcctgggtacatccaccacccttctcctgagcatcccacccgcggagactgccccaaagcgatttactactagccatcttcttagccatcctacgactcccgatgcgcatatcgctatgctctgctataagaacggcgccctcgcgcgtgaacaggttcgagatcaatatgcagacggccactggacccgtttcaatgagttggtggaggctcgtccggtcggcaacgagggatatatgggtttctattttccactcccggagatcattccccccaatgtccagggtaacttcttcttctctacctccccgagcccatccccaagcagcccttcttcccagtcctctattaccgctttgcctgaggcatccatgccccgacctgcacatccccgagcaattctggagtctcagtttctctccattcgttcgcgtatcacggcaattcttccgcccgatgctccaccgcttcagcgactcgtcctctcgggtggcagcagccgcaatcctgtcattcaacaggctgcagcagatgttttcggtatgcgagtcttcgttgccggcgcggaaggagcagccgagggaggtgcacaattggcccgctacgcccgttggcgagcacgcaatggcggagttggctccttcgaagagatgcgtgctatggacggcgaaggtgaaggtgagggtggagagcgacgacgatgcgtcgccgagccgaagtgggaacgcgaacgcatgtacgcaggattggttgaaacgtaccgagcgtgcgaggatttggtggtccaaatgtgcgagcgcgctgtcgcctaacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 45 - DNA, 4883 bp, synthetic construct
  source 1..4883: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactctggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatgtccacggacaccacgccattgtttctcggacttgacctgtctacacaaggactcaaagccgttctcatcacggaagactcgcaggtggtgcatgaagcgagcgttcgcttcgaccaggatcttccccaatacggcacagtgaatggtgcagttcgcggccctggagagggcgaagtgacgtctccggtcaaaatgtggcttgaagccatcgatctgttgatgcagcgtatcaaggccaccggagtccaaacttcccgtattctgggagtctcgggcgatggacaacaacacggctcggttttctggtcttcggacgctgagaacttgctctcgactatgaacccggaacaaggcttggcagaccagctcgcgcctgctgctttcacccttccgaacgcccctatctggcaggattctagcacgacgcgtgaatgccgcgagcttgaggaggcggttggtggcccgcaagctttggcagatctcaccggcagccgagcctatgaacgctttacgggcacccaaatccgaaagattaaacgcgttaatcccgaagcatataatgcgacttcgcatatcgcgctcgtctcgagcttcttgccatccttgttcctgggcgcaattgcgccaattgaaatttcggatgcgagcggaatgaatctcatgaatgttctgacttataagtgggacgagaagctcttggatgtggcgggtggttatgaattgcgatctaaacttggacccgagcctgttcctggcggagttgtccttggcaaggtgtcggaatggtgggtgaaacgttggggcttcaatcccgactgtattgtcgcaccatttacaggcgataatccggcaactgtcgtggcgctttcttctcctggcgatgctatccttagcctcggcacatctaccacgcttttgctgtcgattccgccgtcggacacggctccgaaacgttttacgtcgtcgcacctgttggcgcaccccaccacttacgatgcacatatcgcgatgctgtgctacaaaaacggcgccctcgcacgagatcaggttcgtaataaatatgccaatgcagattgggcgaaattcaacgaactggtcgagcaaactccgactggtaacaatggtttgttgggttactactttagcttgcctgagatcattccacctaatgtcgtcggaaacttctacttcagcacatcgccctcgattcgtgcggttcctgaaagcgaggtttcggctgccgcgcaccctcgtgcagtcctcgaaagccaattcttgtctatccgttcgcgaatccaagccgtcttgccggctaatagcccaccacttcagcgcctcgtgctttccggaggatcctcttccaatccgattattcgccagctcgctgcagacgtgttcggtatgcgcgtttttgtcgccggccaggagggagccgcagagggcggcgcacagctcgcccgtttcgcttggtggcgtgaggagaacggtggtcgtggtacttttgaggaaatgcgcgcgttcgatggcgaagaagagcgcatgaaatgtgttgccgaacctaaagaggataatgttcgcgtttatgcaggcttggtggatgcgtatcgagaatgcgaggagcgagttgtcgaattgtgtgctcagacagcttaacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 46 - DNA, 5114 bp, synthetic construct
  source 1..5114: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactctggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatggagggaacaagccccttttttctcggccttgacgccagcacccaggctctcaaggcgtctctgctggacgttgatctgaatgtcttgtccgagctcgaaatccgctttgaccgtgatctgccccaatacaagactacgggaggtatctcggctcccgccgcggatgacgatcaaggtactgttgtggcccctgtgatgctctatgtcgaatcgattgacatgcttgcagagcgaatgcgttccgcttcgtggcctctctcgcgcatccgagccatctcggcggcaggacaacaacatgcctcggtttatttttctcgagcggcacctcgcatttttaccactctcagcgctgagaagaccttgaccgaacaggtggaacaagctttttcccgcaaggttgttccgaattggcaggactccagcacggttgacgcctgcaaggcttttgagaaagccgtgggtggcgcggagaagctcgcgcagatgactggttcgaaggcgcacacgcgttttactggcccgcaaatctacaagtttcgcacacagcaaccgcaggcgtataaggacacagagcgcatcggcctggtttcctcgttcattacgactttgctgtgtgtgggacaaggagaagatgaatcctctgtgattaaaggcatcgacgaagcagacgcatgtggtatgaacctgttggatatgcgccccgcgtctagcatcaccgcaactaaaggtcgtatcgaaccgggctggaatcagacgcttttggccttggcgagcggcgagtctgaaggatcggacgcttccctgggcggtgctgtggagttggagcgtaagctgggtagcatttaccgtgatgcaggtacacctgtcgccaagatcggctcgtggtggaccaagaagtatggattctcgcccgactgtcacgttttcccgggtacgggtgataacccggctactttccttgcattcagcctcgctgagcgcgaggctatcatctccttgggaacaagcgacacggtgatggtcgcaacccaccagtttgtgcccgatccagatttccacgcgttctttcaccccgcgcgacttccctccagcggcgacgcttttttcaatatgctcgtgtataagtctggctctcttgcacgagagtggatccgtgaccaatactgtaatagcgattgggcagccttcaacgcggacgtggagaaatttaagctgcaacaagatggtgagcaacgcgcaggcttttactggctccgaccagaaatcattccgcctggtgcatctggtatctatcgttacatcaagcccagcacctgtggagagtggcaaaaagtggacgacttctccgataagggaatgaacgcgtcgggaatcttggaaacccaaatgctcaactaccgataccgatcttcgtctatcgtcgcaggtccgacgacctcgagcggctcggcagtcgatacatcggtcctcccgctccaggccatttacgcggttggcggcgcctcttctaatccaaccattacacaaacgatggctgacgtgtttggatgtgatattgtgaaaccagtccaacccgcgcaagacggtaaggaatggaccccagccaactacaacttttgctcggttggcgcggcatacaaagcagcatggggttggtcgcgtcttcagcttaaggcaggagaaaaggtcccagaatttgacgagtttgtccatacaactaaatcctctcaagctaaacgcgcgggctctgtccgtgagggaacaggcgaagttaagggttctgtgcaagtgatctgccgacccactgaaggtcgaactcaggtctacactaatgtgatcggagcgtggaaacagctggaggaacgtgcgcagaaggaacagtaacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 47 - DNA, 4745 bp, synthetic construct
  source 1..4745: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactctggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatgtacatcctggcgcacgacttgggaacaagcggtaataaagccactttgtttgacgagtctggcctcttgatcgcatcgcgcacggccgcgtatcctacagactacgcctctggcaatcgtgcggagcaaaatccccatcactggtggaaagcaatcgtcgacacaactcaggcgctcttggagcttgtttctccgaacgatatcgcgggtgttgcgctgtctggacaaatgatgggctgtttgtgcgtcgacaaggacggcaaccctctgcgaccccatatgctgtactgtgatcagcgctctcaagaagaagaggcgaaactcacagagaagatcgatcctctccatttctatgaaatcactggtcaccgtatttctgcctcctatagcgttgaaaaactgatgtgggtgaagaagcacgagcccgaaatctttgctcagacggcaaaaatgctgaacgcgaaggactatatcaactaccgactgtgtggcaccattgcaacggatccctcggacgcttcgggcacaaatgcgtatgacctgaatcgctggcagtggagcgaagaaattatcgaggccgcggaactcgatctgtcgctctttccagaagtgcgttcttctatcgatgttatcggagagattacaaacgaggcggcgcgtgaaacaggtttgttggcgggaacgcctgttatttgcggtggaggtgatggatcctgcgcaggagtcggcgtcggctgcgttgctcccggtactgcgtataattatctcggttcttcctcttgggtggcattgactgttgagaaacctatcgtggacgaacagcgacgtacgatgaattgggcgcatgtcgttcccggcatgttgcacccctcgggaaccatgcaagctgctggcagcagctataactggatgatcaaccagctttgtcagaatgagcaggcgctcgccgcccaatccggtcgctccgttttcgagctgatcgacgagcaaattatcgcaagcccgattggagctaataaattgctctttttgccttatatgctcggcgaacgcacaccgcgatggaacgtggatgcgaaaggagcgttcatcggccttacattgggtcacaagcatggagatatgttgcgtgcagttatggaaggcatcactctgaaccttggatttatcatcaacatcttccgaaagcatgtgccaattgaccgaatgaccgtcatcggtggatgcgctcaaaatccggtgtggcgacaaatgatggcggatatttatcaggcagagatccgagttccgaactacttggaggaggcaacgtcgatgggcgcagcgattcttgcaggaatcggcgcgggcgtctttccggacttctccgtcattgaccgatttgttcgaatcgagcaaactgtgcagccgattccggaaaatgtcaaaaaatacgaggcgtggatgcccgttttcgaccaggcgtatcatgctctttgtgagatgtacaccgagatcgttaaaactgagctctaacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 48 - DNA, 5099 bp, synthetic construct
  source 1..5099: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactctggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatgacaacaacaccgttcgatgcacctgacaagctctttctgggctttgacctgtctactcaacagttgaaaattattgtgacggacgaaaatctcgctgctcttaagacctataatgtcgagttcgactcgatcaactcctcggtccagaagggagtgatcgcgattaatgatgagatttccaagggtgcaattatctcccctgtttacatgtggctggatgcattggatcatgtgtttgaggacatgaagaaagatggtttcccttttaacaaggttgtgggtatttccggatcctgtcagcagcacggctcggtgtactggtcccgcaccgcagagaaagtcctctcggagctcgatgcggaatcttctctttcttcccagatgcgaagcgcgttcactttcaaacacgcccccaattggcaggaccactccactggtaaggagctggaagagtttgaacgagttatcggtgctgacgccctggcggatattagcggctcccgtgctcactaccgtttcacaggtctgcaaatccgaaagctctcgactcgcttcaagccggaaaagtataaccgaactgcgcgaatttcccttgtctcctcgtttgttgcctccgttcttctcggtcgtattaccagcattgaagaggccgatgcttgcggtatgaacctttatgacattgagaaacgagaattcaacgaggagctgctggcaattgcagctggtgtccatccagagctcgatggagtggaacaagacggagaaatctatcgcgctggtatcaacgaattgaaacgtaagctcggaccggttaaacccattacatacgaatccgaaggtgacattgcttcttactttgttacccgttatggattcaaccccgactgcaagatctatagcttcaccggcgacaatctcgcgaccatcatttccttgcctctcgctccgaatgatgcactcatttctcttggaacctccacgactgtgcttattatcactaagaactatgctccttcgtcgcaatatcatcttttcaaacaccccactatgcctgaccattatatgggtatgatctgctactgtaacggatccttggcacgagaaaaagtgcgcgatgaggtgaacgaaaaattcaatgtcgaagacaagaaaagctgggacaagtttaatgagatcctcgacaaatccaccgactttaataataagctgggtatttatttccctctgggagaaattgttccaaatgctgccgcgcagattaagcgtagcgttctgaatagcaagaatgagattgtggatgttgagctgggagataagaactggcaaccagaagacgatgtgagctccatcgtggagtcgcagacactctcttgccgccttcgtactggaccaatgctctcgaaaagcggcgacagctctgcgtcgtcgtctgcttccccgcaaccagaaggtgatggaacggatctgcacaaagtgtatcaggatctggtcaaaaaattcggtgacttgtacactgacggcaagaaacagacgttcgaaagcctcacagctcgtcccaaccgatgttattacgtgggaggagcatctaataacggatccattatccgcaagatgggttccattctggcaccagtcaacggaaactacaaggtcgatattccaaacgcgtgtgcgttgggcggtgcttacaaggctagctggtcgtacgaatgcgaagcgaaaaaggagtggattggatacgatcaatacatcaatcgcctgtttgaggtcagcgacgagatgaactcgtttgaagtgaaagacaaatggctggaatacgcgaacggtgtcggcatgttggcaaagatggagtctgagttgaagcactaacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 49 - DNA, 5030 bp, synthetic construct
  source 1..5030: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactctggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatgttgtgctctgtgatccaacgacagacccgtgaagtctccaatacgatgtctctcgattcttactaccttggtttcgatcttagcacgcaacaacttaaatgtcttgctatcaaccaggacctgaaaatcgtccactccgagactgtcgaattcgagaaggacctgccacactaccacacaaagaagggagtctatatccatggagacaccattgaatgtcctgtggcaatgtggctggaagctttggacttggttctctcgaagtatcgtgaagcgaaatttcccctcaataaggtcatggcggttagcggctcttgccaacaacatggtagcgtctactggtcgtcgcaggcggagtctctcctggagcaactcaataagaaacctgagaaagacctcctgcattatgtgtcgtcggtggctttcgcgcgccagactgcaccaaattggcaggatcatagcactgcaaaacaatgccaagagtttgaagagtgtattggcggacccgaaaaaatggcacaactgactggttctcgtgctcacttccgttttacgggcccgcaaattctcaaaattgcccagctggaaccggaagcatatgaaaaaactaagactatcagcctcgtctctaactttctgacatctattctggtcggacatctggtggagttggaagaggccgacgcatgtggtatgaacttgtatgacatccgcgaacgtaaattctccgatgaactcctgcacctgatcgactcctcctctaaggacaaaactattcgacaaaaacttatgcgtgctcctatgaagaatctgatcgctggtactatttgtaagtacttcattgaaaaatacggctttaacacgaactgcaaagtgtccccaatgactggagacaaccttgcaactatctgttctctgcccctccgcaaaaacgacgttctcgttagcttgggtacctccaccaccgtccttttggttacggacaagtatcacccatctcctaattaccacttgttcattcatcccacgttgcccaaccattatatgggcatgatctgctattgtaatggttctctcgcccgagaacgaatccgtgacgagctgaacaaagaacgagagaacaactatgaaaagacgaatgactggacgctctttaatcaagccgttctggacgattctgagtcgtctgagaacgagcttggagtctactttcccttgggcgaaattgtgccgagcgtgaaggcgatcaacaaacgcgttattttcaatccaaagacgggcatgatcgagcgagaagttgcaaaatttaaggataagcgccatgatgcaaagaatatcgtcgagtcgcaagctctgtcttgtcgagttcgaatcagcccgttgttgtctgactccaatgcatcctcccagcaacgacttaacgaggacacaatcgtcaagttcgattatgacgagagcccactgcgagattacttgaacaagcgcccagaacgtacatttttcgtgggaggcgcgtccaaaaatgacgcgatcgtgaagaagtttgctcaagtgatcggtgcgacgaagggaaattttcgtctggagacgcccaactcctgtgcgcttggaggatgctataaagccatgtggtcccttttgtacgattccaataagatcgccgttccattcgataagttcctcaacgataattttccatggcatgtgatggaatcgatctccgatgttgacaacgaaaactgggaccgttacaattctaagatcgttccactgtctgaactggagaagacactcatctaacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 50 - DNA, 4046 bp, synthetic construct
  source 1..4046: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactctggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatggctcgcccgactgtcttggtggcatcggatcttgaccgcacactgatttactccgccgccgcgctggcgttgaccatgcctgacgcacgagcgccccgattgctgtgtgtcgaagtccatgaaaacaagcccttgagctatatgaccgaaacagcagcaggattgttgacggaattgggagatgcggccgtgtttgtgcctacgaccacacgaacacgaaagcagtaccaacgaatcaatcttcccggcccagtccccaagtacgcgatctgcacgaatggcggccaactgttggtcgacggagtgccagaccccgactggcacgacgtggtcacaggtcgcctggcagatgagtgcgcacccctcgacgaagtccaggcacatctcgcagaaaccgctgatccagtttggttgcgaaagcagcgcgttgcagaggatttgtttgtttatctcgtcgtggagcgcgaactcctccccgaagcttgggtcaaagagttggccgtttgggctgagaatcgcggctggactgtctctctgcaaggtcgaaaactgtatgcagtgcccaaaccattgactaaatcggcggcaatgcgcgaggtggcacgacgcacgggcgccggccttactctcgcagctggagatagccttcttgatgccgatcttctcctcgcagcagatcgtggctggcgacccggacatggagaacttgcagacgctgagtgggcggcccctgccgtggcggcgctttcggaacgcggcgtcttggcaggagaacgaattttgcgagagtttctccgaggtgtccgagaaggataacatggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgcagcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 51 - DNA, 4414 bp, synthetic construct
  source 1..4414: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtccttgacgggttcaatattcaatactcagtgttcaagcagtggtaggttgctccgtggatggaacgacgggtttaatttcttccgccatgcaccatggtccatgatcggttcagatggacagttggtatgagaaagtggtaacagataaatagaatttcgtacaagttggcaagagcctggccgatggtccatggctcgatggctcgatggctcgatggctcgatggccacacaccatgtcatctactttctgtgcttctatgcggacgagtcggaacagctgtcaaactgcgtttggggtatttgagcggaatttgaaaccttgttgaaactacattgtctcctcgttacttctctggcatctgtttgatcgaacggcgtcatgcaatagacacgcgatgaggcccaacgacggtgacgttgaacgcctggcccacctgctacaacaccgtagaacgtgttgaagttgaacgtcggacgttggacgttgagttgacgcgtgatgctggaaccgtggatgtagcagcgacagacaagccctctaagcaatctagtcgattttcaaaccaactaaacgagccttagttcggacaaccctagtccagtgcgggggcagcgtggggtcagttgccagcgaaggtcaccccagctagctgggttggctagcttgactggctcagaagtatcgatgctggagcttcaggccagtacgaaaatacactgtcgtagtgctccttccggagttctgggcttctgagcccggcggttgtgcatcggtgcacagtgcacggaccggcgggtgggacaacaaactgtctgggcgtgaaatcgtgcgagtgttgatgcaaaccggacgtacccagcacgcagttcccagtactcagacatactcagtacctcagtggttgagcctcagcatcgcctccgtatttctacaaagctgttgagtcgccttttgactttttactaccaccatcccctcttcttctaaaccccttacattccaaaaatggccagcgacaatttgtcggctgtgttgtacaagcaaaacgatctgcgcctcgaacagcgccccatccctgagcccaaggaggacgaagtccttctccaaatggcttacgtgggtatctgcggaagcgacgtccattactatgagcacggacgaattgccgacttcatcgtcaaagatccgatggtcattggacatgaggctagcggcaccgttgttaaagtcggcaaaaatgtcaagcatttgaaaaaaggcgaccgagttgcagttgagcctggtgttccttgccgacgatgccaattctgtaaggaaggcaaatataacttgtgtcctgacctcacattttgtgccacgcctccggatgacggaaatttggcacgttactacgtccatgcggctgacttttgtcataaacttcctgacaatgtttctcttgaggagggagcccttttggagccgctttctgttggagtgcatgcatgccgacgcgcaggcgtccaattgggaacaacggtccttgtcatcggcgcaggcccaattggtcttgtttccgtgctcgctgcgaaggcgtacggcgccttcgttgtctgtaccgcacgctcgcctcgccgcctggaagttgcaaagaattgcggagcagacgtcactttggtggtcgatccagccaaagaagaagagtcgagcatcatcgaacgtatccgttccgccattggagacttgcctaatgtgacaattgactgctctggcaacgagaagtgtattacgatcggaatcaatatcacgcgcacgggcggcactctgatgctggttggcatgggaagccaaatggtgaccgtcccgcttgtcaacgcctgtgctcgtgaaatcgacatcaagtcggtgttccgctattgtaacgattatccgattgccttggagatggtggccagcggacgatgcaatgtgaagcaactcgtcactcactcctttaaattggagcagacagttgacgcttttgaagccgcgcgcaaaaaggctgataataccattaaagtgatgatttcgtgccgccagggctaacattcaccctggagtaactgcgcttcatcgtttctttgttacgacaataataccacgctgcatgctttgaattgagtatatattttgatgactggttcaaatagatctttcctccatagccttcgagtactcgtgatctcagcattatcagatttgtgtccaggcccccgaggacgatgcggttttcctggagtcactagttgggaagatttgtactggttgggtgtaggtagagagaaccattgattttggtgtagctgttcgttttataaggccgagattacgtaatgcacatgctgccctagcaaatcgcgtcgcgtcacgcgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 52 - DNA, 1002 bp, Moniliella pollinis
  source 1..1002: mol_type = other DNA
aacacgtagaacacagggatcgccatttgtccgaaatgcaatctttgctctcgaagcttgaagcttgaatagcttgaagcatgagtgcatgaggggctgctatgatcttccaaccaaacttgcttgggcattgcactttggctggatagctatcatttagctgacccccaaagagactgccaatgatatgatatttgtgtaggtgcacgagtggatgagtggcgcgactacctgtcacaagatagatgctcgatgcacttcatgtgaagtggctagtggatgtccggagactggagtacagtaaagagtttggagttgggaacagcgcgaatgaaagaggctcagagagaggtaggtttgatgtgacgtcaagcgggctagtcagttagcaagggagtatggaatgcagcgcgcaacgcgcagcgcggagcccagtgcgcctgcgctcaggtactcatactcagtactcagtactcaggcgccatgccggagcttacatgcaatcaattaaccctgacgtctggcgcggcctgacgcctgaggcctgaggcctgacgcggccaagctccaagcgcaaccccaggctggcttgcgctggcgggcgcgagacgcagcagggggcaagtgtcgcagagtcgccgatcggatctgtggaacagcacaggaggcccgtgcggtcggctgcgcagtgcggtggagaagctggaagctggaagctggaaggtggaacctggaacttggaacttggaaaggaagaagagcgggaaggcgatgcacgatgcacgatgcacgatggggttggattgcgtacgagtagccgcacagcgcacagcgcacaacactcaccccatgcccccacgccccagcccgtgctcgggccatgagctgctggcaaccccccgaggcagccggcctttaactcaagtagcattcgccgccggtcggttcttcttagcctcccaccaccaccacttgcccgtgcttggacgctaattacaataa

SEQ ID NO: 53 - DNA, 679 bp, synthetic construct
  source 1..679: mol_type = other DNA
agctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatccatcatgcatcatgcatcatgcatcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcacc

SEQ ID NO: 54 - DNA, 1002 bp, Moniliella pollinis
  source 1..1002: mol_type = other DNA
catcgttccagcttgccttgggtgctagccagcgcaaaagtccgacgaacagacgaattgaccgtagtcgatgacagtcgatgacagtcgatgacagtcgatgacagccgacgaccgacgaacaacaaccaaccgcacgcagcatgaggccgagaggctcaggctgagccgctaaggcgctgaagcttgaagctcggtggtccagtcagatctcatgatctcacaccgccgcgctgctgcggtcgtatggttcaatttgctgtgcctagcctagcctgggtacgtcggtaactagccactagccactggccattatttggctactgggcacactaggaggcgcactagtccatcgcaagcggtaacttacctaccggccactacctgatgatttccctcttgcctggcggcctggcggcctggcggcgcactctttgcgctcggacccgcaaattgcaaaccaacaaacgactagtgtccggtacgtgtgcccaagtaacctgggcagacatagaaccacattgatcgtagggaaccgcaatggaccagtacgctattctcagtacattctgtacacttttgcctagattggaatctggtgaggatcaagatcaaatttcaaaccaactccacggcactggttcccgacacttggactgtagtactgaggtactgaggtactgaggtactgaggctcaactgagtctcaactgaccgtgaccgcgcttgtgtggtggcgtggattctagtgttggatgtgcgcagtggtgtgtgcggtggtgcgttcagattcgtcggtagaacttgactattcacgtcttgtgctgcgctgacgcttggaacctggaatgttaaaccttgggtcctgagggtctccaccccccaagtcgactgggttctatataaagtcgggtaggcaattggctagggagaatctctcttttctctttccccagaacacctgttcactcacttctcacctctttacttacctacttcaca

SEQ ID NO: 55 - DNA, 1000 bp, Moniliella pollinis
  source 1..1000: mol_type = other DNA
cctcgttcttgattgctttcaaaagtaatttaaggagagaacctggtcgtttgttgctctactgaatgatcttacctataccagcatacgacaccttgctgcccctccccttcaccaccaccatcaccactttcctctttttctttgggtttttctagtcgttgcatttatggttgattcgctctttgagaaccgtatggctgttattccttcccaactcttttgtctgttttatcgttgcatccaaacgtttgggctcttcgtacctcttttttctctgtttgccttttatttttggtttttctatccgtttgatggttgtgactcttgttttcctttgggaaggtttgagctcaacttctcttcggtccttgttggagtttgacttccttttctttttcagctgtggccgatcattacgatcaataatttgtgaatatgatatcgactacatcctgaatcttttagcgaaccatagcagtttagcgtcatagatatgcatgcatgatgtacatcttacacgcaccacggatgatgctcagcgctcagccacgcagccgctcagccgctcgctcggtacgctcggtcatgatgtgcatgatgcaagcctaatgattgacgggtacttgatctgtgtcccaattcgatcgagatcgatctcactatagtttgtcgaacttcgatgcacatcgagggtgcggaaaaaagctgacggcagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaaccctgccggccctatataactagtgcttcatcttcttgtcattcttgtctctttaccaacacaccggtactctttcacc

SEQ ID NO: 56 - DNA, 1007 bp, Moniliella pollinis
  source 1..1007: mol_type = other DNA
cttgacgggttcaatattcaatactcagtgttcaagcagtggtaggttgctccgtggatggaacgacgggtttaatttcttccgccatgcaccatggtccatgatcggttcagatggacagttggtatgagaaagtggtaacagataaatagaatttcgtacaagttggcaagagcctggccgatggtccatggctcgatggctcgatggctcgatggctcgatggccacacaccatgtcatctactttctgtgcttctatgcggacgagtcggaacagctgtcaaactgcgtttggggtatttgagcggaatttgaaaccttgttgaaactacattgtctcctcgttacttctctggcatctgtttgatcgaacggcgtcatgcaatagacacgcgatgaggcccaacgacggtgacgttgaacgcctggcccacctgctacaacaccgtagaacgtgttgaagttgaacgtcggacgttggacgttgagttgacgcgtgatgctggaaccgtggatgtagcagcgacagacaagccctctaagcaatctagtcgattttcaaaccaactaaacgagccttagttcggacaaccctagtccagtgcgggggcagcgtggggtcagttgccagcgaaggtcaccccagctagctgggttggctagcttgactggctcagaagtatcgatgctggagcttcaggccagtacgaaaatacactgtcgtagtgctccttccggagttctgggcttctgagcccggcggttgtgcatcggtgcacagtgcacggaccggcgggtgggacaacaaactgtctgggcgtgaaatcgtgcgagtgttgatgcaaaccggacgtacccagcacgcagttcccagtactcagacatactcagtacctcagtggttgagcctcagcatcgcctccgtatttctacaaagctgttgagtcgccttttgactttttactaccaccatcccctcttcttctaaaccccttacattccaaa

SEQ ID NO: 57 - DNA, 1000 bp, Moniliella pollinis
  source 1..1000: mol_type = other DNA
tcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaaca

SEQ ID NO: 58 - DNA, 997 bp, Moniliella pollinis
  source 1..997: mol_type = other DNA
tcggaccccaagctctctagcttcaagctttcaagctagcttccagccagggtcatggaaccgtaggagctgggaaacgtgacgttgtcgctatgcctatcccatataagaattaacgtcgaatcatgaggcgcatgggaggctgcattggtggtatgatacataacgttatgattctatatgagcgatggagcgcggccgtctgccgctagtgtggaagttcgatacgcccagtccatttcgttcagatgacgtttggtccagaagtacggtacataccgagcgcaaacgtcgaacttatggccttggtcgctcgtggtccggcgtcgtcgacactttcatcaacggacgaaaggtagagccgaacgccagcggccaatggcgatggcgatagtagaatgaatagtcgtcagatctaggtcttaggctacaggccaccctgcctgaggcctgggggtcttacagcgagtctaaaaaacgaaaagccaacgtgccaacttgggcctgcccatgcaccctgccagagagccccaagctggtctgatgcaagcggagcaaaatgttggaagaatagtttcaacgatggtttggttccagtacgcacagtcgagagatgaggtactgcgcaacgagttgttggcgacggaaccgaagatggattgaactatttgatctacaggtgatggcgtggagtcgggcgtggggccgctgtgtgatatgtagaagtggcgctattcgagggctgcgcccgtctggatggatgcagaatgaagcttgcaggccgcagctggcaggatgggcgcagaacccggtgaaacatagttaaagcgcggggccagtgtcatatttcgcttgagccattctactcgttggcagcccaggcgccgctaatttacgccaactcttggtactcgagagctcgggcaatgtggtatataagcttgaggttccctacagcaaagcctggcttcttcaccacaaccgcactctagcaaac

SEQ ID NO: 59 - DNA, 1002 bp, Moniliella pollinis
  source 1..1002: mol_type = other DNA
gagcgtagagagctccatcgatggcctggttacaagttgcctgaagaccaccaatacggcccgcacgccaacggcaggccaagaaaggataattgacaaccgtgaatctgaaagcttcggcgctcaggagctcatgtgttttgttgtagatataaaggtttctgaataccagttgggtattgacacggggagttggactgaatctctcagatctcccagatctcaagtactgagtcccaagcgtgccataattaactctaggttgcgcgggacacgccacctgagatcagggtgtacgcgcatactaatccattccaccaccaaagcaagactgagggtaccgcctgaggttctggctattccaacagtcacaagtcaccgcgattgccgtcaaattccaaagcgtagtaggcgctcgacggagggatgatggccagtacccaatttcgagcactcagttagatctgttcgtgtttagttgctcctgcagatctatcttaaatgtatactgtgccaggtaggatctctcggcacctatagatgatataaaagatatgatgatatgccaagtgccaagccgtcaaactgacaaaatgaaagaaacagcaggcaagctaggcaagcgtctatcgggggattaaacggatgagtcagcagcacaggagcagtcagtcgacaattccggctttttcccgcccgcaaggccccagaccccagaccccagccgccaccatgtccgtatccgccggggttagcatgaccacggcagatttctccgctcagcttgctcggacgactatatataaagaatcctttcctcgcagtccgtccgagcccttcctccaccccatcggttaccaccaacgacatcacttggtcttgacgccgcgctgcgttttcccgcttccgcgtctccctagtgatttagctggtgttttcctctacccgccactctactgtttcatcgcttgttgccaagttttctcagttcattctcatcaa

SEQ ID NO: 60 - DNA, 1002 bp, Moniliella pollinis
  source 1..1002: mol_type = other DNA
tggacgtggacaagcaccctgaagtcgctcagggcttccagatcaaggctttgcctaccgtctacggcttgtatgagggcaagcctgttcgtcagtgtaagttttaaaatatattgttcttgtctgttcgggttttacatcagtggtgtactgacacggctttttgttttattgctgcagtccaaggtgtacttcctgaggatatcctgcgccaatttgtccaagatcttcccagcttggccgcccaagatgcccagaacccgcctgctgaaagtagcgaggctgccaactaagtatagtccatcctatgagctgtcctaccccaaacaactttttcacgcccaacgtccaaacgtcacttcttattgcggcaaagaacagctgcttccccactcattcattacccctttctgatccacgaggtgtacccatggcctttgtacattccatgaaacaggtatccttttccccaccattacatctgaacttacaaagactttgcttcctcttgtacacagctcgtcgtccatcgtccatcatcgatgacgatcatcctgcattcggtatcatgcaccatcatgcagcgatggcaacgagggccatgcaccatgctgcatatactatgtgtatagccaatagccaatcttcaatctccaacttcctacagacacagcctgtggggaatcgcttaatcgctgtccatgcgtccatgctccgcgttcctcgcgctcgtcgcggcctcgcgacgattaagtcacagtcacgcccacacagcgcgcgagcgcacgagccaccatcatcacgtgatgagacaaagagtccacttccgtaaaagcaatcttaataatacatgtaaccctagcaatgtagccctaataatttgtttggcctttattttattgcgcccccgcagcttttgcaccccggagagtaaaagtattccggttgctcgtcagagcatccctttctttctccatcagcaaagcgtgcgaccgccaagaa

SEQ ID NO: 61 - DNA, 1002 bp, Moniliella pollinis
  source 1..1002: mol_type = other DNA
tttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaa

SEQ ID NO: 62 - DNA, 921 bp, synthetic construct
  source 1..921: mol_type = other DNA
tttggatgaggcattcaatgtctccatgtttgaagaccagagaaagtgtaggtaacacaatagccatggacaattagcgaatccgaatatgtgtactaacattgtgcctccaactgacagctggtcgaataatgattgaaatcatcaaacatatcagagatatatcccagaaccatttaacaactgttattgtatgtatgcattactgttttacattgtgtctttccaaatactaacattgccattatccaatccatacatatatatatatatagattacacaacgcaaacctcgacagtcgattggtttacatcgtgtaagcagccttttacggaatatgatggacaaccagatcctcatagatggagacaatgtgcagatagattccatttatgatgacattgatgtgtttttaaatgcaaaataatctcctatataagtgagttccgagtctccccatgcttatcttatcgtgttttttctgtaaattaggtcaaagtattcaaacaattccctcaatctttcccattttttgctgagcccccatatcatcaaattaacgtatcaaatttcctaattagtagccccgtatgttaaattagacatgtgtgactgccatcccggacagcctacccaatgacacccgcgcaccgcacatattgtgtttagtgcgcgccgtctgctgaagcgactccctgtttgggaggaaccgagggcgggttgcccgatcccttgcccctcgctcctcctcctgggctcccccttgcagagggacaccgaggggatccctcgtgtgagagcttggaggtggatggtggtcaattttctcatttgattgaaagatttgttatattgaaaattcagtttgtggaagttgtgattaaaaggttttactgtttgttctgtagacacattcaata

SEQ ID NO: 63 - DNA, 500 bp, Moniliella pollinis
  source 1..500: mol_type = other DNA
acggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcct

SEQ ID NO: 64 - DNA, 500 bp, Moniliella pollinis
  source 1..500: mol_type = other DNA
gtactaaccaagtctattgtaaagctggtgtctgctgctgcttcagcaagtgccattgctaccatcgcaagcaaattgcaaaaaaattcgttcaatggtctaatttaagtaaaatactcataatttataaaatgccgcttgtccttaccttttgtctttctccttattatagtagctgtagttatcgtagtcctcaggacttcgtcaacgctcgttgatgatcatatatacttcacctcattcattcataaagcattcattaggattgtatgatccggcagtgcatcatgcatcatgcatcatgcatcaggacatcatgcatcatgccgtatcggtcgatcattgcatttgattggataatacgatcgtgtttaggcattggcagtcgaaacgaggataaagaagaggtcagtaccagtgcacagcacgcgctcgcgctctatgcgcactttgacgcttgaaaaaaacttttcagccaatcggatatagacagcggaccc

SEQ ID NO: 65 - DNA, 493 bp, Moniliella pollinis
  source 1..493: mol_type = other DNA
atggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgca

SEQ ID NO: 66 - DNA, 500 bp, Moniliella pollinis
  source 1..500: mol_type = other DNA
atcagttggaatcatcaaccttttttttttgtatcgatttcttcctaccctcacctcccttttcccccttaactcctcgctttgaacctccttacccgagtgcctacctttacctttttctttttctctctcccctgcctaatattctaggcttgctactatactgctctctctaacacgatttttgcgcttgtaagcatttttctctttttaaatctaggttttatcatttgttgcatttatccaagtttttttttttttttgtttgtagtggttttactgtttttgtgttttgtagcttgactaggttcttattctattgtagatgtatatgatcggatgcattcccgatctaaaaggaaaatgttctcgactgatatttatttttaccttattgtcttcctaaatggtttgactcttgccttcttccttgggacgctttgcaacgttttgtacgtacgtaagggttcatcgtttggataagcgacacagttgtcaag

SEQ ID NO: 67 - DNA, 324 bp, Moniliella pollinis
  source 1..324: mol_type = other DNA
attcaccctggagtaactgcgcttcatcgtttctttgttacgacaataataccacgctgcatgctttgaattgagtatatattttgatgactggttcaaatagatctttcctccatagccttcgagtactcgtgatctcagcattatcagatttgtgtccaggcccccgaggacgatgcggttttcctggagtcactagttgggaagatttgtactggttgggtgtaggtagagagaaccattgattttggtgtagctgttcgttttataaggccgagattacgtaatgcacatgctgccctagcaaatcgcgtcgcgtcacgc

SEQ ID NO: 68 - DNA, 496 bp, Moniliella pollinis
  source 1..496: mol_type = other DNA
atgtgcttagattaacatacacttgacttccttacgcgttgatatatacttggatctccataacttctattgtatagtttaatcaatggactccatgttgacatcccattatgcgatctatagtagctggaaatagcaaaattgaagttcggcagacgactgcaccaggaggggtagattctgtcccataactccctaccacaggccgtgctctggttcgatttaccttagcctgatgcgctcaatgtaacgcgtaattctgagtggggttcggtcgtccaaggtttcagtatgcgctgtgcagatagtactgtatcgtgaaactcgaccgttgcacatgcactttatcaagtgtcgtaggcccagagtcgtaggccaagagtcgtaggccaagatggaaggcctgaagaaaaagaaagctttgatcgttctgatctatgcaatggattcatgagctaagtattagctatacaaatgattcgagcatagcagaagg

SEQ ID NO: 69 - DNA, 500 bp, Moniliella pollinis
  source 1..500: mol_type = other DNA
gttgctccctttgctgttctatgatccaaaagatgcttagtgctccactctctactcccgactaccttttacgcattctgacatgactagtagtgcaatgttacccctggtgttttctcgcaatcactttctgtctgctatagcttttgatcgtcagtagttttttggtatagctaaagtagacttttgtacctggcgttcaatgtaattttactccttttttgtgcgatgcaacagatggtgtaaagatcgtcgttaaagttttggcttgatgcttgattcggtgacggttgcttgggtgatccttatatcgtctcctggtgttctcataagcccaggctcttcttagctatgtacaacaggtccaccactgactatgttatgtagaggcatgcatttaacttacggaaagatttaatggagaagaacgtacctagtcagatgcttgacatgaaaacgtattcctgcaattcacaaatcagtatcctgcctgtccactc

SEQ ID NO: 70 - DNA, 500 bp, Moniliella pollinis
  source 1..500: mol_type = other DNA
gcggttcgcacttttatcccaggcatgaaaaatgacgcacggctgtcgcacgctacacccagtagtcgccttttcaggcagtctatgaggggagcttttcattgcccaaaattactagcctgcaattcatgatatcaggttcactttctcccgatgtctatagatttctcatagcgctttgaaaagacaatttagctactcagtttataagcaaatgatgtggaggtatttattaggtacaagatacgataacgtttacacgcggggaagcaagtggcaaatcgtcgacaacaccgcatggtggtggtggaatcgtactccatcaaagcttgcttgtcactttgggaggcaccaggttgaggccatcgcccgaaggtggttgtggccagggtttcgatatttcatgaatcaacgtgttgatggtattggattgggtaagaggtcgatccggctgttggtcagagtgctcatcacattcgtcgttgtcaagcccgagttgc

SEQ ID NO: 71 - DNA, 500 bp, Moniliella pollinis
  source 1..500: mol_type = other DNA
gtggcgttgattcgtatcttattattgatctgtcctgttccccacgttgccaagtgattgttcatgctatcctctctccattctgtttgtgttgcacagtcggtagttgtacgtagtagctcgtcacaaaatatgaagtgagccttgcaatgtaggcattcacaagctgctaggcaggctatgatctaagctaagtggcaaaaattctcttgattcaatgccaaactatactattgttcctcaggcaaaagcagctatataagttgcattcaacgcaaccgccatacaagcaaattagagagaaacgggatataaggtgctttatccctgaaggagtagccagcgtctacttggttgctacacaatacagcaaacagtgtacttaatgagccattggctgaactggagctcgcagaggttcgagaagggcgggtcataccagctgccctccatggaagctggggtgtcgatgatgaaaagtgtcggaacgtacgctagta

SEQ ID NO: 72 - DNA, 500 bp, Moniliella pollinis
  source 1..500: mol_type = other DNA
acgaagggtttcccatctgtgtgacctgatccctttttacgtttttttgttttgttttttcttttgttcatgatcaacttgggagtgattcctcttggtgatgtcgaaaattgaactgttataccaatattctgttgcctttgtttcctgtttgtagtttactactttttacagtagtcgttgttcctgccttgcttgcgtttgtcagggtttccctttagcttagtaccaatatctagccttaattttgcttattctttctcaccgcccatgattgatttcaatcatgattaatgatttatgcatgaaaactcaaaatactcaattgtcttgtacaaggcaatcacaagctggtcagccattctcagcgcctataagagctttcagccaggccggccccaaaacgatctaccctgtggacctggctaggctaaactttggaaagtcatgcaattatgaacgtttgaagctcaagcgctcctaccctctcctggagcagt

SEQ ID NO: 73 - DNA, 500 bp, Moniliella pollinis
  source 1..500: mol_type = other DNA
tggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgata

SEQ ID NO: 74 - DNA, 500 bp, Moniliella pollinis
  source 1..500: mol_type = other DNA
gtttcttggctgttgacgaaatcttatgaaatgcacgggtctaatatttcctgccaattgtgtacccatctcgtttgtgaatgtcatagctagctctgtagtttctaaacgaccgtcttaggccggggtttgctgattggggattaaggataggatacttggccagtttgggccgatcaatatcaatctgctaggcgttccttaggtcaacttgaattatgatatatgaggtatgaggaaagtgggctaaatctgtggattaggatcagaaaaaacgtggcagaccctcgatctaggcctacgtcagcaatttcagcaatttcagcgccttgccccttagataggtttcagccgctcagcgcgcgcaatttcagcatggatgagatcaaaccaaggggcagcagttggcgaccctgtagttccaatcgcatgcttagtgcataacgcgcacatcaacgtgtcagattctcagagatctcaacaagcccactagaccctag

SEQ ID NO: 75 - DNA, 504 bp, Moniliella pollinis
  source 1..504: mol_type = genomic DNA
aatcgcctgtgtagcgaacggggccagctgggtaaggccagcggccggtagctaggactatgaggctaccaggctgctgggtaactaggcagctaggcacctaggcaactgagcaactgagcaactgggcaactgagcggtataggtttggcttgggcacagttgatattgatatcaaccagtcgctcgttcggttcaccagaacccagtgtgtggtagcgagcgtagaatagccaaaaggttttgagctgaggcgctgaggcgccacgggaggacatcaccgcctgagcacacaggatttcaaactgtcagacgggcgtggtgccagtgttggaagtgggcgaagcacctcgcatctaagacatgtaattaaactctcgacagccatcgtccattgacaccaacgtgcaccaacacacgcgagagctgcttcgttgtgcctgtcgacgacattgcctcgttccccagccaagtcgccccaccgctacaccaagcgcactcgca

SEQ ID NO: 76 - DNA, 493 bp, Moniliella pollinis
  source 1..493: mol_type = genomic DNA
cctttgcctcacgcctctcgtttgcacgcttcttcacggcttatcaccgcttattatatccatggaatcccctccaaagttttcttcaacatagcttcatatcttctaccataacatctatcccaccaagtagcacatgaattcaacgtagcttgagaacttttcctcgtcagctgattgcgttctttaggcagtttgcttctaccccaattgcttctaccccaattgcttctgcttatttcaccagtccgctcggagatctctgacaactctgagatctgatgatcaacctcttgtgcaacttgtaaaatttgatcgatgagacgattagggacttgaagctttaagttagacgcgatgggcggtaatctatatgcttagactttgttgctatgtttgtcctataagatctgatgcatgatgtatcattacccagacgggctatcgcacaaggtgcagatgcatcttatcatgacagtgccacactgttggc

SEQ ID NO: 77 - DNA, 499 bp, Moniliella pollinis
  source 1..499: mol_type = genomic DNA
catccgaaattggtggtttgacaaatgcaccttcttctccaggggctcgtgcaaacgcagtagccgagaagaggcgtaccggagaggaagacccaacaaccactgatcggtttgaagttggatgagaaagttgtgccaatcgcggagccgtagcaacgctacgagcgctacctacaacgactccacgcgcaatcggtacgcgcaaaggaacggatgagcgaccgagcaggttgcgcgaagccatgacgattgctctacccccaacgagctggatcaagttgggcagaacataatttatctcgcttccccgcgccacaagtagctccaccattcttctcaaaatagcccgattccgctttcgcggggcaaaacttccgcctataccgatccgattgaaacgccaaaaaaattaagttgtagtacgcctgttggtcgagcccgactggagcaccatttgagacactaggagataatttattcttttccttcttggccccca

SEQ ID NO: 78 - DNA, 501 bp, Moniliella pollinis
  source 1..501: mol_type = genomic DNA
cctgaacgtcctgaactctgtcttattacataccattaatttccctgccttccctctctacgtcatcggaattttatctcttcctgagcagcaaagatttggtccttgtccgggatacaacggagagtcatgattgggtttatggaagagaggcgatagcacttgcattttgaccattcggggattaagcgaacccccaagtggcacaaagagctcgtatgagggtcagtaagtatagagggacatgcaaaggagagaaaaggaaacacaatattgacaaatagagacaagtacaggtgatcgcaatagcatgatgtaggggtatgcgcaagtcacaatcgttcagggcgggcataacatcagcgtacccgagcacaatggttaatgattaatgagcagaggaaaaaataatgattatgccggggatggcaggactaactggcgcagctcttcagcttcggcaacgcgtgcacatagtggtgtagcaccacgagattgagc

SEQ ID NO: 79 - DNA, 300 bp, Moniliella pollinis
  source 1..300: mol_type = other DNA
cgggtaagaccaagtcggttggtgtttcgaacttcagcactcgtctggtcgacttggttgaggaagcttcgggcgaacgtcctgcggtaaaccagatcgaagctcaccccttgttgcaacaagacgagttggttgctcaccataagagcaagaacattgtcatcactgcttacagtcccttgggcaacaatgtcgctggtaaaccacctctgactgagaaccccggtattgtggatgctgctaagcgtttgaaccatactcctgctgctgtgctcattgcttggggtattcaacgcgggt

SEQ ID NO: 80 - DNA, 400 bp, Moniliella pollinis
  source 1..400: mol_type = other DNA
aaccagatcgaagctcaccccttgttgcaacaagacgagttggttgctcaccataagagcaagaacattgtcatcactgcttacagtcccttgggcaacaatgtcgctggtaaaccacctctgactgagaaccccggtattgtggatgctgctaagcgtttgaaccatactcctgctgctgtgctcattgcttggggtattcaacgcgggtacagcgtcttggtcaagtcagttacaccctcacggatcaagagtaactttgaacagatcactctgtctgatgaggaattccaacgggttaccaacctcatcaaggagtacggtgagagccgtaacaacgttcctttcaattacaagccttcgtggtccattgacgtctttggtacccaggacgaggcta

SEQ ID NO: 81 - DNA, 453 bp, Moniliella pollinis
  source 1..453: mol_type = genomic DNA
gcgaggagtagttactaggtagcggtatagcaattccgcccataatgcataaccgttctccctaccaaaacaaaacaatggctacgtggccggcgtgcgtggccgacatggtgcgggtgcggggggcggggtacgagtgtggggagttcactagttcgctggctagctgactagctgaccggcgtgtggttgtggcagtacgcagtgggaaagattaaatatgaaatattgaatatcaaaatgacgtttggggtggcacgtgcgcgcgccaacaacaacaagagcagccaccaagccaaagcaccaagcaccaaccccgcacaccattttgttacacccgcggcagaaatggcgtcagccagcagtcactattactcaatattcaataggggttccacttgactcacttcctcatgtatatatatatccatctaatcttcccaatacataacc

SEQ ID NO: 82 - DNA, 567 bp, Moniliella pollinis
  source 1..567: mol_type = genomic DNA
gcttgctggtcacattattgtcagtacctaccatgaagaattcaatattattttacattgtcaaccattacatggatcccatgttgtgcatagcctctatgtcatcctttggtcttttatgtctgtagcgtgctagtagtattcaagttgtagcactgctgtgaaacagtacgttcaagtagtgtgcagcagtacgttcagttagtggactggggcagcctcgccttcagcaaaggttaggtacatgagccagttgctcactcttgctttctacgaatcccataggctactttgccccctaaccgtgacgaccccctgtgtttgagaagtttgagtctttttacgttaccgtgaccaagcgagcagagtagaaactcttgaagttgtgtatgtaagaatacaaattgagaacacaaggttacctacctttatagttggtaaacagcaagcaataccagagattttcatcgcaacgtgactctatcattcactatattaagttgcacacggatgttgtacaacactacacgtaaaagagagaaaaatactaatgtcgatcgctcaggt

SEQ ID NO: 83 - DNA, 500 bp, Moniliella pollinis
  source 1..500: mol_type = genomic DNA
gcggcctatgcagacgcttggagcttgggcacattggatattggatattggctggctgccgttcgagatcccgttgctgtcgccaccgccaccggtcacgtggtcacttggtcacgtggaccacctgcactgcgcttagctacaagagtgcatgatgcatgatgtcatgcatgatatgcatgatacaagcatgaatattgatttcaatattgaacatttagtattgatatctcgatatgcgtaacacgtgacacgcaccatatcaatcagccgcaccggcactaaatgtttcaatttcaacatcccacacgctcgaatgctccctcgagctaatcggatttcggagctccgacatggccggcctcgagcggagtttgaaactccgttccgcccgaccccgctggtcgccgcgatgaaatcccgagtcaagtcatagcaagccaagtggctggctgactcaaaagctaataaaagtcgtagcaagtaatactccgcacgct

SEQ ID NO: 84 - DNA, 500 bp, Moniliella pollinis
  source 1..500: mol_type = genomic DNA
aaatcttcatccagcgtagcaagcatatcgcgcatgtccgagttcctcgagtgtgcataccgaacccaaaaagccatggatgtcattgacctgctgtattcatcaatcgagatatctttactccggagcgtacagtggcagatattatgtaggagatgatgggacaggatgcagagatacataatccatttccagttaatagggccgctagatacttgatattagcctggcagcgagcgagcgagggtagagtagaatagagtccagtatagatcccaccccatggtgcgggaagtggatgacgtaatagcgcgatagctccccgctcccttgtcacctaggccacgggaccctacgtgattgcatcgtgcgtgtcgtcgcctgtcgacgcctggtgcaggcatacgctgcgtgcttcgacgatcaacaacaccaccaaccacgactccagttccggccagccagtaccatggcgtcgttctttgggtttagtgcaccgg

SEQ ID NO: 85 - DNA, 500 bp, Moniliella pollinis
  source 1..500: mol_type = genomic DNA
tcgcgtcttcaaaccgtggcgcgcgacacgtgccgcgaccaaaatttgttggtgtatgggaatagggttttctaaaattctgttgaggatgtgagtatcactggcatggcaccagtgtactgctcatcgataatcataaacgtatcaacccttcggctctgcgatctgtatctgctactggagggcacagcaagccttctgcttggttgcgctctgcgctctctgtggtgctttgccgctcgcggccaaggtccgcagcgggcattcaccaccatacgggttaagccaccagacgagtgctgctcgccgggccagctcggcgcctgtcacgcccccacattggcttgatgctcggctccttctttcatcttggaaccggcgggcaatggcgcttatcaccactcctgggccgggccccgcgtatactctcataactagtgcacgctttcaaataaatacctcgcctcctggctggccgaccgacgtttacccatccgccc

SEQ ID NO: 86 - DNA, 500 bp, Moniliella pollinis
  source 1..500: mol_type = genomic DNA
atcctgtgctcgtctcttacattcatcttcctgcccataaaactttcaagctatttcctccaacgtactcaacaacctatttttttgggccccgctcaaaacgcaccttattgcgtctaagtgtattctaaccacgcgacctgaattgcaaagcctgttacccccatcgcaccttcgtcttaagatagttgtttaacgagccttggccagttttagccttgaccatgactacagatcataatggttagatgcttcttgatcgataatgatatattgcatgtcgacgattgaagatttattggatgtcgatgacgactgtcgagctcttattcttagcaacaacttaaatattcactatcgaatgcacgcgacatcatgcatcgtgctcatgaatatcactggatcactctcgatcgcagcccactaggtcagcataacacgcttgaagctgacttgaagctttggaacaaatggcagcaacagtggcggtcacggtgacc

SEQ ID NO: 87 - DNA, 500 bp, Moniliella pollinis
  source 1..500: mol_type = genomic DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcg

SEQ ID NO: 88 - DNA, 486 bp, Moniliella pollinis
  source 1..486: mol_type = genomic DNA
attgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 89 - DNA, 499 bp, Moniliella pollinis
  source 1..499: mol_type = genomic DNA
tggcatgtaggcctgtaggggctatgcctatatgctcatgcactcatgtcgcgatgttgcgatatcgcgatggaccaaccaagcaagcgtgcagcgtgggcgcttgggccaagggtgctaagctaagtgtaagcactgaagtgctgaagtgctgaagtgctgaacgtggaacgtcaggatgacacatcctctcccttcggcttcgtccaagaaaggttggtcagccatctgagatctgagatctgaaatgaatataatgatgaattgagtcgatgagatctgactatcttgggagattgcaccaatgcgccacggtgaccagatggtcttggtcttagctggtatagtggcacctgatctgccttgacagatctcatgttcatacgttcatgcattcaatcttatcgtctagccgtctgcgatctgcccaactagacgacgacaaatgtaattgatccttgtaaccctgttgctggtcgcagcagcgcgatacgatgcc

SEQ ID NO: 90 - DNA, 504 bp, Moniliella pollinis
  source 1..504: mol_type = genomic DNA
ttgctgctacgacctcattgatcgatgatctccgacactttgagtagctttccatcctctctttactgtactttgtattcttgctactttgtgttctttcttaatggtcccgaaccggtgattttgctcatgatctttttttttttttttcgtcaactgcaagaagatgaaaaatctactgttcgatagcacctcataaccgttccagagtcccgacaaccacaaggatgtttcttagataggtgtcagcaagatccacaaaaaagtgtaggtattagatggtttcaagcaaggtacaatattctaattcaatactcatttgaataatcaatattcaacagcttctagcctacaagcttcaatgttctccgtcggttaactcagtatacttagtactcagttcaagtcagtgttgttaaaataaaatcacgtgatccaaaaacttgaaacttttcatttttctcctcagatgttggtgattcgtcccatagcggttaccacaac

SEQ ID NO: 91 - DNA, 1679 bp, synthetic construct
  source 1..1679: mol_type = other DNA
aagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttctgtcatgacttgatttcccacatctatgctgctgatggttctgccgacgtcatgagagacatttctgcctctggtgagccagccatccctaacattaaggacttgttgaacccaaatattaaggctgttaacatgaacgaattgtgggacactcatttacaaaagtggaactatcaaatggaatacttggaaaagtggcgtgaagctgaagaaaaagctggtaaggaattggacgctattatcgctccaattactcctaccgtcgctgtcagacacgatcaattcagatactacggttacgcctccgttattagcttattggatttcacctctgttgtcgtcccagtcactttcgctgataagaatattgataagaagaacgaatcttttaaagctgtttccgaattggatgctttggttcaagaagaatacgacccagaggcttatcacggtgctcctgttgctgttcaagttattggtagaagattgtccgaagagagaactttggctatcgccgaagaagtcggtaaattgttgggtaacgtcgtcactccataatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 92 - DNA, 1903 bp, synthetic construct
  source 1..1903: mol_type = other DNA
ataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtttccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataagcgtccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttaagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttctgtcatgacttgatttcccacatctatgctgctgatggttctgccgacgtcatgagagacatttctgcctctggtgagccagccatccctaac

SEQ ID NO: 93 - DNA, 876 bp, synthetic construct
  source 1..876: mol_type = other DNA
agatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctgggggcgtgagttcgcactccgagatccagctggaaattgtgttcatttcgtagctgaggaacaggattaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 94 - DNA, 1087 bp, synthetic construct
  source 1..1087: mol_type = other DNA
gaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatggcaaagttgacgagcgccgtgccggtgctaaccgcgagagatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctgggggcgtgagttcgcactccgagatccagctggaaattgtg

SEQ ID NO: 95 - DNA, 1049 bp, synthetic construct
  source 1..1049: mol_type = other DNA
attgaagcccttgatggatcattcaccacagacacagtttttcgcgtgacagctactggtgacggatttactctccgtgaagtgcctgtagatccgccattgacgaaagtatttccagatgatgagagtgatgacgagagcgatgatggcgaagatggtgatcctgactcgcgcactttcgtcgcatacggtgatgatggtgacctggccggatttgttgttgtgtcctactcggggtggaaccgtagattgaccgtcgaagatatcgaagtcgcgccagaacatcgtggtcatggcgtgggacgagcactgatgggtctagctacggaattcgctcgagagcgtggggcgggtcacttatggcttgaagttaccaatgtaaatgctcctgctattcacgcttatcgccgaatgggtttcaccttgtgtggcctcgacacggcattgtatgacggtactgcttctgacggggagcaagcgctatacatgtctatgccctgcccctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 96 - DNA, 1257 bp, synthetic construct
  source 1..1257: mol_type = other DNA
gaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggaacgactttagatgataccgcctaccgttataggacatcagtaccgggcgatgcagaagcaattgaagcccttgatggatcattcaccacagacacagtttttcgcgtgacagctactggtgacggatttactctccgtgaagtgcctgtagatccgccattgacgaaagtatttccagatgatgagagtgatgacgagagcgatgatggcgaagatggtgatcctgactcgcgcactttcgtcgcatacggtgatgatggtgacctggccggatttgttgttgtgtcctactcggggtggaaccgtagattgaccgtcgaagatatcgaagtcgcgccagaacatcgtggtcatggcgtgggacgagcactgatgggtctagctacggaattcgctcgagagcgtggggcgggtcacttatggcttgaagttaccaatgtaaatgctcctgctattcacgcttatcgccgaatgggtttcaccttgtgtggcctcgacacggcattgtat

SEQ ID NO: 97 - DNA, 1199 bp, synthetic construct
  source 1..1199: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 98 - DNA, 1475 bp, synthetic construct
  source 1..1475: mol_type = other DNA
ataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatccatcatgcatcatgcatcatgcatcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 99 - DNA, 1621 bp, synthetic construct
  source 1..1621: mol_type = other DNA
tattctggtacgagccatcccaaaaatggatcatgacagctgccaagagccaggactataagattgagatctacagcagcgatgacctcaagtcctggaagctcgaaagcgcctttgcgaacgaaggctttcttggctatcagtatgagtgccccggcctcatcgaggtgcctaccgagcaagatcctagcaagagctactgggtgatgttcatctctatcaatccgggagcacccgcgggcggctccttcaaccagtattttgtgggttcgttcaacggaacgcactttgaagcttttgataatcagtcaagagtcgtcgactttggcaaagactactacgccctccagaccttctttaacacggatccaacgtacggttcggcactgggcatcgcgtgggcgtcgaactgggagtatagcgcctttgtccccactaacccttggcgctcgtcgatgtcgttagtgcgtaagttctcgctgaacaccgagtaccaggcaaatccggagacagaactaatcaaccttaaggcagagcctatcttgaacatctctaacgccggaccctggtctaggttcgccaccaacaccacgctcactaaggccaatagttacaatgtcgatctaagcaattcaacgggcacactggaattcgagctcgtttacgcagtcaacaccacccagacgatctcgaaatccgtctttgcagatttgtcgctgtggttcaagggccttgaggacccggaagagtacctccgcatgggttttgaagtctcagcgtcatcgttcttcctcgaccgcgggaattctaaggtaaagttcgtgaaggaaaacccctactttaccaatcgtatgtctgtgaacaatcagccattcaaatcggagaacgacctgtcctactacaaggtttacggccttctcgaccaaaacatcctcgagttgtactttaacgacggtgatgttgtgtcgacaaacacttatttcatgacaacgggaaacgctctcggatccgtcaatatgactacgggtgtcgacaatctgttttacattgacaaattccaggtgcgagaagtcaagtaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 100 - DNA, 1982 bp, synthetic construct
  source 1..1982: mol_type = other DNA
ataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgttgcttcaagctttcctttttctgctcgctggtttcgccgctaagattagcgctagcatgacgaacgaaacctctgaccggccgttggtccacttcacccccaacaagggttggatgaacgacccaaacggcctctggtatgatgagaaagacgctaagtggcacctgtacttccagtataaccctaacgatacagtttggggtacgcccttgttttggggccatgcgacctccgacgacctcaccaactgggaagatcagcctatcgcgattgctccgaagcgtaacgattcaggtgcctttagtggcagtatggtcgtagactacaacaacaccagcggtttcttcaacgacactatcgatccacgacagcgctgtgttgccatatggacctacaacacgccggagtccgaggaacaatacattagttactcgctggacgggggatacacctttacagagtaccagaaaaatcccgtcctcgccgcaaactcgactcaattccgcgatcctaaggtattctggtacgagccatcccaaaaatggatcatgacagctgccaagagccaggactataagattgagatctacagcagcgatgacctcaagtcctggaagctcgaaagcgcctttgcgaacgaaggctttcttggctatcagtatgagtgccccggcctcatcgaggtgcctaccgagcaagatcctagcaagagctactgggtgatgttcatctctatcaatccgggagcacccgcgggcggctccttcaaccagtattttgtgggttcgttcaacggaacgcactttgaagcttttgataatcagtcaagagtcgtcgactttggcaaagactactacgccctccagaccttctttaacacggatccaacgtacggttcggcactgggcatcgcgtgggcgtcgaactgggagtatagcgcctttgtccccactaacccttggcgctcgtcgatgtcgttagtgcgtaagttctcgctgaacaccgagtaccaggcaaatccggagacagaactaatcaaccttaaggcagagcctatcttgaacatctctaacgccggaccctggtctaggttcgccaccaacaccacgctcactaaggccaatagttacaatgtcgatctaagcaattcaacgggcacactggaattcgagctcgtttacgcagtcaacaccacccagacgatctcgaaatccgtctttgcagatttgtcgctgtggttcaagggccttgaggacc

SEQ ID NO: 101 - DNA, 500 bp, Moniliella pollinis
  source 1..500: mol_type = other DNA
tatcagatctcagatctcagagagatgtcagagcagacctcgcccttacaaagacacttgtacaaaagttttcaggcatacggattccttcccttcctcgcagaaactctctatcgcccctttgcacttgccgccgtttggtattggacaattgattgctagcaccccttgcaatgatgttatacaaatttatctactgaattgcttgagagtcagtaacaccacggcttccgcgcgagattccatcaccaggcggtggtcacacagatgagttcatcaacgggattctgtcaagtatctcgcatcaacaaatttagctaacaagtctgaacacgttctattcaaattcagaagccacaagaagataactttagtaggaaaagtagattgcaagcacactccgaggcttgctctcgggcgaagaagggatattttgggctcagagcgccatcagaacgacggacgctccaacaaaggggaagcaacagtggtgaacacca

(19) United States
(12) Patent Application Publication
(10) Pub. No.: US 2025/0283028 A1
(43) Pub. Date: Sep. 11, 2025

(54) GENETICALLY MODIFIED YEAST AND FERMENTATION PROCESSES FOR THE PRODUCTION OF XYLITOL

(71) Applicant: Cargill, Incorporated, Wayzata, MN (US)

(72) Inventors: Peter Alan Jauert, Minneapolis, MN (US); Nandita Kohli, Plymouth, MN (US); Christopher Kenneth Miller, Andover, MN (US); Gregory Michael Poynter, Saint Paul, MN (US); Maria Isabel Sardi, Plymouth, MN (US)

(73) Assignee: Cargill, Incorporated, Wayzata, MN (US)

(21) Appl. No.: 18/863,697

(22) PCT Filed: May 5, 2023
(86) PCT No.: PCT/US2023/066627

§ 371 Date: Nov. 7, 2024

Related U.S. Application Data
(60) Provisional application No. 63/364,363, filed on May 9, 2022.

(51) Int. Cl. C12N 1/16; C12N 9/04; C12P 7/18

(52) U.S. Cl. CPC C12N 1/16; C12N 9/0006; C12P 7/18; C12Y 101/01009

(57) ABSTRACT

Disclosed herein are genetically engineered yeast cells capable of producing xylitol. The engineered yeast cell may comprise an exogenous polynucleotide sequence encoding a sugar phosphatase enzyme comprising a sequence at least 60%, at least 65%, at least 70%, at least 75%, at least 80%, at least 85%, at least 90%, at least 95%, at least 98%, at least 99%, or 100% identical to at least one of SEQ ID NOs: 8 and 20.

### CROSS-REFERENCE TO RELATED APPLICATIONS

This application claims the benefit of U.S. Provisional Application No. 63/364,363, filed May 9, 2022, which is incorporated by reference herein in its entirety.

### REFERENCE TO A SEQUENCE LISTING SUBMITTED VIA PATENT CENTER

The content of the Sequence Listing XML file of the sequence listing named “PT-1346-WO-PCT.xml” which is 246,439 bytes in size created on Apr. 13, 2023 and electronically submitted via Patent Center herewith the application is incorporated by reference in its entirety.

### BACKGROUND

Xylitol is a low-calorie sweetener used as a food additive and sugar substitute. Commonly used in drug, dietary supplement, confectionary, and toothpaste compositions, xylitol has also been associated with anticariogenic properties when used in chewing gums. Traditional methods of xylitol production, including chemically catalyzed hydrogenation of xylose hydrolyzed from biomass extracted xylan, are both monetarily and environmentally costly. These methods require high temperatures and pressures, large amounts of water, and metal catalysts that must be mined. In contrast, fermentation processes have been used commercially at large scale to produce other organic molecules, such as ethanol, citric acid, lactic acid, and the like, and may offer a cost effective and sustainable alternative to traditional xylitol processing methods.

Accordingly, provided herein are genetically modified yeast and fermentation methods for the production of xylitol while reducing production of erythritol.

### SUMMARY

The present disclosure provides a genetically engineered yeast cell capable of producing xylitol, the engineered yeast cell comprising an exogenous polynucleotide sequence encoding a xylulokinase (XKS) enzyme comprising a sequence at least 60%, at least 65%, at least 70%, at least 75%, at least 80%, at least 85%, at least 90%, at least 95%, at least 98%, at least 99%, or 100% identical to at least one of SEQ ID NOs:8 and 20. The yeast cell may be an osmotolerant yeast cell. The yeast cell may be a cell of the subphylum Ustilaginomycotina. The yeast cell may be selected from the group consisting of Trichosporonoides megachiliensis, Trychosporonoides oedocephalis, Trychosporonoides nigrescens, Pseudozyma tsukubaensis, Trigonopsis variabilis, Moniliella, Ustilaginomycetes, Trichosporon, Yarrowia lipolytica, Penicillium, Torula, Pichia, Candida, Candida magnoliae, and Aureobasidium. The yeast cell may be a yeast cell of the genus Moniliella.

The disclosure also provides a genetically engineered Moniliella cell capable of producing xylitol, the engineered Monilliela cell comprising an exogenous polynucleotide sequence encoding a xylulokinase (XKS) enzyme comprising a sequence at least 60%, at least 65%, at least 70%, at least 75%, at least 80%, at least 85%, at least 90%, at least 95%, at least 98%, at least 99%, or 100% identical to at least one of SEQ ID NOs:8 and 20.

The XKS enzyme may have a sequence at least 85% identical to at least one of SEQ ID NOs:8 and 20. The XKS enzyme may have a sequence at least 90% identical to at least one of SEQ ID NOs:8 and 20.

The engineered cell described herein may be a Moniliella pollinis cell. The yeast cell described herein may be capable of producing xylitol at a titer of at least 0.5, 1.0, 1.5, 2.0, or 2.5 g/L when used in a fermentation process in the presence of dextrose for 96 hours. Erythritol production by the engineered cell described herein may be reduced relative to erythritol production in an equivalent yeast cell lacking the exogenous polynucleotide sequence.

The exogenous polynucleotide sequence may be operably linked to a heterologous or artificial promoter. The promoter may be a constitutive promoter. The promoter may be selected from the group consisting of pyruvate kinase 1 promoter (PYK1p; SEQ ID NO:61), 6-phosphogluconate dehydrogenase promoter (6PGDp; SEQ ID NO:52), glyceraldehyde-3-phosphate dehydrogenase promoter (TDH3p; SEQ ID NO:54), translational elongation factor 1 promoter (TEFp; SEQ ID NO:55), modified TEFp (SEQ ID NO:53), phosphoglucomutase 1 promoter (PGM1p; SEQ ID NO:56), 3-phosphoglycerate kinase promoter (PGK1p; SEQ ID NO:57), enolase promoter (ENO1p; SEQ ID NO:58), asparagine synthetase promoter (ASNSp; SEQ ID NO:59), 50S ribosomal protein L1 promoter (RPLAp; SEQ ID NO:60), and RPL16B (SEQ ID NO:62).

The exogenous polynucleotide sequence may be integrated into the genome of the yeast cell at a locus selected from the ER1 locus, the ER3 locus, the PDC1 locus, the pyrF locus, the TRP3 locus, the gpdIIA locus, and the gpdIIB locus.

The yeast cell may additionally comprise an exogenous polynucleotide sequence encoding a xylitol dehydrogenase (XDH) enzyme comprising a sequence at least 60%, at least 65%, at least 70%, at least 75%, at least 80%, at least 85%, at least 90%, at least 95%, at least 98%, at least 99%, or 100% identical to at least one of SEQ ID NOs:1, 13, and 14. The XDH enzyme may have a sequence at least 85% identical to at least one of SEQ ID NOs:1, 13, and 14. The XDH enzyme may have a sequence at least 90% identical to at least one of SEQ ID NOs:1, 13, and 14.

The disclosure also provides a method for producing xylitol using the engineered cells described herein, the method comprising contacting a substrate comprising dextrose with an engineered cell described herein, wherein fermentation of the substrate by the engineered yeast produces xylitol. The disclosure also provides a method for producing xylitol, the method comprising contacting a substrate comprising dextrose with an engineered yeast cell comprising an exogenous polynucleotide sequence encoding a sugar phosphatase enzyme comprising a sequence at least 60%, at least 65%, at least 70%, at least 75%, at least 80%, at least 85%, at least 90%, at least 95%, at least 98%, at least 99%, or 100% identical to at least one of SEQ ID NOs:8 and 20, wherein fermentation of the substrate by the engineered yeast produces xylitol. The fermentation temperature may be at or between 25° C. to 45° C., 30° C. to 40° C., or 32° C. to 37° C. The volumetric oxygen uptake rate (OUR) may be between 0.5 to 40, 1 to 35, 2 to 30, 3 to 25, 4 to 20, or 5 to 15 mmol O₂/(L·h). Erythritol production may be reduced relative to an equivalent fermentation run with an equivalent yeast cell lacking the exogenous polynucleotide sequence. Erythritol production may be less than 40, 30, or less than 20 g/L when the fermentation is run at 35° C. for 96 hours. Xylitol production may be at least 0.5, 1.0, 1.5, 2.0, 2.5, or 3.0 g/L when the fermentation is run at 35° C. for 96 hours. Glycerol production may be reduced relative to an equivalent fermentation run with an equivalent yeast cell lacking the exogenous polynucleotide sequence. Ethanol production may be reduced relative to an equivalent fermentation run with an equivalent yeast cell lacking the exogenous polynucleotide sequence.

### BRIEF DESCRIPTION OF THE FIGURES

The drawings illustrate generally, by way of example, but not by way of limitation, various aspects discussed herein.

FIG. 1 shows the native pentose phosphate pathway (dotted lines and arrows) and the native glycolysis pathways (solid lines and arrows) in Moniliella pollinis.

FIG. 2 shows glucose, erythritol, and xylitol metabolite concentrations (g/L) at 96 hours of shake flask fermentations of strains 1-1, 1-2a-c, 1-3a-c, 1-4a-c, 1-5a-c, 1-6a-c, 1-7a-c, 1-8a-c, 1-9a-c, 1-10a-c, 1-11a-c, and 1-12a-c as outlined in Example 2.

FIG. 3 shows xylitol, ribitol, and erythritol metabolite concentrations (g/L) at 96 hours of shake flask fermentations of strains 1-13, 1-14a, 1-14c, 1-8a, 1-8b, and 1-8c both with baffled flasks (BF) and without baffled flasks (no BF), as outlined in Example 3

FIG. 4 shows C6 polyol and xylitol metabolite concentrations (g/L) at 96 hours of shake flask fermentations of strains 1-14c, 1-15a-d, 1-16a-d, 1-17a-d, 1-18a-e, and 1-19a-e, as outlined in Example 4. Data labels report the concentration (g/L) of xylitol.

FIG. 5 shows C6 polyol and xylitol metabolite concentrations (g/L) at 96 hours of shake flask fermentations of strains 1-14c, 1-20a-e, 1-21a-e, 1-23a-e, and 1-24a-e, as outlined in Example 5. Data labels report the concentration (g/L) of xylitol.

FIG. 6 shows ribitol and xylitol metabolite concentrations (g/L) at 96 hours of shake flask fermentations of strains 1-1, 1-20b, 1-24e, 1-25a-d, 1-26a-e, 1-27a-e, and 1-28a-c, as outlined in Example 6. Data labels report the concentration (g/L) of xylitol.

### DETAILED DESCRIPTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

In this document, the terms “a,” “an,” or “the” are used to include one or more than one unless the context clearly dictates otherwise. The term “or” is used to refer to a nonexclusive “or” unless otherwise indicated. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Unless expressly stated, ppm (parts per million), percentage, and ratios are on a by weight basis. Percentage on a by weight basis is also referred to as wt % or % (wt) below.

This disclosure relates to various recombinant cells engineered to produce xylitol. In general, the recombinant cells described herein have an active pentose phosphate pathway and are characterized by expression of an exogenous sugar phosphatase (XKS) enzyme. The recombinant cells described herein may optionally also express an exogenous xylitol dehydrogenase (XDH) enzyme The disclosure further provides fermentation methods for the production of xylitol from dextrose using the genetically engineered cells described herein.

In general, recombinant cells described herein are yeast cells. As used herein, “yeast” refers to eukaryotic single celled microorganisms classified as members of the fungus kingdom. Yeast are unicellular organisms which evolved from multicellular ancestors with some species retaining multicellular characteristics such as forming strings of connected budding cells known as pseudo hyphae or false hyphae. Yeast cells may also be referred to in the art as yeast-like cells, and as used herein “yeast cell” encompasses both yeast and yeast-like cells. Suitable yeast and yeast-like host cells for modification may include, but are not limited to, Saccharomyces cerevisiae, Komagataella sp., Kluyveromyces (e.g., Kluyveromyces lactis, Kluveromyces marxianus), Yarrowia lipolytica, Issatchenkia orientalis, Pichia galeiformis, Pichia sp. YB-4149 (NRRL designation), Pichia pastoris, Candida (e.g., Candida magnoliae, Candida ethanolica), Pichia deserticola, Pichia membranifadens, Pichia fermentans, Aspergillus, Trichoderma, Myceliphthora thermophila, Moniliella (e.g., Moniliella pollinis), Pfaffia, Yamadazyma, Hansenula, Pichia kudriavzevvi, Trichosporonoides (e.g., Trichosporonoides megachiliensis, Trychosporonoides oedocephalis, Trychosporonoides nigrescens), Pseudozyma tsukubaensis, Trigonopsis variabilis, Penicillium, and Torula. An ordinarily skilled artisan would understand the requirements for selection of a suitable yeast cell, and recombinant yeast cells of the present disclosure are not limited to those expressly recited herein. Methods for genetic engineering of yeast cells are known and described in the art and a skilled artisan would understand the methods necessary to transform and engineer a suitable yeast cell.

A suitable yeast cell may be a cell of the phylum Basidiomycota and the subphylum Ustilaginomycotina. Suitable yeast of the subphylum Ustilaginomycotina include, but are not limited to, Ustilago (e.g., U. cynodontis, U. maydis, U. sphaerogena, U. cordal, U. scitaminea, U. coicis, U. syntherismae, U. esculenta, U. neglecta, U. crus-galli, Ustilago avenae), Sporisorium (e.g., Sporisorium exsertum), Moniliella (e.g., M. pollinis, M. tomentosa, M. acetoabutans, M. fonsecae, M. madida, M. megachiliensis, M. ocedocephalis, M. nigrescens), and Pseudozyma (e.g., Pseudozyma tsukubaensis), and Trichosporonoides (e.g., Trichosporonoides megachiliensis, Trychosporonoides oedocephalis, Trychosporonoides nigrescens). Yeast of the subphylum Ustilaginomycotina have been known and described in the art as potential production organisms for valuable chemicals such as itaconate, malate, succinate, mannitol, and erythritol and other valuable biotechnological applications. See, for example, Geiser et al. (Prospecting the biodiversity of the fungal family Ustilaginacceae for the production of value-added chemicals,” Fungal Biol Biotechnol, 2014, 1:2), Feldbrugge et al., (“The biotechnological use and potential of plant pathogenic smut fungi,” Appl Microbiol Biotechnol, 2013, 97(8):3253-65), Guevarra et al., (“Accumulation of itaconic, 2-hydroxyparaconic, itatartaric, and malic acids by strains of the genus Ustilago, Agric. Biol. Chem., 1990, 54(9), 2353-2358), and Moon et al., (“Biotechnological production of erythritol and its applications,” Appl Microbiol Biotechnol, 2010, 86:1017-1025).

A suitable yeast cell will have an active pentose phosphate pathway that produces ribulose-5-phosphate. As used herein “active pentose phosphate pathway” refers to expression of one or more functional enzymes which, together, convert glucose-6-phosphate, NADP⁺ or NAD+, and water to NADPH or NADH, C02, and ribulose-5-phosphate. Continuing in a non-oxidative phase, the pathway may also produce other pentose (i.e., 5-carbon) sugars. For example, the pentose phosphate pathway may produce ribulose-5-phosphate, ribose-5-phosphate, xylulose-5-phosphate, fructose 6-phosphate, combinations thereof, and the like, depending on the enzymatic activities present. The active pentose phosphate pathway may be native to the yeast cell, or it may be introduced into the yeast cell by genetic engineering.

The yeast cell may be an osmotolerant yeast cell. As used herein, “osmotolerant” refers to a yeast capable of growth and reproduction under conditions of high osmolarity, such as at least 10% (w/v), at least 20% (w/v), at least 30% (w/v), at least 40% (w/v), at least 50% (w/v), or at least 60% (w/v) glucose and/or at least 6% (w/v), at least 10% (w/v), at least 12% (w/v), at least 13% (w/v), at least 15% (w/v) sodium chloride. Species and strains of osmotolerant yeast are known and described in the art, including many species of yeast used in industrial fermentation processes. Likewise, methods for assaying yeast osmotolerance are known and described in the art. See, for example, Tiwari, S., et al., (“Nectar yeast community of tropical flowering plants and assessment of their osmotolerance and xylitol-producing potential,” Current Microbiology, 2022, 79:28).

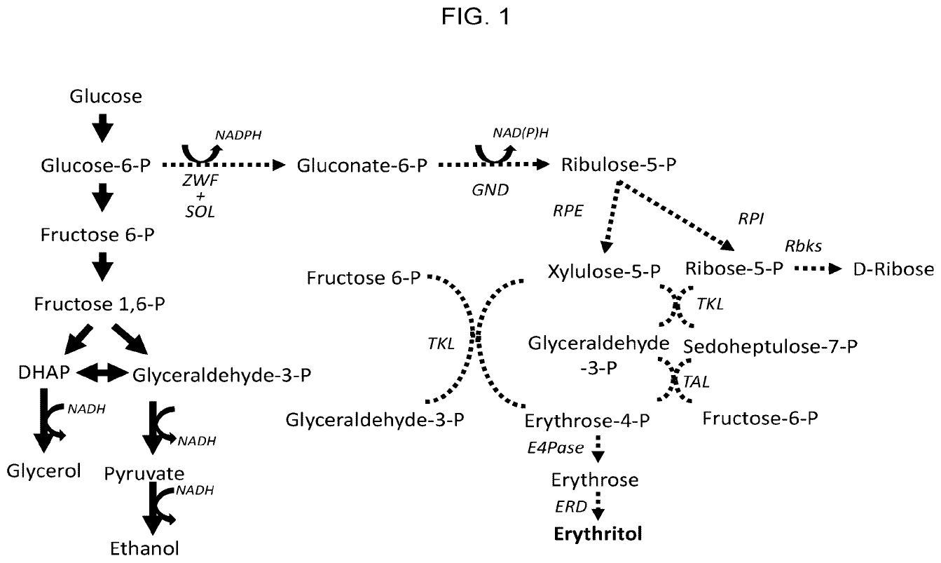

The recombinant yeast cell may be a recombinant Moniliella cell, for example, a Moniliella pollinis cell. FIG. 1 shows the predicted native pentose phosphate and glycolysis pathways in Moniliella pollinis. Moniliella has previously been used in the fermentation production of erythritol and methods for genetically modifying and fermenting Moniliella are known and described in the art. See, for example, Li et al. (“Methods for genetic transformation of filamentous fungi,” 2017, Microb Cell Fact, 16:168).

Various plasmids and methods for transformation of Moniliella are also described in the Examples below. For example, Moniliella may be transformed using a bipartite polynucleotide sequence in which, following recombination, the exogenous polynucleotide of interest is integrated at the specified locus and the selection marker is expressible within the cell. Suitable selection markers are known and used in the art. The selectable marker may include, but is not limited to, amdS (for example broken into a 3′ portion, SEQ ID NO:91, and a 5′ portion, SEQ ID NO:92), G418 resistance gene (for example broken into a 3′ portion, SEQ ID NO:97, and a 5′ portion, SEQ ID NO:98), zeocin resistance gene (for example broken into a 3′ portion, SEQ ID NO:93, and a 5′ portion, SEQ ID NO:94), nourseothricin N-acetyl transferase (NAT) (for example broken into a 3′ portion, SEQ ID NO:95, and a 5′ portion, SEQ ID NO:96), and invertase gene (SUC2) (for example a 3′ portion of SEQ ID NO:99 and a 5′ portion of SEQ ID NO:100).

The recombinant cells described herein include one or more exogenous polynucleotide sequences encoding one or more exogenous polypeptides that, when expressed improve the fermentation of glucose to ribitol by the recombinant cells.

The terms “glucose” and “dextrose” are used interchangeably herein and refer to D-glucose except where expressly indicated otherwise.

As used herein, “exogenous” refers to genetic material or an expression product thereof that originates from outside of the host organism. For example, the exogenous genetic material or expression product thereof can be a modified form of genetic material native to the host organism, it can be derived from another organism, it can be a modified form of a component derived from another organism, or it can be a synthetically derived component. For example, a K. lactis invertase gene is exogenous when introduced into S. cerevisiae.

As used herein, “native” refers to genetic material or an expression product thereof that is found, apart from individual-to-individual mutations which do not affect function or expression, within the genome of wild-type cells of the host cell. For the purposes of this application, the Moniliella pollinis cell “Moniliella tomentosa var pollinis TCV364” described in U.S. Pat. No. 6,440,712, which is incorporated herein by reference in its entirety, and deposited under the Budapest Treaty at BCCM/MUCL (Belgian Coordinated Collections of Micro-organisms/Mycothèque de l'Université Catholique de Louvain by Eridania Béghin Say, Vilvoorde R&D Centre, Havenstraat 84, B-1800 Vilvoorde) on Mar. 28, 1997 under number MUCL40385, is considered the wild-type Moniliella pollinis cell.

As used herein, the terms “polypeptide” and “peptide” are used interchangeably and refer to the collective primary, secondary, tertiary, and quaternary amino acid sequences and structure necessary to give the recited macromolecule its function and properties. As used herein, “enzyme” or “biosynthetic pathway enzyme” refer to a protein that catalyzes a chemical reaction. The recitation of any particular enzyme, either independently or as part of a biosynthetic pathway is understood to include the co-factors, co-enzymes, and metals necessary for the enzyme to properly function. A summary of the amino acids and their three and one letter symbols as understood in the art is presented in Table 1. The amino acid name, three letter symbol, and one letter symbol are used interchangeably herein.

TABLE 1
Amino Acid three and one letter symbols

| | Three letter | One letter |
|---|---|---|
| Amino Acid | symbol | symbol |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Variants or sequences having substantial identity or homology with the polypeptides described herein can be utilized in the practice of the disclosed recombinant cells, compositions, and methods. Such sequences can be referred to as variants or modified sequences. That is, a polypeptide sequence can be modified yet still retain the ability to exhibit the desired activity. Generally, the variant or modified sequence may include greater than about 45%, 45%, 50%, 55%, 60%, 65%, 70%, 75%, 80%, 85%, 90%, or 95% sequence identity with the wild type, naturally occurring polypeptide sequence, or with a variant polypeptide as described herein.

As used herein, the phrases “% sequence identity,” “% identity,” and “percent identity,” are used interchangeably and refer to the percentage of residue matches between at least two amino acid sequences or at least two nucleic acid sequences aligned using a standardized algorithm. Methods of amino acid and nucleic acid sequence alignment are well-known. Sequence alignment and generation of sequence identity include global alignments and local alignments which are carried out using computational approaches. An alignment can be performed using BLAST (National Center for Biological Information (NCBI) Basic Local Alignment Search Tool) version 2.2.31 software with default parameters. Amino acid % sequence identity between amino acid sequences can be determined using standard protein BLAST with the following default parameters: Max target sequences: 100; Short queries: Automatically adjust parameters for short input sequences; Expect threshold: 10; Word size: 6; Max matches in a query range: 0; Matrix: BLOSUM62; Gap Costs: (Existence: 11, Extension: 1); Compositional adjustments: Conditional compositional score matrix adjustment; Filter: none selected; Mask: none selected. Nucleic acid % sequence identity between nucleic acid sequences can be determined using standard nucleotide BLAST with the following default parameters: Max target sequences: 100; Short queries: Automatically adjust parameters for short input sequences; Expect threshold: 10; Word size: 28; Max matches in a query range: 0; Match/Mismatch Scores: 1, −2; Gap costs: Linear; Filter: Low complexity regions; Mask: Mask for lookup table only. A sequence having an identity score of XX % (for example, 80%) with regard to a reference sequence using the NCBI BLAST version 2.2.31 algorithm with default parameters is considered to be at least XX % identical or, equivalently, have XX % sequence identity to the reference sequence.

Polypeptide or polynucleotide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The polypeptides disclosed herein may include “variant” polypeptides, “mutants,” and “derivatives thereof.” As used herein the term “wild-type” is a term of the art understood by skilled persons and means the typical form of a polypeptide as it occurs in nature as distinguished from variant or mutant forms. As used herein, a “variant,” “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule.

The amino acid sequences of the polypeptide variants, mutants, derivatives, or fragments as contemplated herein may include conservative amino acid substitutions relative to a reference amino acid sequence. For example, a variant, mutant, derivative, or fragment polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge and/or hydrophobicity of the molecule at the site of the substitution, and/or (c) the bulk of the side chain.

As used herein, terms “polynucleotide,” “polynucleotide sequence,” and “nucleic acid sequence,” and “nucleic acid,” are used interchangeably and refer to a sequence of nucleotides or any fragment thereof. These phrases also refer to DNA or RNA of natural or synthetic origin, which may be single-stranded or double-stranded and may represent the sense or the antisense strand. The DNA polynucleotides may be a cDNA (e.g., coding DNA) or a genomic DNA sequence (e.g., including both introns and exons).

A polynucleotide is said to encode a polypeptide if, in its native state or when manipulated by methods known to those skilled in the art, it can be transcribed and/or translated to produce the polypeptide or a fragment thereof. The anti-sense strand of such a polynucleotide is also said to encode the sequence.

Those of skill in the art understand the degeneracy of the genetic code and that a variety of polynucleotides can encode the same polypeptide. In some aspects, the polynucleotides (e.g., polynucleotides encoding an XKS or XDH polypeptide) may be codon-optimized for expression in a particular cell including, without limitation, a plant cell, bacterial cell, fungal cell, or animal cell. While polypeptides encoded by polynucleotide sequences found in various species are disclosed herein any polynucleotide sequences may be used which encodes a desired form of the polypeptides described herein. Thus, non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

The recombinant cells described herein may include deletions or disruptions in one or more native genes. The phase “deletion or disruption” refers to the status of a native gene in the recombinant cell that has either a completely eliminated coding region (deletion) or a modification of the gene, its promoter, or its terminator (such as by a deletion, insertion, or mutation) so that the gene no longer produces an active expression product, produces severely reduced quantities of the expression product (e.g., at least a 75% reduction or at least a 90% reduction) or produces an expression product with severely reduced activity (e.g., at least 75% reduced or at least 90% reduced). The deletion or disruption can be achieved by genetic engineering methods, forced evolution, mutagenesis, RNA interference (RNAi), and/or selection and screening. The native gene to be deleted or disrupted may be replaced with an exogenous nucleic acid of interest for the expression of an exogenous gene product (e.g., polypeptide, enzyme, and the like).

The recombinant cells described herein may include one or more genetic modifications in which an exogenous nucleic acid is integrated into the genome of the host cell. One of skill in the art know how to select suitable loci in a yeast genome for integration of the exogenous nucleic acid. Suitable integration loci may include, but are not limited to, the PDC1, GPD1, CYB2A, CYB2B, g4240, YMR226, MDHB, ATO2, Adh9091, Adh1202, ADE2, ADH2556, GAL6, MDH1, SCW11, ER1, ER3, pyrF, TRP3, gpdIIA, and gpdIIB loci. For example, in a M. pollinis host cells, suitable interaction loci may include, but are not limited to, the ER1 locus (defined as the locus flanked by SEQ ID NO:87 and SEQ ID NO:88), the ER3 locus (defined as the locus flanked by SEQ ID NO:81 and SEQ ID NO:82), the PDC1 locus (defined as the locus flanked by SEQ ID NO:75 and SEQ ID NO:76), the pyrF locus (defined as the locus flanked by SEQ ID NO:77 and SEQ ID NO:78), the TRP3 locus (defined as the locus flanked by SEQ ID NO:83 and SEQ ID NO:84), the gpdIIA locus (defined as the locus flanked by SEQ ID NO:85 and SEQ ID NO:86); and the gpdIIB locus (defined as the locus flanked by SEQ ID NO:89 and SEQ ID NO:90). The exogenous nucleic acid may also be integrated in an intergenic region or other location in the host cell genome not specifically specified herein. Other suitable integration loci may be determined by one of skill in the art. Furthermore, one of skill in the art would recognize how to use sequences to design primers to verify correct gene integration at the chosen locus.

The recombinant cell may have one or more copies of a given exogenous nucleic acid sequence integrated in a host chromosome(s) and replicated together with the chromosome(s) into which it has been integrated. For example, the yeast cell may be transformed with nucleic acid construct including a polynucleotide sequence encoding for a polypeptide described herein and the polynucleotide sequence encoding for the polypeptide may be integrated in one or more copies in a host chromosome(s). The recombinant cell may include multiple copies (two or more) of a given polynucleotide sequence encoding a polypeptide described herein. The recombinant cell may have one, two, three, four, five, six, seven, eight, nine, ten, or more copies of a polynucleotide sequence encoding a polypeptide described herein integrated into the genome. The multiple copies of said polynucleotide sequence may all be incorporated at a single locus or may be incorporated at multiple loci.

The recombinant cells described herein are capable of producing xylitol and include an exogenous polynucleotide sequence encoding a xylulokinase (XKS) enzyme. The exogenous polynucleotide sequence may be an exogenous xylulose sugar phosphatase (XKS) gene.

A “xylulokinase gene” and an “XKS gene” are used interchangeably herein and refer to any gene or polynucleotide that encodes a polypeptide with xylulokinase activity. As used herein, “xylulokinase activity” refer to the ability to catalyze the conversion of xylulose-5-phosphate and ADP to xylulose and ATP. The XKS gene may be derived from any suitable source. For example, the XKS gene may be derived from Saccharomyces cerevisiae. The XKS gene may encode an amino acid at least 50%, at least 60%, at least 70%, at least 80%, at least 85%, at least 90%, at least 95%, at least 97%, at least 99%, or 100% sequence identity to the amino acid sequence of at least one of SEQ ID NOs:8 and 20.

The recombinant cell may comprise an exogenous polynucleotide that is or may be derived from a Saccharomyces cerevisiae DOG1 sugar phosphatase gene encoding the amino acid of SEQ ID NO:8. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:8.

The recombinant cell may comprise an exogenous polynucleotide that is or may be derived from a Saccharomyces cerevisiae DOG2 sugar phosphatase gene encoding the amino acid of SEQ ID NO:20. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:20.

Recombinant cells described herein may additionally include an exogenous polynucleotide sequence encoding a xylitol dehydrogenase (XDH) enzyme. The exogenous polynucleotide sequence may be an exogenous xylitol dehydrogenase (XDH) gene.

A “xylitol dehydrogenase gene” and an “XDH gene” are used interchangeably herein and refer to any gene or polynucleotide that encodes a polypeptide with xylitol dehydrogenase activity. As used herein, “xylitol dehydrogenase activity” refer to the ability to catalyze the conversion of xylulose and NADH or NADPH to xylitol and NAD⁺ or NADP⁺. The XDH gene may be derived from any suitable source. For example, the XDH gene may be derived from Pichia stipitis, Rhodobacteraceae bacterium, or Bemisia argentofolii. The XDH gene may encode an amino acid at least 50%, at least 60%, at least 70%, at least 80%, at least 85%, at least 90%, at least 95%, at least 97%, at least 99%, or 100% sequence identity to the amino acid sequence of at least one of SEQ ID NOs:1, 13, or 14.

The recombinant cell may comprise an exogenous polynucleotide that is, or may be derived from, a cofactor switched Pichia stipitis XDH gene encoding the amino acid of SEQ ID NO:1. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:1.

The recombinant cell may comprise an exogenous polynucleotide that is, or may be derived from, a Rhodobacteraceae bacterium SDR family oxidoreductase gene encoding the amino acid of SEQ ID NO:13. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:13.

The recombinant cell may comprise an exogenous polynucleotide that is, or may be derived from, a Bemisia argentofolii (Silverleaf Whitefly) ketose reductase (sorbitol dehydrogenase) gene encoding the amino acid of SEQ ID NO:14. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:14.

The exogenous polynucleotides in the recombinant cells described herein may be under the control of a promoter. For example, the exogenous nucleic acid may be operably linked to a heterologous or artificial promoter. Suitable promoters are known and described in the art. Promoters may include, but are not limited to, pyruvate decarboxylase promoter (PDC), translation elongation factor 2 promoter (TEF2), SED1, alcohol dehydrogenase 1A promoter (ADH1), hexokinase 2 promoter (HXK2), FLO5 promoter, pyruvate kinase 1 promoter (PYK1p; SEQ ID NO:61); 6-phosphogluconate dehydrogenase promoter (6PGDp; SEQ ID NO:52); glyceraldehyde-3-phosphate dehydrogenase promoter (TDH3p; SEQ ID NO:54); translational elongation factor 1 promoter (TEFp; SEQ ID NO:55); modified TEFp (SEQ ID NO:53); phosphoglucomutase 1 promoter (PGM1p; SEQ ID NO:56); 3-phosphoglycerate kinase promoter (PGK1p; SEQ ID NO:57); enolase promoter (ENO1p; SEQ ID NO:58); asparagine synthetase promoter (ASNSp; SEQ ID NO:59); 50S ribosomal protein L1 promoter (RPLAp; SEQ ID NO:61); and RPL16B (SEQ ID NO:62).

The exogenous nucleic acids in the recombinant cells described herein may be under the control of a terminator. For example, the exogenous nucleic acid may be operably linked to a heterologous or artificial terminator. Suitable terminators are known and described in the art. Terminators may include, but are not limited to, GAL10 terminator, PDC terminator, transaldolase terminator (TAL), 6PGD terminator (6PGDt; SEQ ID NO:63); ASNS terminator (ASNSt; SEQ ID NO:64); ENO1 terminator (ENO1t; SEQ ID NO:65); hexokinase 1 terminator (HXK1t; SEQ ID NO:66); PGK1 terminator (PGK1t; SEQ ID NO:67); PGM1 terminator (PGM1t; SEQ ID NO:68); PYK1 terminator (PYK1t; SEQ ID NO:69); RPLA terminator (RPLAt: SEQ ID NO:70); transaldolase 1 terminator (TAL1t; SEQ ID NO:71); TDH3 terminator (TDH3t; SEQ ID NO:72); translation elongation factor 2 terminator (TEF2t; SEQ ID NO:73); triosephosphate isomerase 1 terminator (TPI1t; SEQ ID NO:74); and MpTEF1t (SEQ ID NO:101).

A promoter or terminator is “operably linked” to a given polynucleotide (e.g., a gene) if its position in the genome or expression cassette relative to said polynucleotide is such that the promoter or terminator, as the case may be, performs its transcriptional control function.

The polypeptides described herein may be provided as part of a construct. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies. The construct may be a vector including a promoter operably linked to the polynucleotide encoding a polypeptide as described herein. As used herein, the term “vector” refers to a polynucleotide capable of transporting another polynucleotide to which it has been linked. The vector may be a plasmid, which refers to a circular double-stranded DNA loop into which additional DNA segments may be integrated.

The disclosure also provides fermentation methods for the production of xylitol using the recombinant cells described herein. The fermentation methods include the step of fermenting a substrate using the genetically engineered yeasts described herein to product xylitol. The fermentation method can include additional steps, as would be understood by a person skilled in the art. Non-limiting examples of additional process steps include maintaining the temperature of the fermentation broth within a predetermined range, adjusting the pH during fermentation, and isolating the xylitol from the fermentation broth. The fermentation process may be a fully aerobic process or a partially aerobic process.

The fermentation method can be run using a suitable fermentation substrate. The substrate of the fermentation method can include glucose, sucrose, galactose, mannose, molasses, xylose, fructose, hydrolysates of starch, lignocellulosic hydrolysates, or a combination thereof. One skilled in the art will recognize what fermentation substrate is suitable for a given fermentation organism and system.

The fermentation process can be run under various conditions. The fermentation temperature, i.e., the temperature of the fermentation broth during processing, may be ambient temperature. Alternatively, or additionally, the fermentation temperature may be maintained within a predetermined range. For example, the fermentation temperature can be maintained in the range of 25° C. to 45° C., 30° C. to 40° C., or 32° C. to 37° C., preferably about 35° C. However, a skilled artisan will recognize that the fermentation temperature is not limited to any specific range or temperature recited herein and may be modified as appropriate.

The fermentation process can be run within certain oxygen uptake rate (OUR) ranges. The volumetric OUR of the fermentation process can be in the range of 0.5 to 40, 1 to 35, 2 to 30, 3 to 25, 4 to 20, or 5 to 15 mmol O₂/(L·h). In some embodiments, the specific OUR can be in the range of 0.05 to 10, 0.1 to 8, 0.15 to 5, 0.2 to 1, or 0.3 to 0.75 mmol O₂/(g cell dry weight·h). However, the volumetric or specific OURs of the fermentation process are not limited to any specific rates or ranges recited herein.

The fermentation process can be run at various cell concentrations. In some embodiments, the cell dry weight at the end of fermentation can be 5 to 40, 8 to 30, or 10 to 20 g cell dry weight/L. Further, the pitch density or pitching rate of the fermentation process can vary. In some embodiments, the pitch density can be 0.05 to 11, 0.1 to 10, or 0.25 to 8 g cell dry weight/L.

The initial dextrose concentration of the fermentation may be at least 100, 200, 250, 300, 350, or at least 400 g/L dextrose. The initial dextrose concentration may be between 100 to 400, 150 to 350, or 250 to 325 g/L.

The fermentation process can be associated with various characteristics, such as, but not limited to, fermentation production rate, pathway fermentation yield, final titer, and peak fermentation rate. These characteristics can be affected by the selection of the yeast and/or genetic modification of the yeast used in the fermentation process. These characteristics can be affected by adjusting the fermentation process conditions. These characteristics can be adjusted via a combination of yeast selection or modification and the selection of fermentation process conditions.

The xylitol production rate of the process may be at least 0.2, 0.3, 0.5, 0.75, or at least 1.0 g L⁻¹ h⁻¹. The final xylitol titer of the process may be at least 0.5, 1.0, 1.5, 2.0, 2.5 or 3.0 g/L.

The fermentation process can be run as a dextrose-fed batch. Further, the fermentation process can be a batch process, continuous process, or semi-continuous process, as would be understood by a person skilled in the art.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

### Example 1—Genetically Modified Moniliella pollinis Strains

Strain 1-1 is the Moniliella pollinis host strain “Moniliella tomentosa var pollinis TCV364” described in U.S. Pat. No. 6,440,712, which is incorporated herein by reference in its entirety, and deposited under the Budapest Treaty at BCCM/MUCL (Belgian Coordinated Collections of Micro-organisms/Mycothèque de l'Université Catholique de Louvain by Eridania Béghin Say, Vilvoorde R&D Centre, Havenstraat 84, B-1800 Vilvoorde) on Mar. 28, 1997, under number MUCL40385. Table 2 below lists various Moniliella pollinis strains, including information on the parent strain, the sequence with which the parent strain was transformed, and characterizations of the expression cassette(s) contained on the transformed sequence.

In Table 2, the following cassettes were utilized. Each “XHD Homolog+3′ G418 Expression Cassette” contained, in order, a 5′ ER1 flanking sequence (SEQ ID NO:87), a MpPGM1 promoter (SEQ ID NO:56), a gene encoding the indicated XDH enzyme homolog (one of SEQ ID NOs:1, 13, or 14), a MpPGK1 terminator (SEQ ID NO:67), and a 3′ portion of a G418 resistance gene (SEQ ID NO:97). Each “XKS/Phosphatase Homolog+5′ G418 Expression Cassette” contained, in order, a 5′ portion of a G418 resistance gene (SEQ ID NO:98), a MpPGK promoter (SEQ ID NO:57), a gene encoding the indicated XKS/Phosphatase enzyme homolog (one of SEQ ID NOs:35 or 41-50), a MpENO1 terminator (SEQ ID NO:65), and a 3′ ER1 flanking sequence (SEQ ID NO:88).

In Table 3, the following cassettes were utilized. Each “XDH Homolog Expression Cassette” contained, in order, a 5′ ER1 flanking sequence (SEQ ID NO:87), a MpPYK1 promoter (SEQ ID NO:61), a gene encoding the indicated XDH homolog (one of SEQ ID NOs:15-19), a Mp6PGD terminator (SEQ ID NO:63), and a 5′ portion of a Zeocin resistance gene (SEQ ID NO:93). The “Selection Marker Cassette” of SEQ ID NO:38 contained, in order, a 3′ portion of a Zeocin selection marker gene (SEQ ID NO:93), an MpTEF2 terminator (SEQ ID NO:73), and a 3′ ER1 flanking sequence (SEQ ID NO:88). Each “XKS/Phosphatase Expression Cassette” contained, in order, a 5′ ER1 flanking sequence (SEQ ID NO:87), a PGK1 promoter (SEQ ID NO:67), a gene encoding the indicated XKS/Phosphatase enzyme homolog (one of SEQ ID NOs:8 or 20-24), MpENO1 terminator (SEQ ID NO:65), a 3′ portion of either a G418 (SEQ ID NO:97) or a Zeocin (SEQ ID NO:93) resistance gene, and an MpTEF2 terminator (SEQ ID NO:73). The “Selection Marker Cassette” of SEQ ID NO:37 contained, in order, a 5′ portion of a Zeocin resistance gene and a 3′ ER1 flanking sequence (SEQ ID NO:88). The “Selection Marker Cassette” of SEQ ID NO:36 contained, in order, a 5′ portion of a G418 resistance gene and a 3′ ER1 flanking sequence (SEQ ID NO:88).

Upon bipartite transformation with both cassettes as indicated in Tables A and B, the two cassettes recombine for integration of both the nucleotide sequence encoding the indicated XDH or XKS/phosphatase homolog and the indicated selectable marker at the ER1 loci.

The indicated Moniliella pollinis parent strain was transformed with the indicated sequence(s) by first protoplasting the parent strain by adding an enzyme mixture containing 0.6M MgSO₄, 7.5 g/L driselase, and 12.5 g/L Trichoderma harzianum lysing enzyme to a mycelial pellet of the parent strain. Protoplasts were then pelleted, washed with 0.6M MgSO₄, and resuspended in STC medium (0.6M sucrose, 50 mM CaCl2), 10 mM Tris-HCl, pH 7.5). Bipartite transformations were prepared by adding 100 μg single stranded salmon sperm DNA and 1.5 to 5 μg each of the 5′ and 3′ DNA transformation fragments (3-10 μg total; see Table 4 for list of fragments) to approximately 200 μL protoplast mixture (10⁸ cells/mL). 1 mL 50% PEG in STC medium was then added to the salmon sperm DNA, transformation DNA, and protoplast mixture and the resulting combination was incubated for 15 minutes at room temperature. Following incubation, recovery broth (0.4M sucrose, 1 g/L yeast extract, 1 g/L malt extract, 10 g/L glucose, pH 4.5) was added to the mixture and incubated at 27° C., 100 rpm, for 16 to 24 hours. Following the incubation, protoplasts were pelleted by centrifugation and resuspended in 1 mL PBS.

The resuspended protoplasts were plated on PDA+250 mg/L geneticin (G418) or PDA+100 mg/L zeocin selection plates and incubated at 30-35° C. for at least 2-4 days until transformants grow. Resulting transformants were evaluated by colony PCR for integration of the indicated sequence. A PCR verified isolate was then designated as the indicated strain number. In some instances, more than one PCR verified isolate, e.g., “sister” isolates, are indicated by letters following the strain number. For example, strain 1-2 has 3 sister isolates, strains 1-2a, 1-2b, and 1-2c.

For example, Strain 1-1 was transformed with SEQ ID NO:39 and SEQ ID NO:41. SEQ ID NO:39 contains i) a 5′ ER1 flanking sequence (SEQ ID NO:87), ii) a MpPGM1 promoter (SEQ ID NO:56), iii) an expression cassette for the cofactor switched XDH from Pichia stipitis of SEQ ID NO:1, iv) a MpPGK1 terminator (SEQ ID NO:67), and v) a 3′ portion of a G418 resistance gene (SEQ ID NO:97). SEQ ID NO:41 contains i) a 5′ portion of a G418 resistance gene (SEQ ID NO:98), ii) a MpPGK promoter (SEQ ID NO:57), iii) an expression cassette encoding the Hypsizygus marmoreus XKS1 of SEQ ID NO:2, iv) a MpENO1 terminator (SEQ ID NO:65), and v) a 3′ ER1 flanking sequence (SEQ ID NO:88). Transformants were selected on PDA+250 mg/L geneticin (G418) selection plates and incubated at 35° C. for at least 2 days until transformants grow. Resulting transformants were streaked for single colony isolation on PDA+geneticin (G418) plates and single colonies were selected. Selected colonies were evaluated by colony PCR for integration of the indicated sequence. PCR verified isolates were designated strains 1-2a, 1-2b, and 1-2c.

TABLE 2

| | | XDH Homolog + 3′ G418 | | | XKS/Phosphatase Homolog + 5′ | | |
|---|---|---|---|---|---|---|---|
| | | Expression Cassette | | | G418 Expression Cassette | | |
| | | | Polypeptide Source | Encoded | | Polypeptide Source | Encoded |
| | | | Organism (Gene | Polypeptide | | Organism (Gene | Polypeptide |
| | Parent | Transformation | Name and/or | SEQ ID | Transformation | Name and/or | SEQ ID |
| Strain | Strain | SEQ ID NO: | Accession Number) | NO: | SEQ ID NO: | Accession Number) | NO: |
| 1-2a-c | 1-1 | 39 | Pichia stipitis cofactor | 1 | 41 | Hypsizygus marmoreus | 2 |
| | | | switched XDH | | | XKS1 | |
| 1-3a-c | 1-1 | 39 | Pichia stipitis cofactor | 1 | 42 | Cylindrobasidium torrendii | 3 |
| | | | switched XDH | | | XKS1 | |
| 1-4a-c | 1-1 | 39 | Pichia stipitis cofactor | 1 | 43 | Pleurotus ostreatus XKS1 | 4 |
| | | | switched XDH | | | | |
| 1-5a-c | 1-1 | 39 | Pichia stipitis cofactor | 1 | 44 | Trametes coccinea XKS1 | 5 |
| | | | switched XDH | | | | |
| 1-6a-c | 1-1 | 39 | Pichia stipitis cofactor | 1 | 45 | Antrodiella citronella | 6 |
| | | | switched XDH | | | XKS1 | |
| 1-7a-c | 1-1 | 39 | Pichia stipitis cofactor | 1 | 46 | Ustilago maydis XKS1 | 7 |
| | | | switched XDH | | | | |
| 1-8a-c | 1-1 | 39 | Pichia stipitis cofactor | 1 | 35 | Saccharomyces cerevisiae | 8 |
| | | | switched XDH | | | DOG1 | |
| 1-9a-c | 1-1 | 39 | Pichia stipitis cofactor | 1 | 47 | Bacteroides | 9 |
| | | | switched XDH | | | thetaiotaomicron XKS1 | |
| 1-10a-c | 1-1 | 39 | Pichia stipitis cofactor | 1 | 48 | Pichia stipitis XKS | 10 |
| | | | switched XDH | | | | |
| 1-11a-c | 1-1 | 39 | Pichia stipitis cofactor | 1 | 49 | Saccharomyces cerevisiae | 11 |
| | | | switched XDH | | | XKS | |
| 1-12a-c | 1-1 | 39 | Pichia stipitis cofactor | 1 | 50 | Streptomyces avermitilis | 12 |
| | | | switched XDH | | | HAD | |
| 1-13 | 1-1 | 40 | Rhodobacteraceae bacterium | 13 | 35 | Saccharomyces cerevisiae | 8 |
| | | | SDR family oxidoreductase | | | DOG1 | |
| 1-14a-c | 1-1 | 51 | Bemisia argentofolii | 14 | 35 | Saccharomyces cerevisiae | 8 |
| | | | (Silverleaf Whitefly) ketose | | | DOG1 | |
| | | | reductase (sorbitol | | | | |
| | | | dehydrogenase) | | | | |

TABLE 3

| | | XDH Homolog Expression | | | XKS/Phosphatase Expression | | | | |
|---|---|---|---|---|---|---|---|---|---|
| | | Cassette | | | Cassette | | | | |
| | | | Polypeptide | | | Polypeptide | | | |
| | | | Source Organism | Encoded | | Source Organism | Encoded | Selectable Marker | |
| | | | (Gene Name | Polypeptide | | (Gene Name | Polypeptide | Cassette | |
| | Parent | Transformation | and/or Accession | SEQ ID | Transformation | and/or Accession | SEQ ID | Selectable | Transformation |
| Strain | Strain | SEQ ID NO: | Number) | NO: | SEQ ID NO: | Number) | NO: | Marker | SEQ ID NO: |
| 1-15a-d | 1-14c | 25 | Bos indicus × | 15 | — | — | — | Zeocin | 38 |
| | | | Bos taurus XDH | | | | | | |
| | | | (XP_027409920) | | | | | | |
| 1-16a-d | 1-14c | 26 | Aspergillus | 16 | — | — | — | Zeocin | 38 |
| | | | tubingensis XDH | | | | | | |
| | | | (GFN13953) | | | | | | |
| 1-17a-e | 1-14c | 27 | Chaetomium | 17 | — | — | — | Zeocin | 38 |
| | | | thermophilum | | | | | | |
| | | | var. | | | | | | |
| | | | thermophilum | | | | | | |
| | | | DSM 1495 XDH | | | | | | |
| | | | (EGS17058) | | | | | | |
| 1-18a-e | 1-14c | 28 | Candidatus | 18 | — | — | — | Zeocin | 38 |
| | | | Poribacteria | | | | | | |
| | | | bacterium XDH | | | | | | |
| | | | (RKY04443) | | | | | | |
| 1-19a-e | 1-14c | 29 | Linepithema | 19 | — | — | — | Zeocin | 38 |
| | | | humile XDH | | | | | | |
| | | | (XP_012217409) | | | | | | |
| 1-20a-e | 1-14c | — | — | — | 30 | Saccharomyces | 20 | Zeocin | 37 |
| | | | | | | cerevisiae | | | |
| | | | | | | (KZV10766) | | | |
| | | | | | | Sugar | | | |
| | | | | | | Phosphatase | | | |
| | | | | | | DOG2 | | | |
| 1-21a-e | 1-14c | — | — | — | 31 | Acidobacteria | 21 | Zeocin | 37 |
| | | | | | | bacterium Sugar | | | |
| | | | | | | Phosphatase | | | |
| | | | | | | (PYX10474) | | | |
| 1-22 | 1-14c | — | — | — | 32 | Mesorhizobium | 22 | Zeocin | 37 |
| | | | | | | sp. Sugar | | | |
| | | | | | | Phosphatase | | | |
| | | | | | | (TIO13298) | | | |
| 1-23a-e | 1-14c | — | — | — | 33 | Escherichia coli | 23 | Zeocin | 37 |
| | | | | | | Sugar | | | |
| | | | | | | Phosphatase | | | |
| | | | | | | (EFE9125297) | | | |
| 1-24a-e | 1-14c | — | — | — | 34 | Puerhibacterium | 24 | Zeocin | 37 |
| | | | | | | puerhi Sugar | | | |
| | | | | | | Phosphatase | | | |
| | | | | | | (WP_159795540) | | | |
| 1-25a-d | 1-1 | — | — | — | 35 | Saccharomyces | 8 | G418 | 36 |
| | | | | | | cerevisiae DOG1 | | | |
| 1-26a-e | 1-1 | — | — | — | 30 | Saccharomyces | 20 | Zeocin | 37 |
| | | | | | | cerevisiae | | | |
| | | | | | | (KZV10766) | | | |
| | | | | | | Sugar | | | |
| | | | | | | Phosphatase | | | |
| | | | | | | DOG2 | | | |
| 1-27a-e | 1-1 | — | — | — | 31 | Acidobacteria | 21 | Zeocin | 37 |
| | | | | | | bacterium Sugar | | | |
| | | | | | | Phosphatase | | | |
| | | | | | | (PYX10474) | | | |
| 1-28a-c | 1-1 | — | — | — | 34 | Puerhibacterium | 24 | Zeocin | 37 |
| | | | | | | puerhi Sugar | | | |
| | | | | | | Phosphatase | | | |
| | | | | | | (WP_159795540) | | | |

### Example 2—Shake Flask Fermentation Assay

Strains 1-2a-c, 1-3a-c, 1-4a-c, 1-5a-c, 1-6a-c, 1-7a-c, 1-8a-c, 1-9a-c, 1-10a-c, 1-11a-c, and 1-12a-c (outlined in Table 2 above), were run in shake flasks to assess xylitol, erythritol, ribitol, glycerol, arabitol, and ethanol production and glucose consumption.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

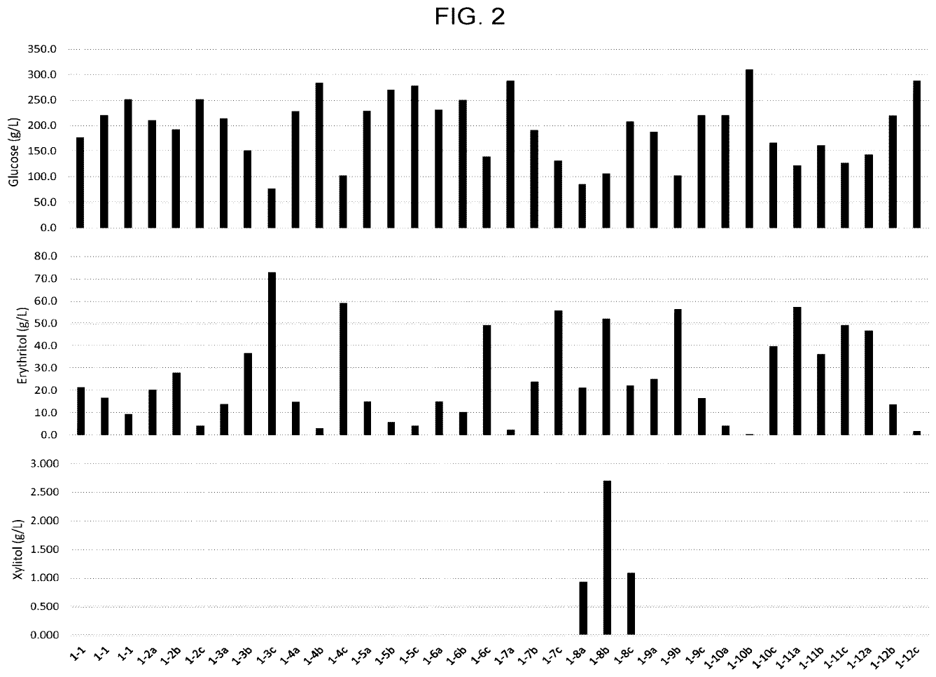

A 250 ml non-baffled flask containing production medium (Table C) was inoculated with the seed culture to form the production culture with a starting OD600 of about 0.4 (approximately 0.8 mL of the seed culture). The production culture was incubated at 35° C. and 250 rpm. Samples were taken from the production culture after 96 hours of incubation. Samples were analyzed for glucose, ribitol, xylitol, erythritol, glycerol, arabitol, and ethanol by high performance liquid chromatography with refractive index detector. Fermentation results are reported in Table 5 and FIG. 2. The expression of both the Pichia stipitis cofactor switched XDH (SEQ ID NO:1) and the Saccharomyces cerevisiae DOG1 sugar phosphatase (SEQ ID NO:8) in Moniliella pollinis resulted in production of xylitol.

TABLE 4
Production Medium

| | Concentration |
|---|---|
| Component | (units) |
| Glucose | 300 (g/L) |
| KH2PO4 | 1.27 (g/L) |
| (NH4)2HPO4 | 0.13 (g/L) |
| (NH4)2SO4 | 1.80 (g/L) |
| Urea | 2.85 (g/L) |
| Citric Acid | 200.0 (mg/L) |
| MgSO4-7H2O | 690.2 (mg/L) |
| FeSO4-7H2O | 32.35 (mg/L) |
| ZnSO4-7H2O | 4.40 (mg/L) |
| MnSO4-H2O | 0.53 (mg/L) |
| CuSO4-5H2O | 0.45 (mg/L) |
| CaCl2-2H2O | 91.60 (mg/L) |
| Na2SO4 | 154.50 (mg/L) |
| Thiamine-HCl | 10 (mg/L) |
| Choline-Cl | 100 (mg/L) |
| Antifoam CF-32 | 1.00 (g/L) |

TABLE 5
96 hour Shake Flask Results

| | Fermentation Broth Analyte (g/L) | | | | | | |
|---|---|---|---|---|---|---|---|
| Strain | Glucose | Ribitol | Erythritol | Glycerol | Ethanol | Xylitol | Arabitol |
| 1-1 | 177.3 | 0.3 | 21.3 | 2.6 | 24.1 | 0.000 | 0.00 |
| 1-1 | 221.2 | 0.6 | 16.7 | 8.4 | 9.5 | 0.000 | 0.00 |
| 1-1 | 251.5 | 0.4 | 9.4 | 6.8 | 3.0 | 0.000 | 0.00 |
| 1-2a | 210.2 | 0.8 | 20.3 | 8.9 | 10.2 | 0.000 | 0.00 |
| 1-2b | 192.1 | 0.6 | 28.0 | 8.4 | 14.6 | 0.000 | 0.00 |
| 1-2c | 251.3 | 0.2 | 4.2 | 4.0 | 1.1 | 0.000 | 0.00 |
| 1-3a | 214.3 | 1.6 | 13.8 | 9.3 | 12.1 | 0.000 | 0.00 |
| 1-3b | 151.4 | 0.6 | 36.8 | 9.6 | 24.3 | 0.000 | 0.00 |
| 1-3c | 77.0 | 1.6 | 72.9 | 13.4 | 29.9 | 0.000 | 0.00 |
| 1-4a | 228.4 | 0.5 | 14.9 | 7.3 | 8.3 | 0.000 | 0.00 |
| 1-4b | 283.5 | 0.3 | 3.1 | 2.8 | 1.2 | 0.000 | 0.00 |
| 1-4c | 102.6 | 1.3 | 59.1 | 12.3 | 30.9 | 0.000 | 0.00 |
| 1-5a | 229.0 | 0.5 | 14.9 | 6.7 | 8.3 | 0.000 | 0.00 |
| 1-5b | 270.5 | 0.3 | 5.9 | 5.0 | 1.9 | 0.000 | 0.00 |
| 1-5c | 278.4 | 0.6 | 4.2 | 3.9 | 1.0 | 0.000 | 0.00 |
| 1-6a | 231.3 | 0.4 | 14.9 | 6.0 | 8.4 | 0.000 | 0.00 |
| 1-6b | 250.1 | 0.4 | 10.2 | 6.4 | 4.0 | 0.000 | 0.00 |
| 1-6c | 139.7 | 0.9 | 49.2 | 10.0 | 22.3 | 0.000 | 0.00 |
| 1-7a | 287.9 | 0.1 | 2.3 | 1.2 | 2.0 | 0.000 | 0.00 |
| 1-7b | 191.2 | 0.9 | 23.9 | 7.1 | 16.9 | 0.000 | 0.00 |
| 1-7c | 131.5 | 0.9 | 55.7 | 7.6 | 23.5 | 0.000 | 0.00 |
| 1-8a | 85.0 | 3.4 | 21.2 | 6.2 | 14.9 | 0.931 | 0.00 |
| 1-8b | 105.8 | 7.6 | 52.0 | 10.3 | 27.6 | 2.699 | 0.00 |
| 1-8c | 208.3 | 2.7 | 22.2 | 6.2 | 11.0 | 1.092 | 0.00 |
| 1-9a | 187.7 | 0.9 | 25.0 | 8.5 | 17.9 | 0.000 | 0.00 |
| 1-9b | 102.4 | 1.5 | 56.3 | 19.5 | 26.1 | 0.000 | 0.00 |
| 1-9c | 221.1 | 0.8 | 16.6 | 5.7 | 10.8 | 0.000 | 0.00 |
| 1-10a | 221.0 | 0.2 | 4.1 | 3.0 | 1.9 | 0.000 | 0.00 |
| 1-10b | 309.7 | 0.8 | 0.3 | 0.7 | 0.5 | 0.000 | 0.00 |
| 1-10c | 166.4 | 0.0 | 39.6 | 8.4 | 18.7 | 0.000 | 0.00 |
| 1-11a | 121.9 | 1.2 | 57.4 | 6.2 | 25.2 | 0.000 | 0.00 |
| 1-11b | 161.8 | 1.0 | 36.2 | 7.8 | 16.8 | 0.000 | 0.00 |
| 1-11c | 126.9 | 1.1 | 49.1 | 13.4 | 26.1 | 0.000 | 0.00 |
| 1-12a | 143.4 | 1.0 | 46.8 | 9.3 | 22.7 | 0.000 | 0.00 |
| 1-12b | 219.8 | 0.7 | 13.7 | 8.9 | 9.4 | 0.000 | 0.00 |
| 1-12c | 288.3 | 0.1 | 1.8 | 0.9 | 4.3 | 0.000 | 0.11 |

### Example 3—Shake Flask Fermentation Assay

Strains 1-8a-c, 1-13, 1-14a and 1-14c (outlined in Table 2 above), were run in duplicate in shake flasks to assess xylitol, erythritol, ribitol, glycerol, and ethanol production and glucose consumption.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

Either a 250 ml non-baffled flask or a 250 ml baffled flask containing production medium (Table B) was inoculated with 0.8 mL of the seed culture to form the production culture. The production culture was incubated at 35° C. and 250 rpm. Samples were taken from the production culture after 48, 72, and 96 hours of incubation. Samples were analyzed for glucose, arabitol, ribitol, xylitol, erythritol, glycerol, and ethanol by high performance liquid chromatography with refractive index detector. Fermentation results are reported in Table 6 and FIG. 2. As demonstrated in the results, expression of the Saccharomyces cerevisiae DOG1 sugar phosphatase (SEQ ID NO:9) in combination with either the Pichia stipitis cofactor switched XDH (SEQ ID NO:1), the Rhodobacteraceae bacterium SDR family oxidoreductase (SEQ ID NO:13), or the Bemisia argentofolii (Silverleaf Whitefly) ketose reductase (sorbitol dehydrogenase) (SEQ ID NO:14) in Moniliella pollinis produced xylitol after as little as 48 hours of fermentation.

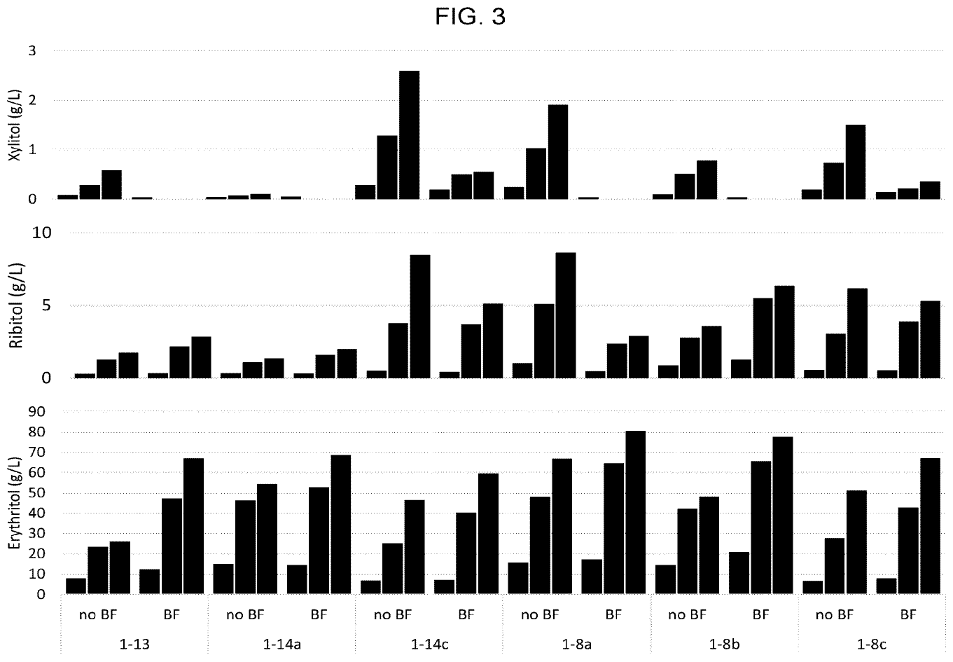

While all strains tested produced xylitol, the level of production can be changed by altering the aeration of the fermentation. For example, as shown in FIG. 3, with high aeration (i.e., fermentation done in a baffled flask) strain 1-14c produced about 0.5 g/L xylitol after 96 hours, however fermentation at lower aeration (i.e., in a non-baffled flask) resulted in xylitol production over 2.5 g/L.

While PCR verification indicated that the transformed polynucleotide sequence was present in the indicated strains, further analysis indicated that in some strains, the sequences was not correctly targeted to the ER1 loci or multiple copies were integrated. Further analysis indicated that strains 1-14b and 1-13 include the transformed polynucleotide sequence, but it is not targeted to the ER1 locus. Strain 1-14b was not run in this shake flask experiment due to resource limitations. Further analysis also indicated that strain 1-14c included 8 copies of the polynucleotide encoding SEQ ID NO:14 and 10 copies of the polynucleotide encoding SEQ ID NO:8, but none of the sequences were correctly targeted to the ER1 locus.

TABLE 6
Shake Flask Results

| | Time | Baffled | Fermentation Broth Analyte (g/L) | | | | | |
|---|---|---|---|---|---|---|---|---|
| Strain | Point | Flask | Glucose | Ribitol | Xylitol | Erythritol | Glycerol | Ethanol |
| 1-8a | 48 | no | 217.948 | 1.0374 | 0.2464 | 15.666 | 10.4687 | 11.1309 |
| 1-8a | 48 | no | 218.631 | 1.0375 | 0.2338 | 15.3798 | 10.3807 | 11.0564 |
| 1-8b | 48 | no | 219.0666 | 0.8671 | 0.0996 | 14.5724 | 10.0748 | 13.259 |
| 1-8b | 48 | no | 213.3015 | 0.8602 | 0.1576 | 15.2142 | 10.2649 | 13.6549 |
| 1-8c | 48 | no | 256.7603 | 0.5482 | 0.1941 | 6.6763 | 3.9799 | 5.2907 |
| 1-8c | 48 | no | 256.422 | 0.5651 | 0.1815 | 6.6742 | 3.8904 | 5.3232 |
| 1-13 | 48 | no | 246.6843 | 0.3063 | 0.0831 | 7.9747 | 5.5501 | 7.7021 |
| 1-13 | 48 | no | 251.1016 | 0.3189 | 0.1035 | 8.5806 | 5.9284 | 8.0667 |
| 1-14a | 48 | no | 227.6182 | 0.3365 | 0.047 | 14.9907 | 13.0872 | 1.4197 |
| 1-14a | 48 | no | 230.0448 | 0.2339 | 0.0757 | 10.1985 | 5.5809 | 12.2101 |
| 1-14c | 48 | no | 262.5613 | 0.5171 | 0.2866 | 6.8507 | 4.5567 | 5.6778 |
| 1-14c | 48 | no | 257.487 | 0.524 | 0.2912 | 6.6819 | 4.047 | 5.7798 |
| 1-8a | 48 | yes | 225.6687 | 0.4823 | 0.0394 | 17.2254 | 18.4131 | 1.2625 |
| 1-8a | 48 | yes | 221.4495 | 0.501 | 0.0209 | 17.7996 | 18.7213 | 1.2621 |
| 1-8b | 48 | yes | 201.8136 | 1.2746 | 0.0416 | 20.798 | 24.7817 | 2.0304 |
| 1-8b | 48 | yes | 193.9384 | 1.4128 | 0.0485 | 22.8222 | 27.5048 | 1.9007 |
| 1-8c | 48 | yes | 262.1726 | 0.5381 | 0.1465 | 7.9166 | 6.2902 | 1.1757 |
| 1-8c | 48 | yes | 264.5841 | 0.4884 | 0.139 | 7.2657 | 5.4647 | 1.1227 |
| 1-13 | 48 | yes | 241.0328 | 0.3489 | 0.039 | 12.3663 | 11.3904 | 0.53 |
| 1-13 | 48 | yes | 237.0301 | 0.3694 | 0.0285 | 13.3439 | 12.7668 | 0.5016 |
| 1-14a | 48 | yes | 229.0956 | 0.3295 | 0.0531 | 14.5186 | 12.3315 | 2.179 |
| 1-14a | 48 | yes | 219.8367 | 0.3411 | 0.0338 | 16.4859 | 14.9275 | 1.7324 |
| 1-14c | 48 | yes | 260.7984 | 0.4379 | 0.2001 | 7.216 | 5.4117 | 1.0756 |
| 1-14c | 48 | yes | 260.4357 | 0.4595 | 0.2134 | 7.4861 | 5.8055 | 1.1902 |
| 1-8a | 72 | no | 118.9917 | 5.1176 | 1.0347 | 48.021 | 14.6104 | 22.6909 |
| 1-8a | 72 | no | 121.258 | 5.0107 | 1.0405 | 47.5303 | 14.4495 | 22.7773 |
| 1-8b | 72 | no | 100.3422 | 2.7894 | 0.5144 | 42.2335 | 14.1556 | 34.3714 |
| 1-8b | 72 | no | 95.5028 | 2.8572 | 0.4867 | 43.7333 | 14.2579 | 34.2852 |
| 1-8c | 72 | no | 187.1468 | 3.0444 | 0.7336 | 27.5958 | 5.8653 | 14.635 |
| 1-8c | 72 | no | 187.8452 | 3.0079 | 0.7989 | 27.3219 | 5.7335 | 14.8461 |
| 1-13 | 72 | no | 146.0341 | 1.2619 | 0.2846 | 23.3061 | 7.5611 | 33.3918 |
| 1-13 | 72 | no | 142.3538 | 1.2522 | 0.372 | 24.8359 | 7.9639 | 33.7677 |
| 1-14a | 72 | no | 93.014 | 1.0903 | 0.0729 | 46.3431 | 24.2313 | 19.8416 |
| 1-14a | 72 | no | 116.7176 | 0.6386 | 0.1422 | 24.6448 | 7.1184 | 45.0416 |
| 1-14c | 72 | no | 192.5862 | 3.7735 | 1.2879 | 25.1962 | 6.1937 | 14.9647 |
| 1-14c | 72 | no | 188.6326 | 3.8733 | 1.2983 | 26.1231 | 5.7399 | 14.0219 |
| 1-8a | 72 | yes | 85.7327 | 2.3671 | 0 | 64.5607 | 50.4989 | 0.3506 |
| 1-8a | 72 | yes | 84.8898 | 2.3079 | 0 | 65.2192 | 50.0771 | 0.3258 |
| 1-8b | 72 | yes | 51.5689 | 5.4855 | 0 | 65.6227 | 59.0813 | 2.8692 |
| 1-8b | 72 | yes | 43.6927 | 5.1612 | 0 | 68.9601 | 65.7296 | 0.2363 |
| 1-8c | 72 | yes | 145.4187 | 3.8764 | 0.2167 | 42.7845 | 26.0502 | 3.2431 |
| 1-8c | 72 | yes | 151.2918 | 3.4321 | 0.2388 | 40.3376 | 24.5156 | 2.5922 |
| 1-13 | 72 | yes | 116.7837 | 2.1609 | 0 | 47.3081 | 36.4069 | 1.4031 |
| 1-13 | 72 | yes | 109.7339 | 2.1555 | 0 | 48.744 | 39.4191 | 1.5179 |
| 1-14a | 72 | yes | 98.9757 | 1.5879 | 0 | 52.8139 | 35.1409 | 4.7515 |
| 1-14a | 72 | yes | 89.9746 | 1.5549 | 0 | 54.1916 | 37.243 | 6.0006 |
| 1-14c | 72 | yes | 147.7416 | 3.6853 | 0.4986 | 40.2659 | 23.9122 | 2.6688 |
| 1-14c | 72 | yes | 142.0211 | 3.7815 | 0.473 | 40.3568 | 23.2301 | 5.5432 |
| 1-8a | 96 | no | 42.8644 | 8.6314 | 1.9033 | 66.7209 | 16.8236 | 35.6326 |
| 1-8a | 96 | no | 43.306 | 8.7595 | 1.8279 | 67.2738 | 16.6128 | 36.069 |
| 1-8b | 96 | no | 42.576 | 3.5921 | 0.7858 | 48.1976 | 15.0165 | 47.2719 |
| 1-8b | 96 | no | 39.3759 | 3.6963 | 0.8457 | 50.5586 | 15.2643 | 47.3769 |
| 1-8c | 96 | no | 121.1279 | 6.1571 | 1.5101 | 51.1161 | 7.7732 | 24.5364 |
| 1-8c | 96 | no | 119.7203 | 5.8701 | 1.5268 | 49.7403 | 7.4936 | 24.1627 |
| 1-13 | 96 | no | 86.8157 | 1.7625 | 0.5887 | 26.0683 | 8.171 | 49.5427 |
| 1-13 | 96 | no | 81.3414 | 1.8161 | 0.5429 | 28.1024 | 8.695 | 51.9138 |
| 1-14a | 96 | no | 67.2068 | 1.3508 | 0.1064 | 54.414 | 23.4538 | 18.5858 |
| 1-14a | 96 | no | 68.7416 | 0.8149 | 0.2827 | 25.286 | 7.6839 | 57.539 |
| 1-14c | 96 | no | 126.5677 | 8.4837 | 2.5892 | 46.5038 | 7.8006 | 20.4329 |
| 1-14c | 96 | no | 123.4764 | 8.2189 | 2.5542 | 47.1131 | 7.3021 | 20.5738 |
| 1-8a | 96 | yes | 53.3442 | 2.8951 | 0 | 80.6748 | 54.7071 | 0.2221 |
| 1-8a | 96 | yes | 51.6395 | 2.8795 | 0 | 82.5517 | 54.7041 | 0.1745 |
| 1-8b | 96 | yes | 29.5099 | 6.3734 | 0 | 77.4994 | 64.3626 | 1.7047 |
| 1-8b | 96 | yes | 24.9716 | 5.5745 | 0 | 76.997 | 69.4787 | 0.1281 |
| 1-8c | 96 | yes | 91.2815 | 5.298 | 0.3619 | 67.0619 | 34.1619 | 1.2364 |
| 1-8c | 96 | yes | 92.6441 | 5.1885 | 0.2913 | 67.3122 | 34.9422 | 0.474 |
| 1-13 | 96 | yes | 74.1743 | 2.8581 | 0 | 66.9987 | 45.3131 | 0.21 |
| 1-13 | 96 | yes | 69.5317 | 2.8072 | 0 | 67.201 | 47.9707 | 0.2132 |
| 1-14a | 96 | yes | 62.1225 | 1.9868 | 0 | 68.7981 | 40.2958 | 2.7787 |
| 1-14a | 96 | yes | 58.7857 | 1.9428 | 0 | 69.8722 | 42.3314 | 4.2523 |
| 1-14c | 96 | yes | 98.3003 | 5.1312 | 0.5472 | 59.6042 | 27.5231 | 4.4645 |
| 1-14c | 96 | yes | 95.5057 | 5.3736 | 0.5769 | 64.4065 | 32.0783 | 1.0075 |

### Example 4—Shake Flask Fermentation Assay

Strains 1-14c, 1-15a-d, 1-16a-d, 1-17a-e, 1-18a-e, and 1-19a-e (outlined in Tables A and B above), were run in shake flasks to assess xylitol, erythritol, ribitol, glycerol, and ethanol production and glucose consumption.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

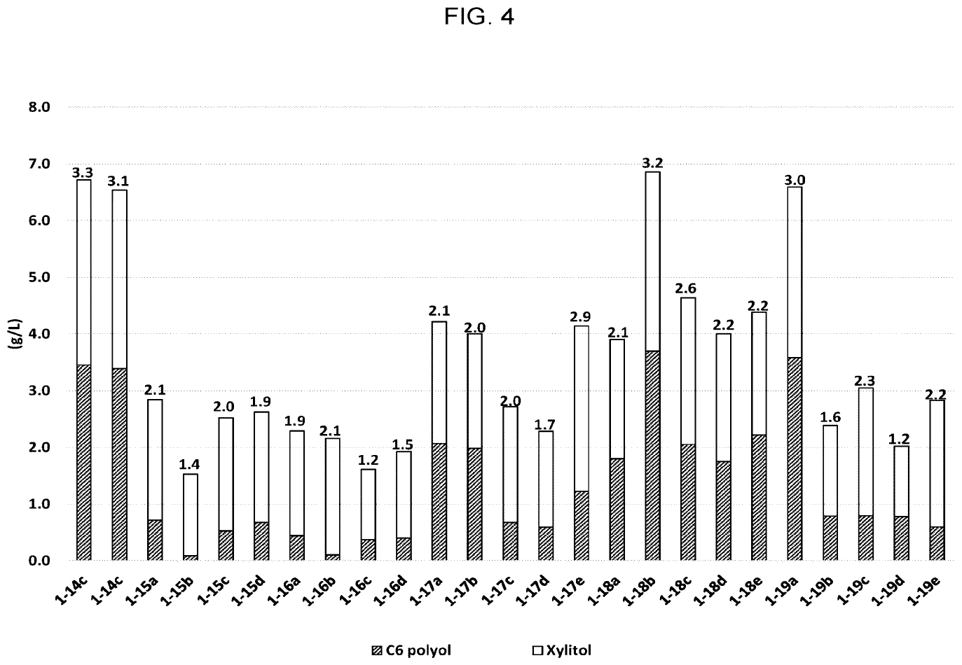

A 250 ml non-baffled flask containing production medium (Table C) was inoculated with 0.8 mL of the seed culture to form the production culture. The production culture was incubated at 35° C. and 250 rpm. Samples were taken from the production culture after 96 hours of incubation. Samples were analyzed for glucose, arabitol, ribitol, xylitol, erythritol, glycerol, and ethanol by high performance liquid chromatography with refractive index detector. Fermentation results are reported in Table 7 and FIG. 4.

As outlined in Table 2, strain 1-14c was transformed with one of the recited XDH homolog candidates. Shake flask characterization of these strains demonstrated that none of the tested XDH homolog candidates increased xylitol production above the production level of the 1-14c parent strain under the same conditions. In fact, some strains had decreased xylitol production relative to the parent strain.

While PCR verification indicated that the transformed polynucleotide sequence was present in the indicated strains, further analysis indicated that in some strains, the sequences was not correctly targeted to the ER1 locus. Further analysis indicated that strains 1-15a-d, 1-16a-d, 1-17a-e, 1-18a-e, and 1-19a-e include the transformed polynucleotide sequence, but it is not targeted to the ER1 locus.

TABLE 7
96 hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-14c | 99.4 | 9.2 | 3.3 | 50.2 | 8.0 | 31.0 |
| 1-14c | 97.0227 | 9.5367 | 3.1468 | 51.6515 | 7.91 | 31.5191 |
| 1-15a | 124.5137 | 6.615 | 2.1213 | 53.0056 | 6.7519 | 26.1911 |
| 1-15b | 176.5 | 3.8 | 1.4 | 41.1 | 6.8 | 14.3 |
| 1-15c | 158.5056 | 8.3277 | 1.9897 | 46.3165 | 6.3899 | 16.2085 |
| 1-15d | 169.4034 | 7.4558 | 1.9438 | 40.7081 | 6.8775 | 16.2906 |
| 1-16a | 183.2909 | 5.7001 | 1.8513 | 37.6993 | 6.532 | 13.9718 |
| 1-16b | 195.2726 | 5.9215 | 2.0525 | 34.291 | 7.8959 | 10.4399 |
| 1-16c | 186.0142 | 3.0993 | 1.2395 | 38.5607 | 6.4959 | 13.3875 |
| 1-16d | 220.4559 | 4.0252 | 1.5282 | 26.3763 | 5.2339 | 8.364 |
| 1-17a | 93.7651 | 7.9426 | 2.1475 | 61.8649 | 10.1967 | 30.4088 |
| 1-17b | 109.4079 | 7.1854 | 2.0227 | 56.4907 | 8.9673 | 29.4155 |
| 1-17c | 151.9165 | 7.2493 | 2.0357 | 47.4785 | 7.341 | 19.2757 |
| 1-17d | 155.3155 | 5.1622 | 1.6969 | 46.4501 | 7.0876 | 19.89 |
| 1-17e | 155.6752 | 9.3248 | 2.9191 | 45.036 | 7.2426 | 19.2712 |
| 1-18a | 72.4171 | 5.9224 | 2.1062 | 65.4266 | 9.9271 | 35.9972 |
| 1-18b | 91.4329 | 9.5635 | 3.1593 | 55.0515 | 8.7798 | 32.4985 |
| 1-18c | 127.842 | 7.0101 | 2.5856 | 47.4543 | 7.4967 | 26.2408 |
| 1-18d | 91.8716 | 6.3103 | 2.2494 | 57.7429 | 7.5496 | 36.6871 |
| 1-18e | 80.0458 | 6.3959 | 2.164 | 60.4785 | 9.5805 | 37.3784 |
| 1-19a | 89.4024 | 8.9136 | 3.0074 | 55.2153 | 8.9666 | 34.287 |
| 1-19b | 114.2333 | 5.5967 | 1.5941 | 60.2268 | 8.8782 | 27.7048 |
| 1-19c | 157.0952 | 7.9909 | 2.2512 | 46.2085 | 7.5793 | 17.9777 |
| 1-19d | 85.8555 | 3.6704 | 1.2423 | 67.7445 | 9.3857 | 35.5171 |
| 1-19e | 166.3217 | 4.1078 | 2.2277 | 40.8836 | 5.8864 | 21.8067 |

### Example 5—Shake Flask Fermentation Assay

Strains 1-14c, 1-20a-e, 1-21a-e, 1-23a-e, and 1-24a-e (outlined in Tables A and B above), were run in shake flasks to assess xylitol, erythritol, ribitol, glycerol, and ethanol production and glucose consumption.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

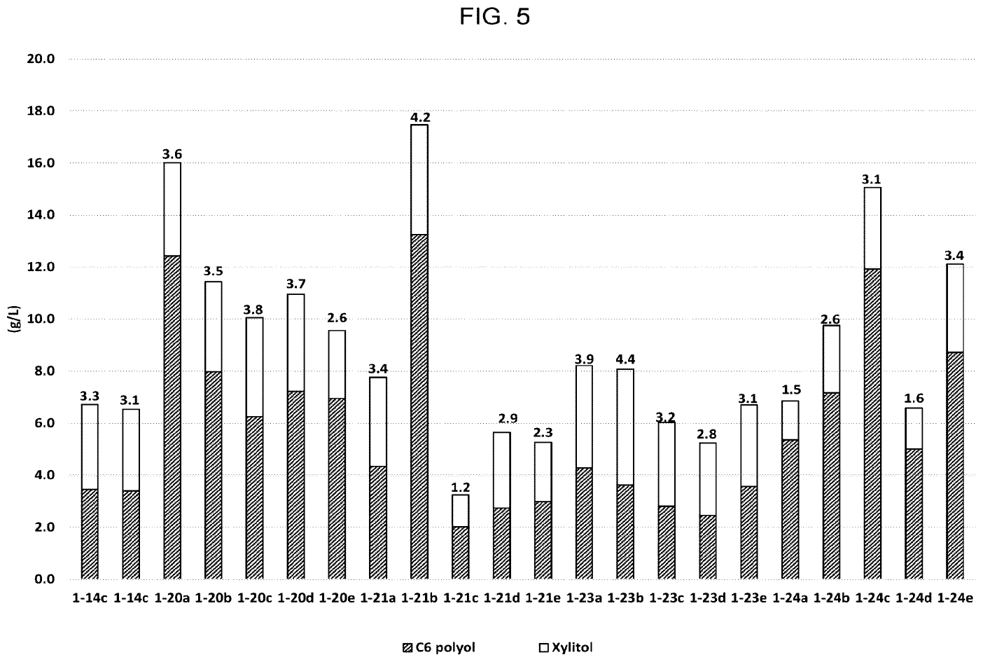

A 250 ml non-baffled flask containing production medium (Table C) was inoculated with 0.8 mL of the seed culture to form the production culture. The production culture was incubated at 35° C. and 250 rpm. Samples were taken from the production culture after 96 hours of incubation. Samples were analyzed for glucose, arabitol, ribitol, xylitol, erythritol, glycerol, and ethanol by high performance liquid chromatography with refractive index detector. Fermentation results are reported in Table 8 and FIG. 5.

As outlined in Table 2, strain 1-14c was transformed with one of the recited XKS/sugar phosphatase homolog candidates. Strains expressing the S. cerevisiae DOG2 sugar phosphatase, A. bacterium phosphatase homolog, or the P. puerhi HAD-IA family hydrolase homolog had increased final titers of xylitol titers. Results for the Mesorhizobium sp. HAD family hydrolase homolog candidate were similar to those reported for the E. coli hexitol phosphatase gene (data not shown).

While PCR verification indicated that the transformed polynucleotide sequence was present in the indicated strains, further analysis indicated that in some strains, the sequences was not correctly targeted to the ER1 locus. Further analysis indicated that strains 1-20a-e, 1-21a-e, 1-23a-e, and 1-24a-e include the transformed polynucleotide sequence, but it is not targeted to the ER1 locus.

TABLE 8
96 hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-14c | 99.4 | 9.2 | 3.3 | 50.2 | 8.0 | 31.0 |
| 1-14c | 97.0227 | 9.5367 | 3.1468 | 51.6515 | 7.91 | 31.5191 |
| 1-20a | 109.8471 | 5.3636 | 3.5797 | 40.621 | 7.616 | 33.8712 |
| 1-20b | 88.5895 | 8.2061 | 3.4587 | 50.5238 | 8.2101 | 35.4361 |
| 1-20c | 194.6963 | 5.1945 | 3.8179 | 21.9057 | 3.667 | 14.9874 |
| 1-20d | 158.3447 | 6.5005 | 3.7387 | 33.7401 | 4.7477 | 22.013 |
| 1-20e | 121.7546 | 5.3639 | 2.6149 | 49.5375 | 6.4906 | 29.9388 |
| 1-21a | 81.7409 | 11.0912 | 3.4072 | 55.3219 | 9.1535 | 34.8535 |
| 1-21b | 79.2342 | 11.539 | 4.2206 | 28.6582 | 9.8801 | 47.4776 |
| 1-21c | 95.6748 | 3.4423 | 1.23 | 57.8996 | 9.8309 | 37.3651 |
| 1-21d | 134.4472 | 7.3051 | 2.9165 | 40.3628 | 7.1966 | 28.6082 |
| 1-21e | 71.2417 | 8.4875 | 2.281 | 70.1432 | 9.6048 | 37.6368 |
| 1-23a | 99.7368 | 11.7962 | 3.9377 | 55.9439 | 8.4156 | 30.7757 |
| 1-23b | 98.576 | 6.8823 | 4.4492 | 42.9345 | 8.5033 | 40.8846 |
| 1-23c | 105.586 | 9.9521 | 3.2203 | 52.3807 | 8.7781 | 32.1227 |
| 1-23d | 86.3296 | 3.0776 | 2.7847 | 52.2668 | 11.9752 | 39.491 |
| 1-23e | 88.5566 | 8.441 | 3.1327 | 56.6528 | 7.5047 | 36.5921 |
| 1-24a | 98.2495 | 2.8919 | 1.4898 | 59.1482 | 7.474 | 32.5949 |
| 1-24b | 115.6181 | 8.1052 | 2.586 | 47.4175 | 7.1073 | 29.597 |
| 1-24c | 120.0083 | 9.3171 | 3.1328 | 52.7683 | 5.2785 | 25.0334 |
| 1-24d | 92.7436 | 3.1304 | 1.5755 | 63.0015 | 7.9723 | 34.7408 |
| 1-24e | 115.923 | 9.2322 | 3.3841 | 52.8348 | 7.1139 | 25.719 |

### Example 6—Shake Flask Fermentation Assay

Strains 1-1, 1-20b, 1-24e, 1-25a-d, 1-26a-e, 1-27a-e, and 1-28a-c (outlined in Tables A and B above), were run in shake flasks in duplicate to assess xylitol, erythritol, ribitol, glycerol, and ethanol production and glucose consumption.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

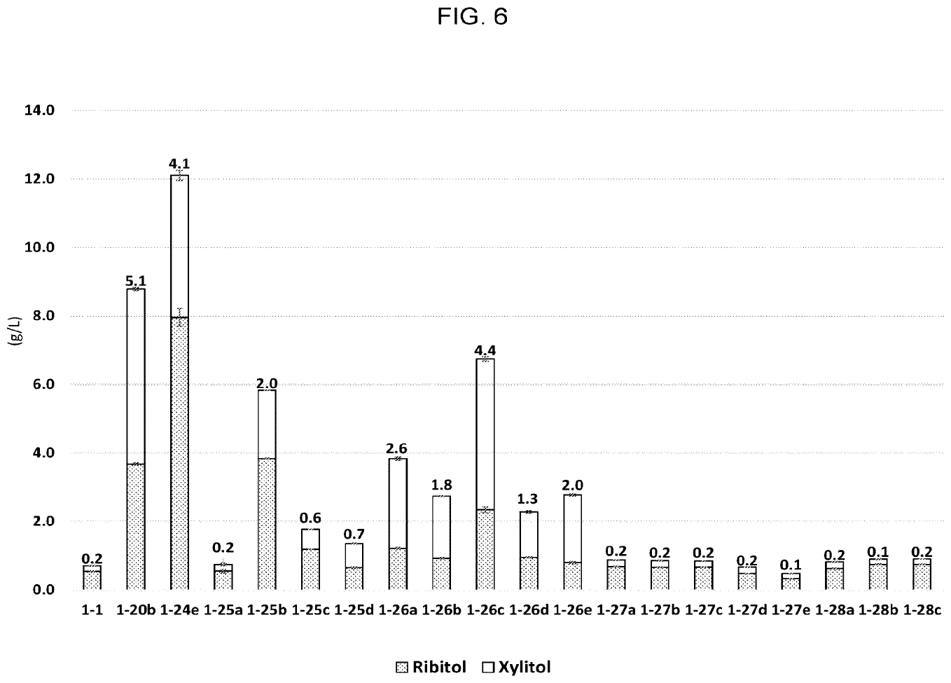

A 250 ml non-baffled flask containing production medium (Table C) was inoculated with 0.8 mL of the seed culture to form the production culture. The production culture was incubated at 35° C. and 250 rpm. Samples were taken from the production culture after 96 hours of incubation. Samples were analyzed for glucose, arabitol, ribitol, xylitol, erythritol, glycerol, and ethanol by high performance liquid chromatography with refractive index detector. Fermentation results are reported in Table 9 and FIG. 6.

The strains tested in this example include the phosphatase candidates that produced increased xylitol titers in Example 5 transformed into the wild-type background to observe the metabolite production. Strains expressing either the S. cerevisiae DOG1 or the S. cerevisiae DOG2 sugar phosphatase enzymes produced more xylitol than the wild-type parent background.

While PCR verification indicated that the transformed polynucleotide sequence was present in the indicated strains, further analysis indicated that in some strains, the sequences was not correctly targeted to the ER1 locus. Further analysis indicated that strains 1-20b, 1-24e, 1-25a-d, 1-26a-c, 1-27a-c, and 1-28a-b include the transformed polynucleotide sequence, but it is not targeted to the ER1 locus.

TABLE 9
Average 96 hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-1 | 65.84 | 0.54 | 0.17 | 53.37 | 16.13 | 40.53 |
| 1-20b | 72.25 | 3.68 | 5.10 | 28.32 | 8.51 | 51.17 |
| 1-24e | 93.81 | 7.96 | 4.15 | 41.51 | 7.66 | 35.83 |
| 1-25a | 110.33 | 0.55 | 0.20 | 34.05 | 16.34 | 34.64 |
| 1-25b | 57.89 | 3.83 | 2.00 | 46.75 | 13.08 | 48.28 |
| 1-25c | 30.80 | 1.18 | 0.58 | 52.12 | 24.52 | 50.73 |
| 1-25d | 88.74 | 0.65 | 0.70 | 40.08 | 20.89 | 38.58 |
| 1-26a | 103.26 | 1.21 | 2.62 | 31.62 | 10.16 | 39.15 |
| 1-26b | 104.19 | 0.92 | 1.82 | 34.26 | 11.94 | 38.68 |
| 1-26c | 100.23 | 2.34 | 4.40 | 25.45 | 6.34 | 43.85 |
| 1-26d | 47.48 | 0.95 | 1.33 | 64.81 | 10.16 | 46.51 |
| 1-26e | 83.76 | 0.80 | 1.97 | 34.24 | 11.14 | 45.99 |
| 1-27a | 65.19 | 0.68 | 0.19 | 58.14 | 16.17 | 40.48 |
| 1-27b | 62.19 | 0.66 | 0.19 | 55.95 | 16.20 | 43.71 |
| 1-27c | 68.36 | 0.67 | 0.18 | 56.33 | 15.43 | 41.59 |
| 1-27d | 86.03 | 0.48 | 0.19 | 45.27 | 15.81 | 39.76 |
| 1-27e | 98.16 | 0.33 | 0.15 | 37.02 | 13.95 | 38.39 |
| 1-28a | 94.61 | 0.63 | 0.19 | 48.79 | 14.47 | 35.63 |
| 1-28b | 68.15 | 0.75 | 0.15 | 56.16 | 13.31 | 41.42 |
| 1-28c | 78.41 | 0.75 | 0.15 | 54.84 | 21.73 | 38.09 |

1. A genetically engineered yeast cell capable of producing xylitol, the engineered yeast cell comprising:
an exogenous polynucleotide sequence encoding a xylulokinase (XKS) enzyme comprising a sequence at least 80% identical to at least one of SEQ ID NOs:8 and 20.

2. The yeast cell of claim 1, wherein the yeast cell is an osmotolerant yeast cell.

3. The yeast cell of claim 1, wherein the yeast cell is a cell of the subphylum Ustilaginomycotina.

4. The yeast cell of claim 1, wherein the yeast cell is selected from the group consisting of Trichosporonoides megachiliensis, Trychosporonoides oedocephalis, Trychosporonoides nigrescens, Pseudozyma tsukubaensis, Trigonopsis variabilis, Moniliella, Ustilaginomycetes, Trichosporon, Yarrowia lipolytica, Penicillium, Torula, Pichia, Candida, Candida magnoliae, and Aureobasidium.

5. The yeast cell of claim 1, wherein the yeast cell is a yeast cell of the Moniliella genus.

6. (canceled)

7. The yeast cell of claim 5, wherein the cell is a Moniliella pollinis cell.

8. The yeast cell of claim 1, wherein the yeast cell is capable of producing xylitol at a titer of at least 1.0 g/L when used in a fermentation process in the presence of dextrose for 96 hours.

9. The yeast cell of claim 1, wherein the exogenous polynucleotide sequence is integrated into the genome of the yeast cell at a locus selected from the ER1 locus, the ER3 locus, the PDC1 locus, the pyrF locus, the TRP3 locus, the gpdIIA locus, and the gpdIIB locus.

10. The yeast cell of claim 1, wherein the exogenous polynucleotide sequence is operably linked to a heterologous or artificial promoter selected from the group consisting of pyruvate kinase 1 promoter (PYK1p: SEQ ID NO:61), 6-phosphogluconate dehydrogenase promoter (6PGDp; SEQ ID NO:52), glyceraldehyde-3-phosphate dehydrogenase promoter (TDH3p; SEQ ID NO:54), translational elongation factor 1 promoter (TEFp; SEQ ID NO:55), modified TEFp (SEQ ID NO:53), phosphoglucomutase 1 promoter (PGM1p: SEQ ID NO:56), 3-phosphoglycerate kinase promoter (PGK1p; SEQ ID NO:57), enolase promoter (ENO1p; SEQ ID NO:58), asparagine synthetase promoter (ASNSp; SEQ ID NO:59), 50S ribosomal protein L1 promoter (RPLAp; SEQ ID NO:60), and RPL16B (SEQ ID NO:62).

11. (canceled)

12. (canceled)

13. The yeast cell of claim 1, wherein the yeast cell additionally comprises
an exogenous polynucleotide sequence encoding a xylitol dehydrogenase (XDH) enzyme comprising a sequence at least 80% identical to at least one of SEQ ID NOs:1, 13, and 14.

14. (canceled)

15. (canceled)

16. The yeast cell of claim 1, wherein the sugar phosphatase enzyme has a sequence at least 85% identical to at least one of SEQ ID NOs:8 and 20.

17. The yeast cell of claim 1, wherein the sugar phosphatase enzyme has a sequence at least 90% identical to at least one of SEQ ID NOs:8 and 20.

18. The yeast cell of claim 13, wherein the XHD enzyme has a sequence at least 85% identical to at least one of SEQ ID NOs:1, 13, and 14.

19. The yeast cell of claim 13, wherein the XDH enzyme has a sequence at least 90% identical to at least one of SEQ ID NOs:1, 13, and 14.

20. A method for producing xylitol, the method comprising:
contacting a substrate comprising dextrose with the engineered yeast cell of claim 1, wherein fermentation of the substrate by the engineered yeast produces xylitol.

21. (canceled)

22. (canceled)

23. The method of claim 20, wherein the fermentation temperature is at or between 25° C. to 45° C.

24. The method of claim 20, wherein xylitol production is at least 1.0 g/L when the fermentation is run at 35° C. for 96 hours.

25. (canceled)

26. (canceled)

27. The method claim 20, wherein the volumetric oxygen uptake rate (OUR) is between 0.5 to 40 mmol O₂/(L·h).

28. (canceled)

29. (canceled)

30. (canceled)
